(19) United States
(12) Patent Application Publication
(10) Pub. No.: US 2024/0352452 A1
(43) Pub. Date: Oct. 24, 2024

(54) CRISPR-BASED PROTEIN BARCODING AND SURFACE ASSEMBLY

(71) Applicant: The Brigham and Women's Hospital, Inc., Boston, MA (US)

(72) Inventors: Stephen J. ELLEDGE, Boston, MA (US); Karl W. BARBER, Boston, MA (US)

(21) Appl. No.: 18/577,385

(22) PCT Filed: Jul. 11, 2022
(86) PCT No.: PCT/US2022/036736

§ 371 Date: Jan. 8, 2024

Related U.S. Application Data
(60) Provisional application No. 63/220,399, filed on Jul. 9, 2021.

(51) Int. Cl. C12N 15/10; C07K 17/02; C12N 9/22; C12N 15/11; C40B 40/10

(52) U.S. Cl. CPC C12N 15/1093; C07K 17/02; C12N 9/22; C12N 15/111; C40B 40/10; C12N 2310/15

(57) ABSTRACT

Biotechnological innovations have vastly improved the capacity to perform large-scale protein studies. The production and interrogation of custom protein libraries has proven important for a plethora of biological applications including multiplexed disease diagnostics, therapeutic antibody discovery, and directed evolution. The present invention relates to methods and compositions for use in making Cas-related fusion protein libraries barcoded with sgRNA sequences for applications in protein studies and for protein self-assembly on surfaces.

### CROSS-REFERENCE TO RELATED APPLICATION

This application claims benefit of U.S. Provisional Application No. 63/220,399, filed Jul. 9, 2021, the contents of which are incorporated herein by reference in their entirety.

### BACKGROUND

The invention relates to Cas-related fusion proteins and uses thereof.

Protein microarrays consist of a solid surface harboring thousands of immobilized proteins at spatially discrete positions and can be used to monitor biological samples for the presence of many disease-related biomolecules (Sutandy et al., Curr. Protoc. Protein Sci. 72, 27.1.1-27.1.16 (2013); Duarte et al., Expert Rev Proteomic 14, 627-641 (2017); Cretich et al., Analyst 139, 528-542 (2013)). These microarrays have been widely used for basic research applications including antibody epitope mapping, enzymatic activity profiling, and global protein interactomics studies, and the resulting binding and reactivity profiles can be informative in disease diagnostics (Hanash, Nature 422, 226-232 (2003); Hartmann et al., Anal Bioanal Chem 393, 1407-1416 (2009); Poetz, et al., Proteomics 5, 2402-2411 (2005)). Still, protein microarrays are generally expensive and laborious to construct, requiring the individual purification of each of thousands of proteins to be spotted on the microarray surface.

Other biotechnological innovations have also vastly improved the capacity to perform large-scale protein studies. The production and interrogation of custom protein libraries has proven important for a plethora of biological applications including multiplexed disease diagnostics, therapeutic antibody discovery, and directed evolution (Fernandez-Gacio et al., Trends Biotechnol. 21, 408-414, (2003); Hartmann et al., Anal Bioanal Chem 393, 1407-1416 (2009); Sidhu, 2000). These studies are often performed using in vitro protein display techniques such as phage and ribosome display, in which proteins are linked to unique nucleic acid barcodes (Xu et al., Science 348, aaa0698 (2015); Zhu et al., Nat Biotechnol, 31: 331-334, (2013)).

Accordingly, there still exists a need in the art for protein libraries that can be efficiently designed and customized, yet also overcome the existing labor and cost barriers of current protein microarrays and other protein display platforms.

### SUMMARY OF THE INVENTION

The invention, in general, features Cas-containing fusion proteins and methods of using the same.

In one aspect, the invention features a surface including (a) a nucleic acid molecule; and (b) a Cas-related protein including (i) a single guide RNA (sgRNA) and (ii) a fusion protein of interest.

In some embodiments of the foregoing aspect, the surface is a microarray or a non-microarray surface.

In another aspect, the invention features a composition including a Cas-related protein including (i) an sgRNA and (ii) a protein fusion of interest.

In some embodiments of either of the foregoing aspects, the nucleic molecule is DNA or RNA.

In some embodiments of either of the foregoing aspects, the Cas-related protein is a catalytically inactive Cas9, Cas12a, Cas13, or Cas14 protein.

In some embodiments of either of the foregoing aspects, the protein of interest is an epitope tag, a viral protein, a bacterial protein, a parasitic protein, or an animal protein. For example, in some embodiments, the epitope tag is HA, myc, FLAG, or 6His.

In some embodiments of a foregoing aspect, the composition includes a nucleic acid molecule, wherein said nucleic acid molecule binds sgRNA associated with the Cas-related protein.

In another aspect, the invention features a method for making a fusion protein library for use in a self-assembling protein microarray, the method including, for each member of the library, providing or making a Cas-containing fusion protein, wherein the Cas-containing fusion protein includes: (a) a catalytically inactive Cas-related protein; (b) a protein of interest, wherein the protein of interest is linked to the Cas-related protein; and (c) a sgRNA, wherein the sgRNA includes a unique nucleotide sequence complementary to a target sequence of a DNA probe.

In another aspect, the invention features a method for making a fusion protein for use in protein immobilization of a single protein on a non-microarray surface, the method including providing or making a Cas-containing fusion protein, wherein the Cas-containing fusion protein includes: (a) a catalytically inactive Cas-related protein; (b) a protein of interest, wherein the protein of interest is linked to the Cas-related protein; and (c) a sgRNA, wherein the sgRNA includes a unique nucleotide sequence complementary to a target sequence of a DNA probe.

In another aspect, the invention features a method for making a fusion protein library for use in protein immobilization on a non-microarray surface, the method including, for each member of the library, providing or making a Cas-containing fusion protein, wherein the Cas-containing fusion protein includes: (a) a catalytically inactive Cas-related protein; (b) a protein of interest, wherein the protein of interest is linked to the Cas-related protein; and (c) a sgRNA, wherein the sgRNA includes a unique nucleotide sequence complementary to a target sequence of a DNA probe.

In some embodiments of a preceding aspect, the method further includes causing the self-assembling protein microarray to self-assemble, the method including the steps of: (i) making or providing a surface to which a plurality of DNA probes is attached, wherein each DNA probe includes a target sequence; and (ii) contacting the plurality of DNA probes with the fusion protein library under conditions that allow the specific hybridization of each sgRNA with its complementary target sequence, thus immobilizing each Cas-containing fusion protein on the surface.

In some embodiments of any of the preceding aspects, making each Cas-containing fusion protein includes (i) making or providing a single plasmid including a nucleotide sequence encoding the Cas-containing fusion protein and a nucleotide sequence encoding the sgRNA; and (ii) causing the fusion protein and the sgRNA to be expressed and to assemble into a fusion protein-sgRNA complex.

In some embodiments of any of the preceding aspects, making each Cas-containing fusion protein includes (i) making or providing a pair of plasmids, wherein a first plasmid of the pair includes a nucleotide sequence encoding the Cas-containing fusion protein and a second plasmid of the pair includes a nucleotide sequence encoding the sgRNA; and (ii) causing the fusion protein and the sgRNA to be expressed and to assemble into a fusion protein-sgRNA complex.

In some embodiments of any of the preceding aspects, the plasmid or plasmids are included by a host cell.

In some embodiments of any of the preceding aspects, the method is performed in an in vitro reaction. In some embodiments of any of the preceding aspects, the in vitro reaction includes an emulsion step, and wherein an emulsion droplet of the emulsion step includes the fusion protein and the sgRNA.

In some embodiments of a preceding aspect, the fusion protein library includes at least two unique Cas-containing fusion proteins. For example, in some embodiments, the fusion protein library includes 100, 1,000, 10,000, 100,000, 125,000, 250,000, 500,000, 750,000, or 1,000,000 unique Cas-containing fusion proteins.

In some embodiments of any of the preceding aspects, the protein of interest is 8-40 amino acids in length. In some embodiments, the protein of interest is greater than 40 (e.g., 41, 42, 43, 44, 45, 50, 60, 70, 80, 90, 100, 500, 1,000, 1,500, and 2,000) amino acids in length.

In some embodiments of a preceding aspect, the method further includes contacting the protein microarray with a biological sample under conditions that would allow a specific reaction between a Cas-containing fusion protein of interest of the fusion protein library and a moiety in the biological sample.

In some embodiments of a preceding aspect, the method further includes contacting the non-microarray surface with a biological sample under conditions that would allow a specific reaction between a Cas-containing fusion protein of interest of the fusion protein library and a moiety in the biological sample.

In some embodiments of a preceding aspect, the non-microarray surface is a microbead, a wire, a smart material, a hydrogel, or any other suitable solid material.

In some embodiments of any of the preceding aspects, the sgRNA further includes a 5′ constant region located 5′ to the sgRNA spacer sequence. In some embodiments, the method further includes amplifying the sgRNA using the 5′ constant region located 5′ to the sgRNA spacer sequence using a sequencing-based method. For example, in some embodiments, the sequence-based method includes a polymerase chain reaction (PCR), a reverse transcription PCR, or nucleic acid sequencing (e.g., Sanger sequencing or next-generation sequencing).

In some embodiments of any of the preceding aspects, the method further includes identifying a reaction between a fusion protein of interest of the fusion protein library and a moiety in the biological sample by detecting a specific reaction.

In some embodiments of any of the preceding aspects, the reaction is an interaction.

In some embodiments of any of the preceding aspects, the protein of interest fused to the Cas-containing fusion protein is pathogen-associated. For example, in some embodiments, the pathogen-associated protein is a SARS-CoV-2 protein or a fragment thereof. In some embodiments, the pathogen-associated protein is a viral pathogen-associated protein. For example, in some embodiments, the viral pathogen-associated protein is a SARS-CoV-2 protein.

In some embodiments of any of the preceding aspects, the protein of interest included by the Cas-containing fusion protein corresponds to a protein or a fragment thereof in the proteome of an organism, for example, a bacterium, a virus, a fungus, an animal (for example, a human), a plant, or an invertebrate. In some embodiments, the protein of interest is synthetic. In some embodiments, the protein of interest included by the Cas-containing fusion protein is an antibody or an antibody-like protein or peptide.

In some embodiments of any of the preceding aspects, the moiety is an antibody or a disease biomarker. For example, in some embodiments, the antibody is an antiviral antibody. In some embodiments, the antiviral antibody is an anti-SARS-CoV-2 antibody.

In another aspect, the invention features a Cas-containing fusion protein library wherein each protein complex includes (a) a catalytically inactive Cas-related protein; (b) a protein of interest, wherein the protein of interest is linked to the Cas-related protein; and (c) a sgRNA, wherein the sgRNA includes a unique nucleotide sequence. In some embodiments, the sgRNA includes a unique sequence which is complementary to a target sequence of a DNA probe.

In some embodiments of any of the preceding aspects, the catalytically inactive Cas-related protein is a catalytically inactive Cas9, Cas12a, Cas13, or Cas14 protein. For example, in some embodiments, the catalytically inactive Cas9 protein is dCas9.

In some embodiments of any of the preceding aspects, the protein of interest is fused to the C terminus of the Cas-related protein. In some embodiments, the protein of interest is fused to the N terminus of the Cas-related protein.

In some embodiments of any of the preceding aspects, each DNA probe includes a 3′ universal annealing sequence; a target sequence, wherein the target sequence is complementary to an sgRNA spacer sequence; a protospacer adjacent motif (PAM) sequence; and a 5′ universal sequence. In some embodiments, each DNA probe includes the target sequence adjacent to the PAM sequence.

In some embodiments of any of the preceding aspects, the DNA probe is attached to a solid surface.

In some embodiments of any of the preceding aspects, the protein of interest is a viral protein or a fragment thereof. For example, in some embodiments, the viral protein is a SARS-CoV-2 protein or a fragment thereof. In some embodiments, the viral protein is a human immunodeficiency virus (HIV) protein, an influenza A protein, a hepatitis C protein, a coronaviruses like HKU1 protein, or an Ebola protein or a fragment thereof.

In some embodiments of any of the preceding aspects, each DNA probe is tethered to the support at its 3′ end. In some embodiments, each DNA probe is tethered to the support at its 5′ end.

In some embodiments of any of the preceding aspects, each DNA probe is single-stranded. In some embodiments, each DNA probe is partially or completely double-stranded.

In some embodiments of any of the preceding aspects, no two DNA probes share more than 50% sequence identity in the target sequence.

In some embodiments of any of the preceding aspects, the sgRNA spacer sequence has at least 50% sequence complementarity with the target sequence of any unique DNA probe.

In some embodiments of any of the preceding aspects, 6 or more bases in the DNA target sequence adjacent to the PAM motif are complementary to the bases on the 3′ end of the sgRNA spacer sequence.

In another aspect, the invention features a fusion protein library, the library including a plurality of Cas-containing fusion proteins, wherein each Cas-containing fusion protein includes: (a) a catalytically inactive Cas-related protein; (b) a protein of interest, wherein the protein of interest is linked to the Cas-related protein; and (c) a sgRNA, wherein the sgRNA includes a unique nucleotide sequence complementary to a target sequence of a DNA probe.

In another aspect, the invention features a plasmid library, the library including a plurality of plasmids encoding Cas-containing fusion proteins, wherein each plasmid encodes: (a) a catalytically inactive Cas-related protein; (b) a protein of interest, wherein the protein of interest is linked to the Cas-related protein; and (c) a sgRNA, wherein the sgRNA includes a unique nucleotide sequence complementary to a target sequence of a DNA probe.

In another aspect, the invention features a capture complex, the complex including: (i) a DNA probe, wherein the DNA probe includes a target sequence and is attached to a surface; and (ii) a Cas-containing fusion protein, wherein the Cas-containing fusion protein includes: (a) a catalytically inactive Cas-related protein; (b) a protein of interest, wherein the protein of interest is linked to the Cas-related protein; and (c) a sgRNA, wherein the sgRNA includes a unique nucleotide sequence complementary to the target sequence of a DNA probe; wherein the fusion protein is localized to the surface by base pairing interaction between the unique nucleotide sequence of the sgRNA and the target sequence of the DNA probe, thus forming the capture complex.

In some embodiments of any of the preceding aspects, the sgRNA further includes a 5′ constant region located 5′ to the sgRNA spacer sequence.

In another aspect, the invention features a composition including a host cell including a pair of plasmids, wherein a first plasmid of the pair includes a nucleotide sequence encoding a Cas-containing fusion protein and a second plasmid of the pair includes a nucleotide sequence encoding a sgRNA.

In some embodiments of any of the preceding aspects, the host cell is a bacterial cell, a mammalian cell, or a yeast cell. For example, in some embodiments, the bacterial cell is an E. coli cell.

In one aspect, the invention features, a method for making a fusion protein library, the method including, for each member of the library, providing or making a Cas-containing fusion protein, wherein the Cas-containing fusion protein includes:
(a) a catalytically inactive Cas-related protein;
(b) a protein of interest, wherein the protein of interest is linked to the Cas-related protein; and
(c) a single guide RNA (sgRNA), wherein the sgRNA includes a unique nucleotide sequence.

In some embodiments, the sgRNA is utilized for sgRNA sequencing.

In some embodiments, the sgRNA is complementary to a target sequence of a DNA probe.

In another aspect, the invention features a method for making a fusion protein for use in protein immobilization of a single protein on a non-microarray surface, the method including providing or making a Cas-containing fusion protein, wherein the Cas-containing fusion protein includes:
(a) a catalytically inactive Cas-related protein;
(b) a protein of interest, wherein the protein of interest is linked to the Cas-related protein; and
(c) a sgRNA, wherein the sgRNA includes a unique nucleotide sequence complementary to a target sequence of a DNA probe.

In another aspect, the invention features a method for making a fusion protein library for use in protein immobilization on a non-microarray surface, the method including, for each member of the library, providing or making a Cas-containing fusion protein, wherein the Cas-containing fusion protein includes:
(a) a catalytically inactive Cas-related protein;
(b) a protein of interest, wherein the protein of interest is linked to the Cas-related protein; and
(c) a sgRNA, wherein the sgRNA includes a unique nucleotide sequence complementary to a target sequence of a DNA probe.

In some embodiments, the aforementioned method further includes causing a self-assembling protein microarray to self-assemble, the method including the steps of:
(i) making or providing a surface to which a plurality of DNA probes is attached, wherein each DNA probe includes a target sequence; and
(ii) contacting the plurality of DNA probes with the fusion protein library under conditions that allow the specific hybridization of each sgRNA with its complementary target sequence, thus immobilizing each Cas-containing fusion protein on the surface.

In some embodiments, each DNA probe includes a 3′ universal annealing sequence; a target sequence, wherein the target sequence is complementary to an sgRNA spacer sequence; a protospacer adjacent motif (PAM) sequence; and a 5′ universal sequence.

In some embodiments, each DNA probe includes the target sequence adjacent to the PAM sequence.

In some embodiments, each DNA probe is attached to a solid surface.

In some embodiments, the sgRNA further includes a 5′ constant region or a primer annealing region located 5′ to the sgRNA spacer sequence.

In some embodiments of any of these aspects, making each Cas-containing fusion protein includes:
(i) making or providing a single plasmid including a nucleotide sequence encoding the Cas-containing fusion protein and a nucleotide sequence encoding the sgRNA; and
(ii) causing the fusion protein and the sgRNA to be expressed and to assemble into a fusion protein-sgRNA complex.

In some embodiments, the method is performed in vitro or in vivo (such as utilizing a plasmid or plasmids which are included by a host cell).

In some embodiments of any of these aspects, wherein making each Cas-containing fusion protein includes:
(i) making or providing a pair of plasmids, wherein a first plasmid of the pair includes a nucleotide sequence encoding the Cas-containing fusion protein and a second plasmid of the pair includes a nucleotide sequence encoding the sgRNA; and
(ii) causing the fusion protein and the sgRNA to be expressed and to assemble into a fusion protein-sgRNA complex.

In some embodiments, the method is performed in vitro.

In some embodiments, the plasmid or plasmids are included by a host cell.

In some embodiments, the host cell is a bacterial cell, a mammalian cell, or a yeast cell.

In some embodiments, the method further includes contacting the protein microarray with a sample (e.g., a biological sample) under conditions that would allow a specific reaction between a Cas-containing fusion protein of interest of the fusion protein library and a moiety in the sample.

In some embodiments, wherein the protein of interest fused with the Cas-containing fusion protein is pathogen-associated.

In some embodiments, wherein the protein of interest fused with the Cas-containing fusion protein corresponds to a protein or a fragment thereof in the proteome of an organism, for example, a bacterium, a virus, a fungus, an animal (for example, a human), a plant, or an invertebrate.

In some embodiments, wherein the protein of interest fused with the Cas-containing fusion protein is an antibody or an antibody-like protein or peptide.

In some embodiments, wherein the moiety is an antibody or a disease biomarker.

In some embodiments, the method further includes amplifying the sgRNA using the 5′ constant region or a primer annealing region located 5′ to the sgRNA spacer sequence using a sequencing-based method.

In some embodiments of any of these aspects, the method further includes identifying a reaction between a fusion protein of interest of the fusion protein library and a moiety in the sample by detecting a specific reaction.

In some embodiments, the protein of interest fused with the Cas-containing fusion protein is pathogen-associated.

In some embodiments, the protein of interest fused with the Cas-containing fusion protein corresponds to a protein or a fragment thereof in the proteome of an organism, for example, a bacterium, a virus, a fungus, an animal (for example, a human), a plant, or an invertebrate.

In some embodiments, the protein of interest fused with the Cas-containing fusion protein is an antibody or an antibody-like protein or peptide.

In some embodiments, the moiety is an antibody or a disease biomarker.

In still another aspect, the invention features a Cas-containing fusion protein library, wherein each member of the library includes:
(a) a catalytically inactive Cas-related protein;
(b) a protein of interest, wherein the protein of interest is linked to the Cas-related protein; and
(c) a sgRNA, wherein the sgRNA includes a unique nucleotide sequence

In some embodiments, each sgRNA is complementary to a target sequence of a DNA probe.

In some embodiments, wherein each Cas-containing fusion protein is in association with DNA probe on a surface.

In some embodiments, the sgRNA includes a 5′ primer annealing region.

In some embodiments, the surface contains a plurality of DNA probes, wherein no two DNA probes share more than 50% sequence identity within the sgRNA-complementary target sequence.

In some embodiments, the sgRNA spacer sequence has at least 50% sequence complementarity with the target sequence of any unique DNA probe.

In some embodiments, the sgRNA further includes a 5′ constant region or a primer annealing region located 5′ to the sgRNA spacer sequence.

In yet another aspect, the invention features a plasmid library, the library including a plurality of plasmids encoding Cas-containing fusion proteins, wherein each plasmid encodes:
(a) a catalytically inactive Cas-related protein;
(b) a protein of interest, wherein the protein of interest is linked to the Cas-related protein; and
(c) a sgRNA, wherein the sgRNA includes a unique nucleotide sequence complementary to a target sequence of a DNA probe.

In yet another aspect, the invention features a capture complex, the complex including:
(i) a DNA probe, wherein the DNA probe includes a target sequence; and
(ii) a Cas-containing fusion protein complex, wherein the Cas-containing fusion protein complex includes:
   (a) a catalytically inactive Cas-related protein;
   (b) a protein of interest, wherein the protein of interest is linked to the Cas-related protein; and
   (c) a sgRNA, wherein the sgRNA includes a unique nucleotide sequence.

In some embodiments, the DNA probe is attached to a surface.

In some embodiments, the sgRNA includes a unique nucleotide sequence complementary to the target DNA sequence of a DNA probe.

In some embodiments, the fusion protein is localized to the surface by base pairing interaction between the unique nucleotide sequence of the sgRNA and the target sequence of the DNA probe.

In still another aspect, the invention features a surface including:
(a) a nucleic acid molecule; and
(b) a Cas-related protein complex including (i) an sgRNA and (ii) a protein of interest, wherein the Cas-related protein is fused to the protein of interest which is bound to the sgRNA.

In some embodiments, the surface is a microarray or a non-microarray surface.

In some embodiments, wherein the protein of interest is a synthetic antibody, a pathogen-derived protein, a mammalian protein, or a mutant protein variant thereof of a pathogen derived protein or a mammalian protein.

### BRIEF DESCRIPTION OF THE DRAWINGS

FIG. 1A is an overview of oligonucleotide-templated protein localization by protein immobilization by clustered regularly interspaced short palindromic repeats (CRISPR) associated protein 9 (Cas9)-mediated self-organization (PICASSO or “CRISPR-based protein self-assembly on surfaces” or “CRISPR-based protein surface self-assembly”).

FIG. 1B is a schematic of PICASSO for the construction of a complex protein microarray from a single mixed pool of dead Cas9 (dCas9)-fusion proteins self-assembling on a double-stranded DNA (dsDNA) microarray.

FIG. 1C shows dCas9-hexa histidine (6His)+single guide RNA (sgRNA; sgRNA spacer: 5′-CCGUACCUAGAUACACUCAA-3′ (SEQ ID NO: 20)) purified from E. coli localized to positions containing anticipated target DNA sequences on a microarray. Off-target positions all contained same DNA sequence (spacer: 5′-PAM-ATGCGGAGGGTTTCTTTTAT-3′ (SEQ ID NO:43)). An anti-6His antibody was used for fluorescent labeling. Scale bar=100 μm.

FIG. 1D shows single base substitutions along the length of a target DNA sequence prevented dCas9-6His+sgRNA binding for substitutions closest to the protospacer adjacent motif (PAM; sgRNA spacer: 5′-AGAGACUGCCCGACACAUCU-3′ (SEQ ID NO:43)). Binding is anti-6His fluorescence value with background subtraction (intensity minus value of unmutated target DNA with no PAM) relative to a maximum value. Average values for duplicate DNA features are shown, and asterisks denote DNA sequences with a perfect match to sgRNA.

FIG. 1E shows double base substitutions (C⇔G or A⇔T transversions) to the same sequences as in FIG. 1D. Binding is anti-6His fluorescence value with background subtraction (intensity minus value of unmutated target DNA with no PAM) relative to maximum value. Average values for duplicate DNA features are shown.

FIG. 2A shows a two-plasmid system employed for co-expression of dCas9-epitope fusions and a unique sgRNA sequence in E. coli. After double transformation, cells were co-cultured and dCas9-epitope+sgRNA library members were copurified.

FIG. 2B is a design of a template DNA microarray containing targets of library sgRNAs and off-target DNA sequences.

FIG. 2C shows dCas9-epitope fusions localized to the DNA microarray, as shown by fluorescent antibody labeling and described in FIG. 2B. Scale bar=100 μm.

FIG. 3A shows the cloning strategy to generate a pooled vector library encoding thousands of unique peptide-sgRNA pairs compatible with.

FIG. 3B shows the dCas9-FLAG saturation mutagenesis library displayed by PICASSO and probed with an M2 anti-FLAG antibody. A anti-hemagglutinin (HA) antibody was used for normalization based on total protein levels. Data is presented for an average of 4 peptide replicates with unique sgRNAs, each in a technical triplicate. Asterisks denote the wildtype sequence.

FIG. 3C shows the dCas9-influenza A (IAV) immunodominant peptide saturation mutagenesis library displayed by PICASSO and probed with patient serum. An anti-6His antibody was used for normalization based on total protein levels. Data is presented for an average of 4 peptide replicates with unique sgRNAs, each in a technical triplicate. Asterisks denote the wildtype sequence. Grey boxes indicate no data.

FIG. 3D shows the comparison of serum antibody binding to a IAV saturation mutagenesis library, as evaluated by PICASSO and phage display (average values at each position within epitope). Phage display enrichment is defined as normalized read counts in the sample immunoprecipitation compared to no-serum controls. Best fit line with R² values from simple linear regression are shown.

FIG. 3E shows the epitope mapping experiments using PICASSO to identify linear antibody targets within Severe Acute Respiratory Syndrome Coronavirus 2 (SARS-CoV-2) proteins from convalescent coronavirus disease 2019 (COVID-19) patients. An anti-6His antibody was used for normalization based on total protein levels. Data is presented for the highest value of 4 peptide replicates with unique sgRNAs, each in technical triplicate. Black arrows denote peptides recognized in at least 3 tested convalescent serum samples using a detection threshold of anti-IgG/anti-6His>1.3.

FIG. 4A shows that the dCas9 binding to the microarray depended on the presence of a PAM in the target DNA sequence for two tested sgRNA sequences paired with different dCas9-epitope fusions (dCas9-HA+sgRNA spacer #1: 5′-CCGUACCUAGAUACACUCAA-3′ (SEQ ID NO: 21) complementary to DNA probe #1; dCas9-myc+sgRNA spacer #2: 5′-AUAAAAGAAACCCUCCGCAU-3′ (SEQ ID NO: 22) complementary to DNA probe #2). Fluorescent anti-HA staining shown on left, fluorescent anti-myc staining shown on right. DNA probe #1 (n=97), #2 (n=101), and no-PAM controls (n=3 each) contained perfect match target sequences corresponding to respective sgRNA. 5′-CCA-3′ was changed to 5′-CGA-3′ in no PAM control oligonucleotide sequences.

FIG. 4B shows that single base substitutions along the length of a target DNA sequence prevented dCas9-6His+sgRNA binding for substitutions closest to the PAM (sgRNA spacer #2: 5′-CCGUACCUAGAUACACUCAA-3′ (SEQ ID NO: 23); #3: 5′-CACUGUUUAACAAGCCCGUC-3′ (SEQ ID NO: 24); #4: 5′-UCCAUAGAUUUCUCCGUGAG-3′ (SEQ ID NO: 25)). Binding is anti-6His fluorescence values with background subtraction (intensity minus value of unmutated target DNA with no PAM) relative to maximum value. Average values for duplicate DNA features are shown, and asterisks denote DNA sequence with a perfect match to sgRNA.

FIG. 4C shows the positional average fluorescence values for individual base substitutions averaged for the 4 tested sgRNA sequences shown in FIG. 1D and FIG. 4B.

FIG. 4D shows the double base substitutions (C⇔G or A⇔T transversions) for same sequences as in FIG. 5B. Binding is anti-6His fluorescence values with background subtraction (intensity minus value of unmutated target DNA with no PAM) relative to maximum. Average values for duplicate DNA features are shown.

FIG. 4E shows the evaluation of the effects of multiple sequential base substitutions (C⇔G and A⇔T) along the length of the target DNA sequence (n=3 spot replicates). Binding is anti-6His fluorescence values with background subtraction (intensity minus value of unmutated target DNA with no PAM) relative to maximum shown. Error bars show standard deviation centered at mean.

FIG. 5A shows the relative fluorescence intensity values using indicated monoclonal antibodies on PICASSO microarrays after four-member dCas9-epitope+sgRNA library application. DNA probe #1 (n=97 spot replicates), #2 (n=101), #3 (n=48), #4 (n=95), and top 10 scoring off-target sequences (n=2) out of 611 tested off-target (OT) sequences shown.

FIG. 5B shows a schematic of large-scale paired peptide-sgRNA plasmid library preparation for complex dCas9-fusion+sgRNA library expression for PICASSO.

FIG. 5C shows the rank-ordered anti-FLAG-based fluorescence intensities of DNA features after application of a 168-member PICASSO library, of which 12 peptides were a FLAG control. Top 10 off-target (OT) DNA features shown (n=3 spot replicates for all DNA features).

FIG. 5D shows the replicate FLAG peptide values from c averaged and top 10 off-targets (OT) shown.

FIG. 5E shows the correlation between anti-FLAG and anti-6His-based fluorescence signals, demonstrating ability to co-label PICASSO libraries using a test antibody and an antibody targeting a universal epitope tag. All dCas9-peptide fusions also had a C-terminal 6His tag, as shown in FIG. 6B. Best fit line with R² values from simple linear regression shown.

FIG. 5F shows the anti-FLAG/anti-His signal normalized signals shown for on-target FLAG peptide localization (same target DNA sequences as in FIG. 6C, n=3 spot replicates for all DNA features).

FIG. 5G shows the mean fluorescent signals (n=3 spot replicates for all DNA features) for unnormalized and normalized on-target FLAG peptides from FIG. 6C and FIG. 6F, respectively. Error bars show standard deviation centered at the mean.

FIG. 6A shows that after application of the dCas9-FLAG saturation mutagenesis library, PICASSO microarrays were simultaneously treated with anti-FLAG and anti-HA antibodies to characterize anti-FLAG antibody binding and using anti-HA-based total protein normalization.

FIG. 6B shows the immobilization of dCas9-FLAG saturation mutagenesis library on DNA microarray including 4 off-target controls where dCas9 is not anticipated to localize. Fluorescence from anti-HA (universal epitope tag) staining shown. n=3 spot replicates for each DNA feature.

FIG. 6C shows the sequence logo demonstrating reidentification of the M2 antibody DYKxxDxx binding motif from PICASSO data (Schneider et al., Nucleic Acids Res 18, 6097-6100 (1990); Tareen et al., Bioinformatics 36, 2272-2274 (2019)). Positions containing important residues from the motif highlighted in light blue.

FIG. 7A shows the dCas9-IAV immunodominant peptide saturation mutagenesis library displayed by and probed with patient serum. Anti-6His antibody used for normalization based on total protein levels. Data presented for average of 4 peptide replicates with unique sgRNAs, each in technical triplicate. Asterisks denote wildtype sequence. Grey boxes indicate no data.

FIG. 7B shows the phage display experiments using IAV immunodominant peptide saturation mutagenesis library. Data presented for average enrichment of 2 independent experiments. Enrichment is defined as normalized read counts in the sample immunoprecipitation compared to no-serum controls. Asterisks denote wildtype sequence. Grey boxes indicate no data.

FIG. 7C shows the comparison of serum antibody binding to IAV saturation mutagenesis library as evaluated by PICASSO and phage display for patient #2 (average values at each position within epitope).

FIG. 7D shows the comparisons between PICASSO and phage display for individual IAV peptide variants, and between phage display replicates.

FIG. 7E shows the comparison between PICASSO results for individual IAV peptide variants between the two tested patient samples (top) or using phage display (bottom). Peptides with substitutions at position 4 and 7 shown in blue and orange, respectively. Best fit lines with R² values from simple linear regression shown.

FIG. 8A shows the design of a tiled peptide library for linear epitope discovery across the SARS-CoV-2 proteome. Peptide tiles were centered around the same amino acids as tiles within the VirScan 56mer SARS-CoV-2 library.

FIG. 8B shows the VirScan epitope mapping results from a previous study (Shrock et al., Science 370, eabd4250 (2020)) for same patient samples evaluated in FIG. 3E. Black arrows show peptides scoring in 3 or more patient samples with Z-score >3.5.

FIG. 9 shows a CasPlay library design and workflow. Customized peptide libraries are encoded in an oligonucleotide library, with each peptide sequence paired with a unique 20 bp nucleic acid sequence to be used as the gRNA barcode. The peptides are expressed in E. coli as fusions to dCas9 bound to unique gRNA barcode sequences and purified in a single batch. The gRNA sequences have a universal 5′ constant region to facilitate amplification by RT-PCR. This library is then used for immunoprecipitation experiments using human serum antibodies. Peptides bound to these antibodies are identified by nucleotide sequencing of the enriched gRNA barcodes.

FIGS. 10A-10C show CasPlay experiments to map an antibody epitope with single amino acid resolution. FIG. 10A shows a FLAG peptide (DYKDDDK (SEQ ID NO: 26)) saturation mutagenesis library was produced for CasPlay in which every possible single amino acid substitution was performed along the length of the FLAG epitope, and each variant peptide was associated with a unique gRNA barcode. FIG. 10B shows that a sequencing-based enrichment analysis of gRNA barcodes revealed critical amino acid positions to coordinate M2 antibody binding to the FLAG peptide epitope. Enrichment data averaged across 4 gRNA barcode replicates and two independent experimental replicates. FIG. 10C shows enrichment of the DYKxxDxx motif for antibody binding was identified by sequence logo (Schneider and Stephens, Nucleic Acids Res 18, 6097-6100, 1990; Tareen and Kinney, Bioinformatics 36, 2272-2274, 2019). FIG. 10D shows sequence enrichment of FLAG variant peptides by CasPlay performed comparably with PICASSO, the microarray-based strategy for studying custom dCas9-fusion peptide libraries with fluorescence imaging readout (Barber et al., Mol Cell 81, 3650-3658, 2021).

FIGS. 11A-11B show SARS-CoV-2 epitope mapping by CasPlay. FIG. 11A shows peptide library tiling the SARS-CoV-2 proteome (excluding ORF1ab) from a previous study (Barber et al., 2021) was presented by CasPlay. FIG. 11B shows CasPlay experiments were performed using serum samples from 8 samples collected prior to December 2020 (“pre-COVID”), 8 patients following COVID-19 infection (“convalescent”), 8 individuals prior to receiving a vaccine, and the same 8 individuals between two weeks and three months after receiving the second dose of an mRNA vaccine. gRNA barcode enrichment analysis by CasPlay revealed epitope regions in the spike and/or nucleocapsid proteins recognized by patient antibodies in convalescent and vaccinated patient samples. Enrichment data averaged across 4 gRNA barcode replicates and two independent experimental replicates.

FIGS. 12A-12C show CasPlay enables interrogation of antibodies on human virome scale. FIG. 12A shows a 122,501-member tiled peptide-based representation of the human virome displayed by CasPlay based on peptide libraries from previous studies (Shrock et al., Science 370, eabd4250, 2020; Xu et al., Science 348, aaa0698, 2015). Each peptide was paired with an orthogonal gRNA barcode, with two separate barcodes assigned to each peptides (245,002 library members total). Epitope tags recognized by commercial monoclonal antibodies (e.g. FLAG, HA, myc) were also encoded as control peptides within the library. FIG. 12B shows CasPlay control experiments using anti-FLAG, anti-HA, and anti-myc antibodies identified all ten corresponding control replicates and none of the off-target epitope tag peptides, within the context of the entire 245,002-member library. FIG. 12C shows CasPlay was able to identify subregions of viral proteins commonly targeted by multiple patients (public epitopes) from within the 245,002-member library. Examples shown from adenovirus A, Epstein-Barr virus, and RSV. Z-scores were averaged for gRNA barcode duplicates across two independent patient samples.

FIGS. 13A-13C show CasPlay is compatible with full-length protein display and applications. FIG. 13A shows synthetic antibodies were expressed as C-terminal fusions to dCas9 with unique gRNA barcodes. A nanobody recognizing a peptide from β-catenin (Braun et al., Sci Rep-Uk 6, 19211, 2016; Traenkle et al., Mol Cell Proteomics 14, 707-723, 2015) and an scFv recognized the spike protein from SARS-CoV-2 (Wang et al., Science 373, 2021) were paired with unique gRNAs. FIG. 13B shows that target antigens of the synthetic antibodies were adsorbed to a plate surface and then incubated with the dCas9-antibody fusions. Primers specific to the gRNA sequences were then used to RT-PCR the gRNAs associated with each antibody to detect binding of the antibody to its target antigen. FIG. 13C shows densitometry of RT-PCR amplicons on an agarose gel reveal specific retention and detection of each synthetic antibody only in the presence of its respective analyte, indicating synthetic antibody functionality. n=3 independent replicates for each condition. Mean with standard deviation plotted.

FIG. 14 shows detailed CasPlay plasmid library cloning overview. Schematic overview of CasPlay oligonucleotide library DNA sequence features and library cloning methods.

FIG. 15 shows cross-platform comparison of SARS-CoV-2 epitope mapping. Matched patient sample data (6 convalescent patient serum samples, 5 pre-COVID serum samples) using tiled peptides representing SARS-CoV-2 shown. PICASSO and VirScan data obtained in and adapted from Barber et al., Mol Cell 81, 3650-3658, 2021 (Barber et al., Mol Cell 81, 3650-3658, 2021). Black arrowheads indicate epitopes recognized by two or more patients in this dataset above signal thresholds (CasPlay enrichment ≥3.5, PICASSO signal ratio ≥1.3, VirScan z-score ≥3.5). White arrowhead indicates epitope identified only by VirScan. Red arrowhead indicates epitope identified in both CasPlay and VirScan but not PICASSO, but only in one patient in this set.

FIGS. 16A-16F show CasPlay human virome library characterization and control experiments. FIG. 16A a sequencing read distribution of 245,002 barcodes in final plasmid library encoding dCas9-peptide fusion and gRNA pairs. FIG. 16B a sequencing read distribution of 245,002 barcodes from RT-PCR of gRNAs from final purified dCas9-fusion library preparations. FIG. 16C shows that there was no correlation between gRNA barcode read counts in final plasmid library and purified protein library. FIG. 16D shows that there was a correlation between independent sample replicates (from two separate transformations of the plasmid library) of gRNA barcodes sequenced from purified protein library. FIG. 16E shows top 500 enriched peptides from the 245,002-member CasPlay library experiments using anti-FLAG, anti-HA or anti-myc antibody ranked by z-score of corresponding gRNA barcode. Epitope tag replicates corresponding to the designated antibody indicated by star symbols. Peptide z-scores averaged for two independent sample replicates. All expected control peptides are plotted, with the exception of one FLAG peptide which ranked #525 in the anti-FLAG immunoprecipitation. FIG. 16F shows a comparison of CasPlay z-scores for the FLAG control peptides in the anti-FLAG immunoprecipitation experiment and the barcode read counts in the 245,002-member CasPlay input library, averaged for two independent sample replicates.

FIGS. 17A-17B show the reproducibility of CasPlay virome results. FIG. 17A shows an example of stability of measured antibody repertoire by CasPlay demonstrated by number of peptide hits (z-score ≥3.5, left) or peptide hits as a fraction of the size of a given viral proteome (right) in the CasPlay library from two patient-matched longitudinal samples, taken between two weeks and three months apart. FIG. 17B shows an example of inconsistency of peptide hits as total peptide hits (z-score ≥3.5, left) or peptide hits as fraction of size of corresponding viral proteome (right) for two unrelated samples.

FIGS. 18A-18D show the comparison of CasPlay to VirScan, FIG. 18A shows public epitopes from adenovirus C penton protein and EBV EBNA1 protein previously discovered by VirScan (Xu et al., 2015) were also uncovered by CasPlay (z-score ≥3.5). z-scores for each peptide from the annotated protein were averaged for 30 patient serum samples (with no suppositions about previous viral infection or vaccination status), measured in duplicate. FIG. 18B shows an average number of peptides scoring per virus (for peptide z-score ≥3.5) across all 30 tested patient samples, compared between VirScan and CasPlay. FIG. 18C shows a correlation of peptide z-scores for representative public epitopes from 30 patient serum samples plotted individually, measured in duplicate. FIG. 18D shows a correlation of z-scores for each peptide derived from individual viruses or the entire virome averaged for 30 patient samples, compared between CasPlay and VirScan.

FIG. 19 shows agarose gel images. Unprocessed agarose gel images from FIG. 13.

FIGS. 20A-20B show PICASSO-based application of dCas9-full length protein fusions. FIG. 20A shows a strategy for presenting a dCas9-fusion protein via PICASSO, adapted from Barber, et al., Mol Cell 81, 3650-3658, 2021 (Barber et al., Mol Cell 81, 3650-3658, 2021). FIG. 20B shows a dCas9-scFv fusion (anti-spike B1-182.1) presented via PICASSO enables fluorescence-based detection of corresponding analyte (spike protein). n=6 replicate microarray features per condition.

Other features and advantages of the invention will be apparent from the following Detailed Description and the Claims.

### Definitions

Before describing the invention in detail, it is to be understood that this invention is not limited to particular compositions or biological systems, which can, of course, vary. It is also to be understood that the terminology used herein is for the purpose of describing particular embodiments only and is not intended to be limiting.

As used in this specification and the appended claims, the singular forms “a”, “an” and “the” include plural referents unless the content clearly dictates otherwise. Thus, for example, reference to “a DNA probe” optionally includes a combination of two or more such DNA probes, and the like.

It is understood that aspects and embodiments of the invention described herein include “comprising,” “consisting,” and “consisting essentially of” aspects and embodiments.

As used herein, the term “self-assembling protein” refers to a catalytically inactive Cas-related protein (e.g., dCas9), which includes a single guide RNA (sgRNA) and a fused protein of interest that localizes to a position on a surface (e.g., a microarray surface or a non-microarray surface) containing a nucleic sequence (e.g., a DNA sequence) that is complementary to the self-assembling protein's associated sgRNA. As used herein, self-assembling proteins typically do not require manual spotting at a position on a surface (e.g., a microarray surface or a non-microarray surface), but rather self-organize from mixed pools on customizable, template DNA surfaces (e.g., a template DNA microarray surface or a template DNA non-microarray surface).

The terms “catalytically inactive Cas-related protein,” “dead Cas,” and “dCas” (e.g., dCas9) refer, interchangeably, to a nuclease-deficient variant of a Cas nuclease that retains its ability to bind to a nucleic acid (e.g., DNA through sgRNA:DNA base pairing using dCas9, dCas12a, or dCas14; or RNA through sgNA:RNA base pairing using dCas13); however, unlike a wild type Cas nuclease, where permanent gene disruption can be achieved, a nuclease-deficient variant of a Cas-related protein fails to generally introduce any genome modifications and lacks appreciable enzymatic activity. As used herein, exemplary “catalytically inactive Cas-related proteins” include but are not limited to dCas9, dCas12a, dCas13, and dCas14.

As used herein, the term “protein” refers to a polymer of amino acid residues (natural or unnatural) linked together most often by peptide bonds. The term, as used herein, refers to proteins, polypeptides, and peptides of greater than two amino acids in length, of any structure, and/or of any function. Polypeptides can include gene products, naturally occurring polypeptides, synthetic polypeptides, homologs, orthologs, paralogs, fragments and other equivalents, variants, and analogs of the foregoing. A polypeptide can be a single molecule or may be a multi-molecular complex such as a dimer, trimer, or tetramer. Most commonly disulfide linkages are found in multichain polypeptides. The term polypeptide can also apply to amino acid polymers in which one or more amino acid residues are an artificial chemical analogue of a corresponding naturally occurring amino acid. As used herein, the “length” of a protein refers to the linear size of the protein as assessed by measuring the quantity of amino acids from the 5′ to the 3′ end of the protein. Exemplary molecular biology techniques that may be used to determine the length of a protein of interest are known in the art.

As used herein, the term “protein of interest” refers to any protein to be analyzed, monitored, or screened. Exemplary proteins of interest include, but are not limited to, epitope tags (e.g. 6His, FLAG, HA, and myc), viral proteins (e.g., influenza A proteins, SARS-CoV-2 proteins, human immunodeficiency virus proteins, hepatitis C proteins, coronaviruses like HKU1 proteins, and Ebola proteins), mutated variants and fragments of viral proteins, bacterial proteins (e.g., E. coli proteins and salmonella proteins), parasitic proteins (e.g., Plasmodium falciparum proteins), animal proteins (e.g. mouse proteins, rat proteins, and human proteins (e.g., muscle-specific tyrosine kinase and acetylcholine receptors)). As is described herein, a protein of interest is typically fused to a Cas-related protein (e.g., Cas9, Cas12a, Cas13, Cas14, dCas9, dCas12a, dCas13, and dCas14) and associated (e.g., bound) with a unique sgRNA. For example, the Cas-related protein is noncovalently bound to the sgRNA.

As used herein, the terms “single guide RNA” and “sgRNA” refer to an RNA molecule that facilitates targeting of a Cas-related protein described herein (e.g., Cas9, Cas12a, Cas13, Cas14, dCas9, dCas12a, dCas13, and dCas14) to a target sequence. For example, a sgRNA can be a molecule that recognizes (e.g., hybridizes to) a target nucleic acid. An sgRNA is typically designed to be complementary to a target sequence. In some embodiments, the sgRNA is engineered to include a chemical or biochemical modification. In some embodiments, a sgRNA may include one or more nucleotides.

The term “capture complex” refers to an immobilized DNA molecule bound by a Cas-related fusion protein (e.g., a dCas9-fusion protein, a dCas12a-fusion protein, a dCas13-fusion protein, or a dCas14-fusion protein) via base pairing with an associated sgRNA.

As used herein, the term “target sequence” refers to a nucleic acid to which a targeting moiety (e.g., a spacer or a PAM motif) specifically binds. For example, the “target sequence” refers to a nucleic acid molecule (e.g., a DNA molecule) that is able to be bound by a Cas-related protein (e.g., a dCas9-fusion protein), e.g., targeted by virtue of complementarity between the PAM-adjacent DNA sequence and the spacer sequence of a sgRNA.

As used herein, the term “spacer” refers to an approximately 20 base pair DNA sequence (e.g., 15, 16, 17, 18, 19, 20, 21, 22, 23, 24, or 25 base pairs) that is adjacent to a PAM motif. The spacer, in general, shares the same sequence as the spacer sequence of the sgRNA. The sgRNA anneals to the complement of the spacer sequence on the target sequence.

As used herein, the terms “protospacer adjacent sequence,” “PAM,” and “PAM motif” refer to an approximately 2-6 base pair DNA sequence which serve as a targeting component of a Cas-related protein. Different PAM motifs can be associated with different Cas-related proteins (e.g., dCas9, dCas12a, dCas13, and dCas14) or equivalent proteins from different organisms. In addition, any given Cas-related protein may be modified to alter the PAM specificity of the Cas-related protein such that the Cas-related protein recognizes an alternative PAM motif. It will also be appreciated that Cas-related proteins from different bacterial species (e.g., orthologs) can have varying PAM specificities.

As used herein, the term “5′ constant region” refers to a sequence fused to the 5′ end of an sgRNA, for example, between the T7 promoter and SpeI site. As used herein, an exemplary 5′ constant region is 5′-AGATCAGGTACAGACTACGT-3 (SEQ ID NO: 27)’. 5′ constant regions, in some embodiments, may enable a sequencing-based readout (e.g., a polymerase chain reaction) of an sgRNA.

As used herein, “a primer annealing region” which is typically located 5′ to the sgRNA spacer sequence refers to a region within the sgRNA sequences that can be used for primer annealing and sequence amplification during reverse transcription PCR.

A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” As used herein, the term “base-pairing” refers to the formation of a stable duplex of nucleic acids by way of hybridization mediated by inter-strand hydrogen bonding according to Watson-Crick base pairing. The nucleic acids of the duplex may be, for example, at least 50% complementary to one another (e.g., about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% complementary to one another).

As used herein, the terms “hybridize” or “hybridization” refers to the formation of a stable duplex of nucleic acids by way of annealing mediated by inter-strand hydrogen bonding, for example, according to Watson-Crick base pairing. As used herein, the term “specific hybridization” refers to instances in which the nucleic acids of the duplex are at least 50% complementary to one another (e.g., about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% complementary to one another) or instances in which 6 or more bases in the DNA target sequence that are adjacent to the PAM motif are complementary to the bases on the 3′ end of an sgRNA spacer sequence.

“Percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:

100 multiplied by (the fraction X/Y)
where X is the number of complementary base pairs in an alignment (e.g., as executed by computer software, such as BLAST) in that program's alignment of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

The term “conditions that allow specific hybridization” as used herein, refers to conditions, which may include, for example, temperature, buffer compositions (e.g., salt concentrations), the concentration of a sample and/or a protein, and the time of a reaction which allow a target sequence or a portion thereof that need not be fully complementary (e.g., 100% complementary) to a sgRNA that has one or more nucleotide mismatches relative to the target sequence to hybridize to the target sequence. The “stable duplex” formed upon the specific hybridization of one nucleic acid to another is a duplex structure that is not denatured by a stringent wash. Exemplary stringent wash conditions are known in the art and include temperatures of about 5° C. less than the melting temperature of an individual strand of the duplex and low concentrations of monovalent salts, such as monovalent salt concentrations (e.g., NaCl concentrations) of less than 0.2 M (e.g., 0.2 M, 0.19 M, 0.18 M, 0.17 M, 0.16 M, 0.15 M, 0.14 M, 0.13 M, 0.12 M, 0.11 M, 0.1 M, 0.09 M, 0.08 M, 0.07 M, 0.06 M, 0.05 M, 0.04 M, 0.03 M, 0.02 M, 0.01 M, or less). The complementarity of the nucleic acids of the duplex may be low overall (e.g., less than 95%, less than 90%, less than 85%, less than 80%, less than 70%, less than 60%) but there may be segments of the nucleic acid that are contiguous and fully complementary to an equal-length segment of the target sequence that, in the duplex form, allow for hybridizing across the target sequence's length (e.g., the overall complementarity may be low, but there may be segments of at least 6 contiguous nucleotides, at least 7 contiguous nucleotides, at least 8 contiguous nucleotides, at least 9 contiguous nucleotides, or at least 10 contiguous nucleotides) that are fully complementarity to an equal-length segment of the target sequence, thus facilitating hybridization across the target sequence's length.

As used herein, a “non-microarray surface” refers to any solid support on which target sequences (e.g., a nucleic acid sequence e.g., a DNA sequence or an RNA sequence) can be immobilized for subsequent localization of a Cas-related protein (e.g., a dCas9-fusion protein localized to a DNA sequence or a dCas13-fusion protein localized to an RNA sequence). Exemplary non-microarray surfaces include any functionalized surface (e.g., a surface with covalent or noncovalent fusions of a reactive or adhesive chemical group) that enables a nucleic acid sequence to be attached to the surface, such as a functionalized hydrogel or a microbead. Additional examples of a non-microarray surface, include but are not limited to a wire or a smart material (e.g., a volume-responsive hydrogel permits detection of a biomolecule via changes in the volume of the hydrogel). As incorporated herein by reference, a smart material may include any material described by Guo et al. Smart Materials in Medicine, (2020). The nucleic acid sequence may need to contain a chemical modification for attachment to the non-microarray surface. For example, the nucleic acid (e.g., DNA) modifications may include the modification or incorporation of amino groups, biotinylation, thiol, or alkynes. The non-microarray surface may be made of any solid material, including, for example, glass, silicon, or polystyrene. The non-microarray surface may be planar or curved. A Cas-related protein localized onto a non-microarray surface may allow the subsequent detection of biomolecules (e.g., such as antibodies), for example, by fluorogenic methods.

As used herein, a “microarray surface” refers to a planar surface, a surface containing microwells, or, for example, any other surface with spatially arrayed nucleic acid sequences.

As used herein, “sample” refers to any mixture containing one or more analytes of interest, such as proteins, antibodies, or small molecules. A sample can be, for example, a biological sample obtained from a subject (e.g., a mammal, preferably a human). Exemplary biological samples that may be used include, without limitation, blood, peripheral blood, a blood component (e.g., serum, isolated blood cells, or plasma), buccal samples (e.g., buccal swabs), nasal samples (e.g., nasal swabs), urine, fecal material, saliva, amniotic fluid, cerebrospinal fluid (CSF), synovial fluid, tissue (e.g., from a biopsy), pancreatic fluid, chorionic villus sample, cells, extracellular matrix, cultured cells (prokaryotic or eukaryotic), cell lysates, cellular organelles, cancerous cells, or any combination or derivative thereof. In certain embodiments, a biological sample is purified recombinant protein or mixture of recombinant proteins. In certain embodiments, the biological sample is or includes blood. In certain embodiments, the biological sample includes a clinical sample (i.e., a sample obtained from a subject). Furthermore, a sample can be processed (e.g., washed) prior to testing in the methods of the invention. Alternatively, the sample can be an unprocessed sample. Detection of analytes can be for noncovalent or covalent interaction

### DETAILED DESCRIPTION

We have developed a clustered regularly interspaced short palindromic repeats (CRISPR)-based system for facile custom protein microarray fabrication. The Cas9 nuclease from Streptococcus pyogenes has been previously deployed for many DNA editing applications (Doudna et al., Science 346, 1258096 (2014)). Catalytically inactive dead Cas9 (dCas9) is able to identify a specific genomic locus complementary to the spacer region of a complexed single guide RNA (sgRNA) followed by a protospacer adjacent motif (PAM), which facilitates dCas9 binding. When perfect complementarity exists between the sgRNA and target locus, dCas9 binds DNA virtually irreversibly at room temperature (e.g., see Boyle et al., Proc National Acad Sci 114, 5461-5466 (2017); Sternberg et al., Nature 507, 62-67 (2014)).

As is detailed below, we introduce protein immobilization by Cas9-mediated self-organization (PICASSO) to efficiently generate high-throughput oligonucleotide-templated programmable protein microarrays. This invention is based, at least in part, upon our demonstration that bespoke protein libraries fused to catalytically inactive Cas9 (dCas9) and coupled with unique single guide RNA (sgRNA) molecules rapidly self-assemble to user-defined positions on a DNA microarray surface, thereby enabling multiplexed protein assays. We generated dCas9-displayed saturation mutagenesis peptide microarrays by PICASSO to characterize antibody-epitope binding for a commercial anti-FLAG monoclonal antibody and human serum antibodies. Using Severe Acute Respiratory Syndrome Coronavirus 2 (SARS-CoV-2), as an example, we also show that PICASSO can be used for viral epitope mapping and exhibits promise as a multiplexed diagnostics tool. PICASSO is the first demonstration of a CRISPR-based protein display as well as complex protein library self-assembly using dCas9. This platform enables rapid interrogation of varied customized protein libraries or biological materials assembly using DNA scaffolding.

To facilitate the study of custom protein libraries and overcome the limitations of existing display technologies, we leveraged the properties of CRISPR systems to create a new in vitro protein display platform. By fusing recombinant proteins to dCas9, we were able to barcode protein libraries with unique identifier sgRNA barcode sequences. Then, using a technique we call protein immobilization by Cas9-mediated self-organization (PICASSO), the single mixed pool of dCas9-fusion proteins is able to localize to user-programmed positions on a microarray surface containing DNA sequences complementary to each protein's sgRNA barcode. The resulting DNA-templated self-assembling protein microarrays can be used for rapid large-scale protein studies. dCas9-fusion protein display and self-assembling microarray construction via PICASSO circumvent many of the caveats of other display platforms, making custom protein library studies faster and more broadly accessible. Therefore, this invention is based, at least in part, on the discovery that PICASSO offers unique advantages over other protein microarray fabrication techniques.

### EXAMPLE 1

dCas9-Based Protein Immobilization on a DNA Microarray by PICASSO

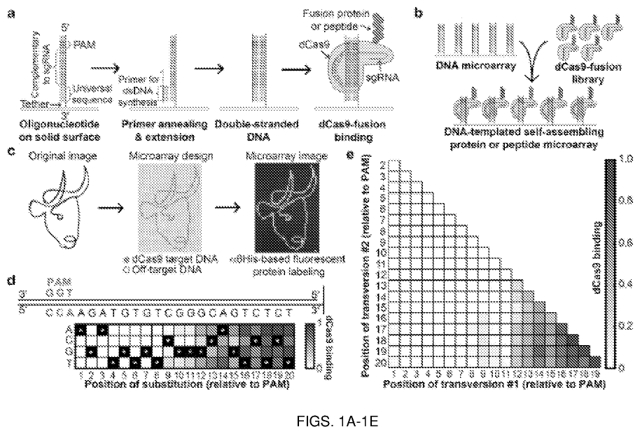

Since dCas9 tolerates a variety of C-terminal fusions with no effect on its DNA binding properties (e.g., see Chavez et al., Nat Methods 12, 326-328 (2015); Bikard et al., Nucleic Acids Res 41, 7429-7437 (2013)), we linked dCas9 to other proteins for immobilization on an oligonucleotide-based microarray, thereby creating a new class of DNA-templated protein microarray. Phosphoramidite-based oligonucleotide synthesis is a prevalent and cost-effective technique to generate single-stranded DNA (ssDNA) microarrays (e.g., see LeProust et al., Nucleic Acids Res 38, 2522-2540 (2010); Kosuri et al., Nat Biotechnol28, 1295-1299 (2010)). On the solid microarray surface, we designed oligonucleotides containing a universal primer hybridization site followed by a sequence complementary to a unique sgRNA and a PAM (FIG. 1A). By annealing a primer and using DNA polymerase for extension, the immobilized ssDNA probes can be made double stranded (e.g., see Bulyk et al., Nat Biotechnol 17, 573-577 (1999)). When a dCas9-fusion protein complexed with a unique sgRNA is introduced, the protein localizes to the corresponding complementary DNA probe. In this fashion, an oligonucleotide microarray with thousands of unique sequences can be used to generate a complex self-assembling PICASSO microarray (FIG. 1B).

Characterization of dCas9+sqRNA-DNA Binding on DNA Microarray

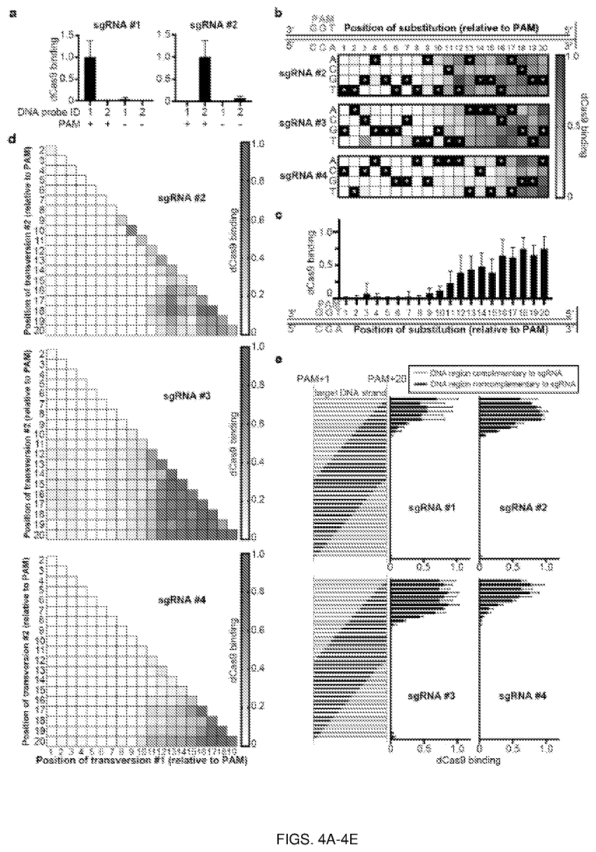

We introduced dCas9-hexa histidine (6His) complexed with a single sgRNA onto a dsDNA microarray. dCas9-6His localized to the anticipated positions on the dsDNA microarray surface containing DNA sequences complementary to the sgRNA (FIG. 1C). Consistent with other CRISPR-based systems, dCas9 binding exhibited PAM dependency on the microarray surface (FIG. 4A).

We realized that the possible diversity of proteins featured on PICASSO microarrays could be limited by off-target dCas9-fusion localization. To assess the theoretical complexity of dCas9-fusion protein libraries that could be displayed using PICASSO, we performed base substitutions in the target DNA probes and evaluated their impact on dCas9 binding. Single base substitutions within the region proximal to the PAM (known as the seed region) ablated dCas9 binding, reducing localization by more than 90% on average for substitutions within 9 bases of the PAM for four tested sgRNAs (FIG. 1D, FIG. 4B-4C). Double substitutions along the DNA target (FIG. 1E, FIG. 4D) or multiple substitutions within PAM-distal bases (FIG. 4E) further reduced dCas9 binding. These results suggest that PICASSO microarrays featuring hundreds of thousands of proteins with minimal off-target localization is possible. Our subsequent synthetic sgRNA and paired DNA probe design strategy ensured that no two sequences were perfect matches within the seed region, and we derived the remaining bases from a previously described list of orthogonalized DNA barcodes (e.g., see Xu et al., Proc National Acad Sci 106, 2289-2294 (2009)).

Demonstration of dCas9-Based Protein Library Self-Assembly on a DNA Microarray by PICASSO

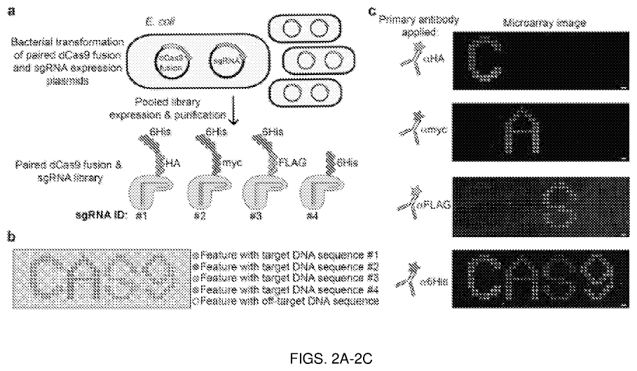

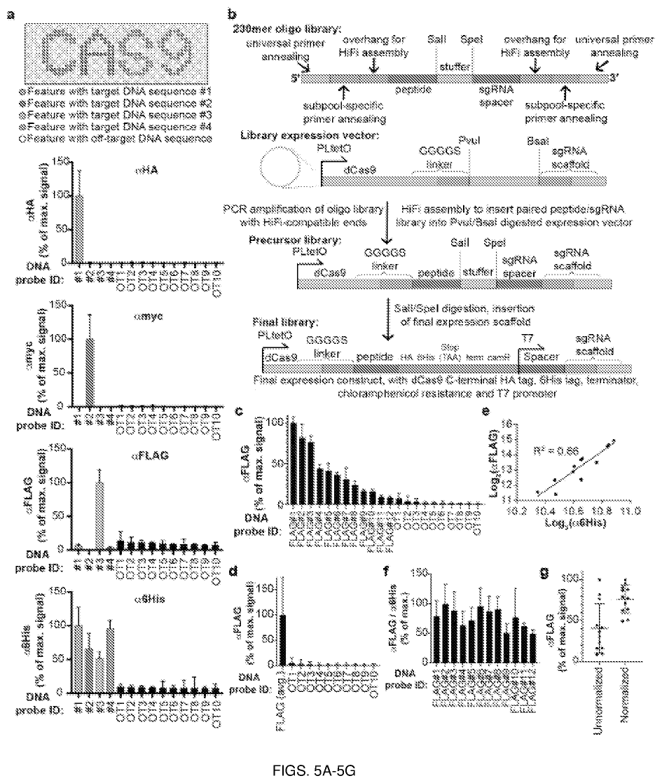

To demonstrate that PICASSO is compatible with multiplexed protein assembly, we co-expressed and copurified four different dCas9-epitope+sgRNA pairs in a single batch of E. coli (FIG. 2A). We designed a corresponding DNA microarray containing target sequences for each sgRNA in addition to >600 off-target probes (FIG. 2B). Each dCas9-epitope fusion assembled to its expected positions with low off-target binding as observed by fluorescent antibody labeling (FIG. 2C, FIG. 5A). Importantly, this result indicates that sgRNA exchange does not substantially occur between dCas9 molecules in the library, consistent with the picomolar dissociation constant between Cas9 and sgRNAs (e.g., see Wright et al., Proc National Acad Sci 112, 2984-2989 (2015)).

### PICASSO Microarray Development Using Complex Peptide Libraries for Antibody Detection Applications

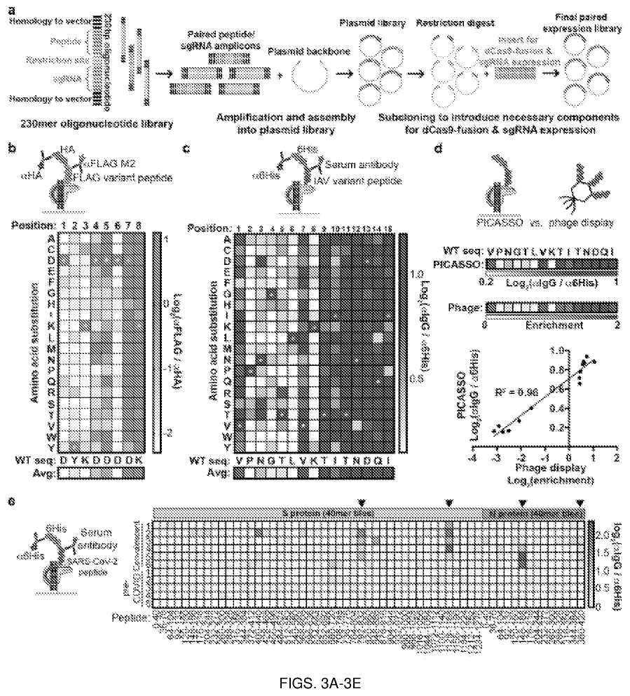

To generate complex libraries for PICASSO, we designed synthetic oligonucleotides for plasmid library construction encoding both a peptide of interest and a paired sgRNA on the same strand of DNA (FIG. 3A, FIG. 5B). To demonstrate the effectiveness of this library construction strategy for protein microarray self-assembly, we made a plasmid library encoding 154 dCas9-peptide fusions paired with unique sgRNAs including 12 replicates of a FLAG peptide control and constructed a corresponding DNA microarray containing anticipated dCas9 targets. After dCas9-peptide fusion library synthesis, purification, and application to the dsDNA microarray, the microarray was labeled with an anti-FLAG antibody. The 12 top-scoring DNA targets corresponded to all 12 of the on-target sequences where dCas9-FLAG was anticipated to localize (FIG. 5C). However, the FLAG peptide replicates paired with unique sgRNAs exhibited considerable variability in immobilization efficiency. To circumvent this, we implemented peptide replicate averaging to stratify on-target vs. off-target localization (FIG. 5D) and fluorescent co-staining of a universal epitope tag fused to each library member for total protein-based normalization. Together, this strategy reduced the standard deviation by about 44% for on-target FLAG peptides (FIG. 5E-5G).

### FLAG Peptide Saturation Mutagenesis Libraries and Antibody Characterization by PICASSO

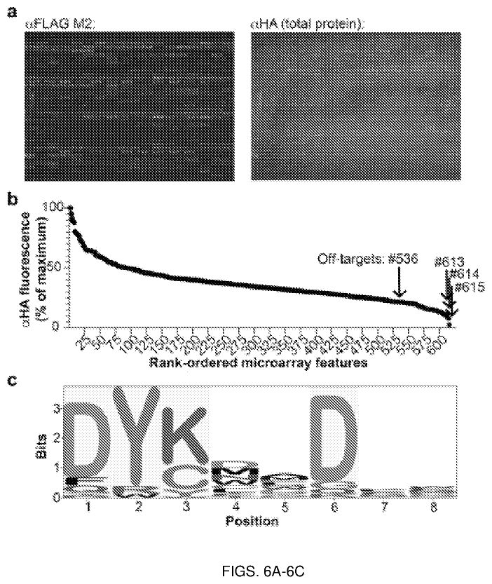

To benchmark PICASSO's performance for antibody binding studies, we generated a dCas9-linked peptide saturation mutagenesis library for the FLAG epitope, DYKDDDDK, and used it with the anti-FLAG M2 antibody. The 153 dCas9-FLAG peptide variants were encoded in quadruplicate paired with unique sgRNA sequences (612 peptide-sgRNA pairs total) with each peptide followed by a universal C-terminal hemagglutinin (HA) tag. We added the purified dCas9-fusion library to a corresponding DNA microarray and applied anti-HA and anti-FLAG M2 antibodies (FIG. 6A). Anti-HA (total protein) staining demonstrated that dCas9 library members localized to DNA features complementary to the encoded sgRNAs, with off-target features (i.e. DNA probes not complementary to any library sgRNAs) all scoring in the bottom 15% (FIG. 6B). Using the ratio of anti-FLAG:anti-HA signals and averaging results for each FLAG peptide variant across replicates, we observed a DYKxxDxx binding motif with mixed binding dependencies at the fourth and fifth positions (FIG. 3B, FIG. 6C), as has been seen previously for the M2 antibody using a DNA sequencing display method (e.g., see Layton et al., Mol Cell 73, 1075-1082.e4 (2019)), Together, these results confirm the utility of PICASSO for antibody-epitope binding characterization.

IAV Immunodominant Epitope Saturation Mutagenesis Experiments with PICASSO for Serum Antibody Characterization

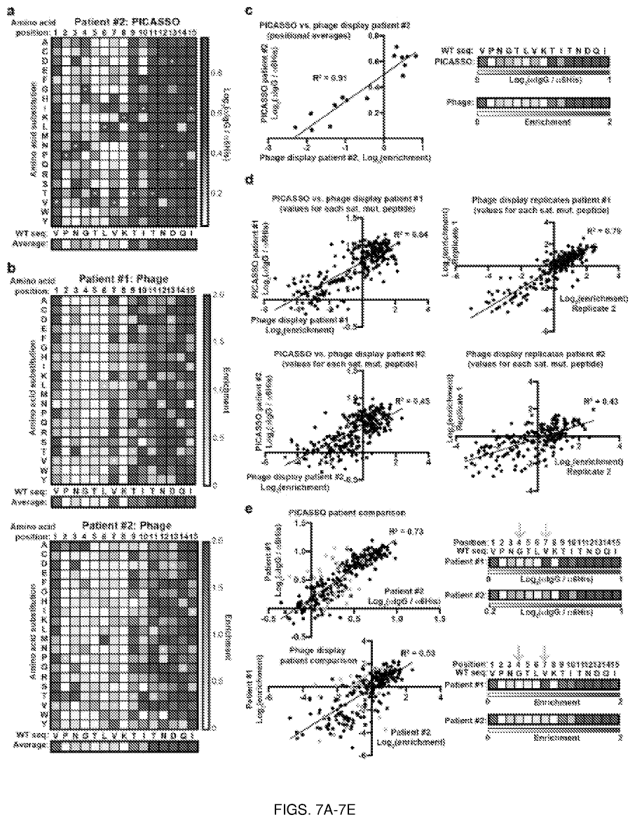

Using the same experimental design and approach as for the FLAG experiments, we created a PICASSO saturation mutagenesis library for an immunodominant epitope from influenza A (IAV) within HA (VPNGTLVKTITNDQI) (e.g., see Xu et al., Science 348, aaa0698 (2015)). The final library encoded 286 variant peptides in quadruplicate paired with unique sgRNAs (1,144 peptide-sgRNA pairs total). We applied serum samples from two patients with known IAV epitope reactivity to these saturation mutagenesis PICASSO microarrays and observed antibody binding profiles (FIG. 3C, FIG. 7A). For both patient samples, the serum antibody binding profiles to the IAV epitope observed by PICASSO correlated with results from patient-matched phage display experiments (R²=0.96 and 0.91 for epitope positional averages, FIG. 3D, FIG. 7B-7C). While the correlation of individual IAV variant peptide scores between PICASSO and phage display for each patient sample was weaker (R²=0.64 and 0.45, respectively), it was comparable to the correlation between the two phage display experimental replicates (R²=0.79 and 0.43, respectively, FIG. 7D). By comparing the PICASSO-based antibody binding profiles between the two patients, we discerned the relative differences in the importance of amino acid identity at each position along with length of the IAV epitope for antibody binding coordination. These results reveal slight deviations in antibody-epitope recognition, particularly at positions 4 and 7 between the two samples (FIG. 7E), suggesting highly similar yet nonidentical paratopes. Together, these results highlight PICASSO's ability to quantitatively evaluate antibody epitope binding from unpurified patient serum samples and perform comparisons between antibodies targeting the same epitope.

### SARS-CoV-2 Epitope Mapping and Multiplexed Antibody Monitoring Using PICASSO

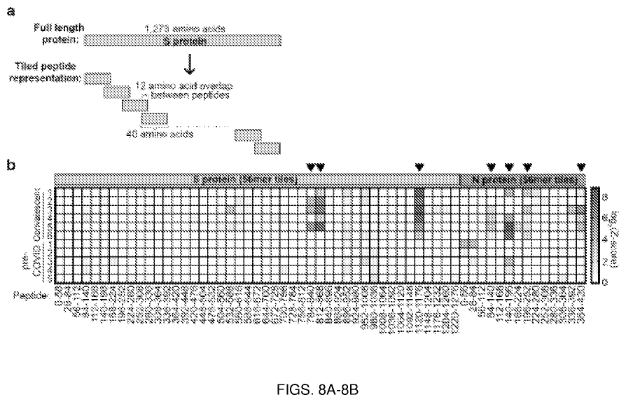

Finally, we evaluated PICASSO's ability to perform mapping experiments to identify linear antibody epitopes within SARS-CoV-2 proteins using COVID-19 convalescent patient sera. By PICASSO, we represented the proteome of SARS-CoV-2 as 40mer peptide tiles with 12 amino acid overlap between adjacent tiles (FIG. 8A), resulting in a 122-member peptide library encoded in quadruplicate paired with unique sgRNAs (488 peptide-sgRNA total pairings). We excluded open reading frame 1ab (ORF1 ab) because of its large size and homology to proteins from related coronaviruses. Four linear epitopes (S protein: 792-832; S protein: 1128-1168; N protein: 148-188; and N protein: 364-420) scored above a PICASSO anti-IgG:total protein signal ratio of 1.3 in three or more of the tested convalescent samples (n=6) and did not appear to be recognized by patient antibodies from pre-pandemic control sera (n=5, FIG. 3E). Concordant with these results, these shared epitopes (or adjacent overlapping peptide tiles) were previously discovered in the same patient samples investigated using a phage display-based platform for SARS-CoV-2 viral epitope identification (VirScan)(e.g., see Shrock et al., Science 370, eabd4250 (2020)) using a Z-score threshold of 3.5 (FIG. 8B). These results suggest that PICASSO can be developed into a diagnostic tool for polyclonal antibody monitoring. Two additional shared peptides in these patient samples were identified by VirScan in the N protein (N: 84-140 and N:196-252) that were not identified by PICASSO.

Taken together, these results demonstrate that PICASSO is an efficient technique to generate complex self-assembling protein microarrays for epitope mapping and quantitative antibody binding characterization applications. Differences in detected antibodies toward peptides derived from SARS-CoV-2 were observed between PICASSO and VirScan. These differences could be due to differential steric presentation or peptide copy number, resulting in reduced antibody capture efficiency or avidity effects. In some embodiments PICASSO's sensitivity and performance is enhanced by altering oligonucleotide spacing density on the microarray surface, optimizing linker length and composition between dCas9 and its fusion partners, improving experimental conditions such as buffer compositions and serum antibody concentrations, and/or processing large patient cohorts for the establishment of rigorous antibody detection thresholds.

Our experiments evaluated PICASSO's compatibility with peptides up to 40 amino acids in length expressed in E. coli. We anticipate that longer, full-length proteins presented by PICASSO will be possible, enabling study of conformational epitopes. Engineered heterologous systems (e.g., see Pirman et al., Nat Commun 6, 8130 (2015); Barber et al., Nat Biotechnol 36, 638-644 (2018); Wachter et al., Adv Biochem Eng Biotechnology 1-43 (2018)) or eukaryotic cells lines may also be employed for dCas9-fusion library expression to represent protein folding and posttranslational modifications in higher organisms.

We have developed and characterized a novel CRISPR-based protein display platform for high-throughput in vitro protein studies. In developing PICASSO, we have performed the first demonstration of multiplexed protein library self-assembly using a CRISPR-based system, making rapid, custom protein studies feasible in any laboratory with access to common molecular biology reagents. While we interfaced these dCas9-fusion libraries with dsDNA microarrays for large-scale protein assays, the PICASSO immobilization strategy could assist in future biomaterials fabrication in which multiple protein species are desired at spatially distinct positions on solid surfaces, requiring only the placement of target dsDNA molecules at defined locations. We anticipate that dCas9-based protein display and PICASSO will be useful for the investigation of customized protein libraries for many additional applications, including multiplexed diagnostics, enzyme substrate discovery, and protein evolution and design experiments.

The above examples, described in Example 1, were prepared using the following materials and methods.

### Materials and Methods

dCas9 & sgRNA Cloning and Plasmid Library Construction

Plasmids encoding anhydrotetracycline-inducible dCas9 (pdCas9-bacteria #44249) and constitutively expressed sgRNA (pgRNA-bacteria #44251) were obtained from Addgene (e.g., see Qi et al., Cell 152, 1173-1183 (2013)). The plasmid for expression of dCas9-6His used for experiments in FIG. 1 and FIG. 4B-4E were generated by the addition of a multiple cloning site consisting of tandem Sall, BsrGI, and PvuI sites followed by a 6His tag immediately 5′ to the TAA stop codon in pdCas9-bacteria.

Expression plasmids for dCas9-epitope fusions in FIG. 2, FIG. 4A and FIG. 5A were created by introducing epitope tag sequences between the BsrGI and PvuI sites in the dCas9-6His expression vector. For sgRNAs used in FIG. 1, FIG. 2, FIG. 4, and FIG. 5A, 20 bp spacer sequences were introduced into pgRNA in between the SpeI restriction site and the sgRNA scaffold. The vector used for dCas9-fusion and paired sgRNA library expression from a single plasmid (“library expression vector”) was generated by insertion of the sgRNA scaffold from pgRNA into the region between the 3′ end of the dCas9 locus and the plasmid origin in pdCas9 (annotated plasmid sequence included below). A C-terminal GGGGS linker was also included on dCas9 in this vector.

230mer oligonucleotide libraries encoding the paired peptide-sgRNA sequences used in FIG. 3, FIG. 5B-5G, FIG. 6, FIG. 7 and FIG. 8 were synthesized by Agilent as a single mixed pool. As depicted in FIG. 5B, each oligonucleotide contained, from 5′ to 3′: 1) a universal primer binding site for oligos related to each independent experiment (F); 2) a subpool primer binding site for oligos containing 1 of the 4 experimental sgRNA replicates (F); 3) a homology region for HiFi assembly (F); 4) the encoded peptide, codon optimized using naturally occurring codon frequencies in K12 E. coli with rare codons eliminated (Xu et al., Science 348, aaa0698 (2015)); 5) a Sall restriction site; 6) randomized bases to bring the total sequence length to 230 bp; 7) an SpeI restriction site; 8) a 20 bp sgRNA spacer; 9) a homology region for HiFi assembly (R); 10) a subpool primer binding site (R); and 11) a universal primer binding site (R). Primer annealing regions and sgRNA spacer sequences were derived as substrings from a previously-developed list of orthogonal 25mer sequences (Xu et al., Proc National Acad Sci 106, 2289-2294 (2009)). 20 bp sgRNA spacers were chosen that were not identical to any other library spacer within the first 6 bp of the seed sequence and that did not contain any extraneous PAMs (‘NGG’) on the sense strand to reduce off-target DNA binding on the corresponding DNA microarray. Spacer sequences were also selected for which the secondary structure of its corresponding ssDNA probe on the oligonucleotide template microarray would not be too high and therefore impede primer annealing for dsDNA microarray generation; target ssDNA sequences for each sgRNA were ensured to have a minimum free energy of −5 kcal/mol or greater by RNAfold 2.4.11 using parameters for ssDNA (Hofacker et al. Nucleic Acids Res 31, 3429-3431 (2003)). Universal and subpool primer binding site lengths varied depending on the encoded peptide length and remaining space on the oligonucleotide. Oligonucleotide library sequences are listed in Table 1.

Oligonucleotide library subpools were PCR amplified using subpool primers complementary to the subpool primer annealing regions with Q5 (NEB) and 10 amplification cycles. PCR products were gel extracted on a 2% agarose gel and then further amplified using primers that annealed within the HiFi assembly homology regions and 5 amplification cycles. The PCR product was then column purified and concentration was measured by NanoDrop A₂₈₀. 100 ng of PvuI/BsaI-digested library expression vector and 10 ng of the insert library were used in a 20 μL HiFi (NEB) assembly reaction at 50° C. for 1 h, desalted using 0.7×AMPure XP beads (Beckman Coulter), and the whole reaction was transformed into 10 μL ElectroMAX DH10B cells (Thermo Fisher). Recovered cells were plated on 15 cm LB agar plates containing 50 μg/mL carbenicillin. After 16 h at 37° C., bacterial libraries were scraped from the plates and miniprepped. The resulting plasmid library (“precursor library”) was then digested with Sall/SpeI, and 100 ng of the library was used for ligation for 16 h at 16° C. with T4 ligase (NEB) with 50 ng of a Sall/SpeI-digested DNA fragment (“expression scaffold”) containing from 5′ to 3′: 1) a Sall site; 2) HA and 6His universal epitope tags for total protein normalization; 3) a TAA stop codon; 4) a camR expression cassette, for chloramphenicol-based selection of plasmids containing this insert; 5) a T7 promoter for inducible sgRNA expression; and 6) an SpeI site. The ligation was then desalted, transformed into a cloning strain, recovered and plated on 15 cm LB agar+50 μg/mL carbenicillin+25 μg/mL chloramphenicol, and purified as above (“final library”). The library insert of the precursor vector library (spanning the encoded peptides and paired sgRNA) was evaluated by limited ˜100,000-read 2×150 bp Illumina-based sequencing (Massachusetts General Hospital Center for Computational Biology DNA Core) to establish library completeness and correct peptide-sgRNA pairings, which were on average both >99% for all generated precursor libraries. For the final vector libraries, only the peptide region was sequenced, using a similar protocol and again showing >99% completeness.

dCas9-Fusion Library Peptide Design

The saturation mutagenesis libraries for FLAG (DYKDDDDK) and the IAV immunodominant peptide (VPNGTLVKTITNDQI) both contained peptide variants with substitution of each amino acid to every other of the 19 possible amino acids. For SARS-CoV-2 epitope mapping experiments, we represented the proteome of SARS-CoV-2 as 40mer peptide tiles with 12 amino acid overlap between adjacent tiles (FIG. 8A), which were centered around the same residues as the 56mer tiles used in the comparison VirScan dataset in FIG. 8B (i.e. both PICASSO and VirScan libraries contained the same number of tiles but with different lengths and tile overlap). Each of these peptides was codon optimized in quadruplicate (i.e. each had a slightly different DNA composition encoding the same amino acids) and each replicate was paired with a unique sgRNA sequence, following the design principles stated above. The paired peptide+sgRNA sequences were then encoded in oligonucleotides as described above, listed in Table 1. 154-member peptide library encoding 12 FLAG peptide with unique sgRNAs and used for the library-scale localization control experiments shown in FIG. 5C-5G encoded primarily a subset of peptides from SARS-CoV-2 and was not used for further experiments in this paper (sequences listed in Table 1).

Oligonucleotide Microarray Design & Conversion to dsDNA Microarrays

Oligonucleotide microarrays were ordered from Customarray (GenScript) containing 4 subarrays each with 2,240 individual features. Each 50mer ssDNA sequence, connected to the microarray surface at its 3′ end, was designed with the following sequence (PAM underlined): 5′-GAGCGACGCTGCACCA-[20 bp corresponding to sgRNA]-CCCGACCTCACCCG-3′. 20 bp target sequences were chosen corresponding to the orthogonalized sgRNA sequences for each PICASSO experiment. Oligonucleotides were printed in duplicate for FIG. 1D-1E and FIG. 4 and for off-target sequences in FIG. 2 and FIG. 5A, and printed in triplicate for all other experiments unless otherwise noted.

To create dsDNA microarrays, oligonucleotide microarrays fitted with a hybridization cap (CustomArray) with 30 μL capacity for each subarray were treated with 30 μL water and incubated at 70° C. for 10 min. In a 50 mL Falcon tube, microarrays were then treated with 40 mL 1 M NaOH for 5 min, repeated once, and then rinsed in PBS. Subarrays were then rinsed twice using the hybridization cap with 30 μL 1× Thermopol buffer (NEB). The following was then added to each subarray: 3 μL 10× Thermopol buffer (NEB), 0.6 μL 1 mM dNTPs (NEB), 0.6 μL 0.1 mM Cy3-dUTP (Millipore Sigma), 15 μL 10 μM extension primer (5′-AC+G+G+GT+GAGGTCGGG-3′, where + denotes LNA bases, synthesized by IDT), 0.6 μL Vent Exo-(NEB), and 10.2 μL water. The microarrays were then placed in an oven with rotisserie-style mixing, subjected to the following heat cycle: 10 min intervals in 5° C. increments from at 85° C. to 55° C., then the following repeated twice: 15 min at 65° C., 15 min at 72° C., 15 min at 65° C., 15 min at 55° C. Microarrays were then held at 55° C. for 4 h and then stored at 4° C. for 16 h.

dCas9-Fusion Library Expression and Purification for PICASSO

Two-plasmid expression of dCas9-fusion and sgRNA was performed by double plasmid transformation into BL21(DE3) electrocompetent cells (Sigma-Aldrich) as in FIG. 1, FIG. 2, FIG. 4 and FIG. 5A. For single-backbone libraries encoding peptide/sgRNA pairs on the same plasmid, 1 μL of ˜300 ng/μL final plasmid libraries were transformed into T7 Express lys Y Competent E. coli (High Efficiency, NEB), with each library subpool transformed separately. Transformed cells were recovered for 1 h at 37° C. in 1 mL SOC media (NEB). Recovered cells were inoculated directly into 50 mL LB+50 μg/mL carbenicillin+25 μg/mL chloramphenicol and incubated at 37° C. for 16 hours, shaking at 230 rpm. Cells were then diluted to OD₆₀₀=0.2 in 250 mL LB+50 μg/mL carbenicillin+25 μg/mL chloramphenicol and grown in a baffled 1 L flask at 37° C. until they reached OD₆₀₀=0.8. Protein expression was then induced with 100 ng/mL anhydrotetracycline. In lys Y cells only, sgRNA expression was induced with 0.1 mM IPTG. Cells were grown for an additional 4 h at 37° C. Cells were mixed with an equal volume of BL21 (DE3) cells expressing a single control sgRNA and no dCas9 (5′-AUAAAAGAAACCCUCCGCAU-3′ sgRNA spacer sequence in pgRNA) to soak up any dCas9 molecules during protein purification not already associated with an sgRNA, and then harvested by centrifugation at 5,000×g for 10 min. Cell pellets were frozen at −80° C. for at least 16 h.

Cell pellets were lysed by thawing at 37° C. until pellets were runny and then resuspending each pellet in 12.5 mL lysis buffer containing 50 mM Tris pH 7.4, 500 mM NaCl, 10% glycerol, 100 μM DTT, 1 μL rLysozyme solution (Millipore Sigma), 5 μL benzonase (90% purity, Millipore Sigma), 1× BugBuster (Millipore Sigma), and 1× protease inhibitors (cOmplete EDTA-free, Millipore Sigma), mixing at 25° C. for 20 min. Samples were spun down at 5,000×g for 20 min, and lysates transferred to 250 μL bed volume Ni-NTA agarose (Qiagen). Lysates were incubated with resin for 20 min at 25° C., then washed twice with 5 mL wash buffer (50 mM Tris pH 7.4, 500 mM NaCl, 10% glycerol, 100 μM DTT, 20 mM imidazole), and then eluted with 2×500 μL elution buffer (50 mM Tris pH 7.4, 500 mM NaCl, 10% glycerol, 100 μM DTT, 500 mM imidazole). Eluates were passed through a 45 μm filter to remove traces of Ni-NTA resin, and added to an Amicon Ultra-4 centrifugal filter with 100 kDa molecular weight cutoff (Millipore Sigma). Samples were spun at 4,000×g for 20 min and buffer exchanged with 4 mL storage buffer (50 mM Tris pH 7.4, 150 mM NaCl, 10% glycerol, 1 mM DTT). This was repeated 3 times, with a final purified dCas9-fusion library volume of 50-100 μL. Protein concentration was estimated by A2₆₀ using a NanoDrop, and protein libraries were then applied to dsDNA microarrays or stored at −20° C.

dCas9-Fusion Library Application to Microarrays & Antibody Binding

dsDNA microarrays were blocked with 2% milk in PBST for 30 min at 25° C. Approximately 5 μg of purified individual or dCas9-fusion libraries were added to each dsDNA subarray in storage buffer with 0.05% Tween-20 and 250 μg/mL salmon sperm DNA. For experiments using sublibraries corresponding to quadruplicate peptide replicates with unique sgRNAs, dCas9-fusion library subpools were combined in this step in addition to 1 μg of separately purified dCas9-6His with control sgRNA (spacer: 5′-CCGUACCUAGAUACACUCAA-3′). dCas9-fusion library self-assembly on the dsDNA microarray surface was allowed to occur at 37° C. for 16 h. Subarrays were then washed twice with 30 μL PBST and blocked again with 2% milk in PBST for 30 min at 25° C. Arrays were then treated with the corresponding test antibody using the following dilutions in 2% milk in PBST with 250 μg/mL salmon sperm DNA (ThermoFisher) for 1 h at 25° C.:1:1000 anti-6His (Cell Signaling D3I1O, rabbit), 1:250 anti-HA (Cell Signaling C29F4, rabbit), 1:500 anti-myc (Abcam ab9106, rabbit), 1:250 anti-FLAG M2 (Millipore Sigma F1804, mouse, used in FIG. 3B and FIG. 6), 1:1000 anti-FLAG (Abcam ab205606, rabbit, used in FIG. 2 and FIG. 5A), or 1:10 patient serum (pre-blocked with 250 μg/mL salmon sperm DNA and 100 ng/μL dCas9-6His with control sgRNA). COVID-19 convalescent samples, healthy controls, and samples from patients with previous influenza A exposure were from previous studies^{2,5}. Microarrays employing total protein normalization were then treated for 1 h at 25° C. with 1:1000 anti-6His (rabbit) or 1:250 anti-HA antibody (rabbit), depending on the universal epitope tag indicated for each library preparation. Subarrays were then treated with corresponding secondary fluorescent antibodies for 30 min at 25° C.: 1:40 donkey anti-rabbit IgG H&L-Alexa 647 (Abcam ab150075) for experiments shown in FIG. 1, FIG. 2, FIG. 4, and FIG. 5A); 1:40 goat anti-rabbit IgG H&L-Alexa 488 (Abcam ab150077) for experiments using total protein normalization; 1:40 goat anti-mouse IgG H&L-Alexa 647 (Abcam ab150115) for M2 anti-FLAG experiments (including FIG. 5C-5G); 1:40 goat anti-human IgG (H+L)-Alexa 647 (ThermoFisher A-21445) for experiments involving human serum. Between all antibody applications, subarrays were washed vigorously with PBST. Immediately prior to imaging, microarrays were washed with PBST and then PBS, and topped with a raised coverslip.

### Microarray Scanning & Data Analysis

Microarrays were imaged using a Genepix 4300A microarray scanner (Molecular Devices) at 5 μm resolution using 488 nm, 532 nm, and 635 nm lasers with 70% power and 450 PMT gain (decreased to as low as 40% power and 300 PMT if any features were saturated). Median fluorescence intensity values for each feature using local background subtraction were extracted using GenePix Pro 7 software. Fluorescence values or log ₂ transformed fluorescence ratios were averaged across replicate dsDNA features. Average values <0 were considered to be below the limit of detection. For the FLAG and influenza A epitope saturation mutagenesis experiments, values for the quadruplicate variant peptides with unique sgRNAs were averaged for analysis. For SARS-CoV-2 libraries, due to variable background and technical faults (i.e. fluorescent splotches that occur irregularly outside of the dsDNA features), any dsDNA sequence for which 2*(minimum technical replicate value)>(maximum technical replicate value) was eliminated and only the highest fluorescence ratio value for the sgRNA replicates was used for analysis. Additional background subtraction was performed in FIG. 1 and FIG. 4 by subtracting the median fluorescence value of a dsDNA control with perfect match to the corresponding sgRNA and no PAM (5′-CCA-3′ changed to 5′-CGA-3′ in synthesized microarray oligonucleotide). Heat maps were generated in Prism 9. Maximum color intensities in the heat maps for the saturation mutagenesis libraries was set to the average wildtype peptide sequence. The sequence logo in FIG. 6C was made using Logomaker (Tareen et al., Bioinformatics 36, 2272-2274 (2019)). The top 50% of normalized non-log ₂-transformed anti-FLAG signals from FIG. 3B were scaled to create mock frequencies at each position along the FLAG epitope, which were then converted to represent information content and relative amino acid frequency at each position as previously described (Schneider et al., Nucleic Acids Res 18, 6097-6100 (1990)).

### Phage Immunoprecipitation and Sequencing

We designed a saturation mutagenesis library for the IAV immunodominant epitope VPNGTLVKTITNDQI, substituting each amino acid to each of the 19 other possible natural amino acids, and created a phage library using previously described library design and production protocols (e.g., see Xu et al., Science 348, aaa0698 (2015)). We performed phage immunoprecipitation and sequencing as described previously with slight modifications (e.g., see Xu et al., Science 348, aaa0698 (2015)). For the immunoprecipitation, we added 5 mg biotinylated Goat Anti-Human Kappa (Southern Biotech) antibodies to the phage and serum mixture and incubated the reactions at 4° C. overnight. Then, we added 20 μL of Pierce Streptavidin Magnetic Beads (Thermo), incubated the reactions at room temperature for 4 h and continued with the washes and the remainder of the protocol as previously described (e.g., see Xu et al., Science 348, aaa0698 (2015)).

### Statistical Analysis of Phage Display Data

We mapped the sequencing reads to the reference library using Bowtie (e.g., see Langmead et al., Genome Biol 10, R25 (2009)). For each sample, we divided the number of reads corresponding to each peptide clone by the total number of reads for the sample to obtain the fractional abundance of each peptide clone. Then, we divided the fractional abundance of each peptide clone in the sample by that in the input library to obtain the enrichment value.

### Exemplary Oligonucleotide Library Sequences

As described herein, a “library expression vector” may be used for single plasmid-based co-expression of paired dCas9-fusion and sgRNA. As described herein, exemplary nucleic acid sequences that may be included in a library expression vector are exemplified by the nucleic acid sequences in Table 1, shown below.

TABLE 1
Exemplary Nucleic Acid Sequences of a Library Expression Vector

| SEQ ID NO | Description | Sequence |
|---|---|---|
| 1 | Tetracycline repressor | TTAAGACCCACTTTCACATTTAAGTTGTTTTTCTAATC |
| | (e.g., repressor of the | CGCATATGATCAATTCAAGGCCGAATAAGAAGGCTGG |
| | tetracycline resistance | CTCTGCACCTTGGTGATCAAATAATTCGATAGCTTGT |
| | element) | CGTAATAATGGCGGCATACTATCAGTAGTAGGTGTTT |
| | | CCCTTTCTTCTTTAGCGACTTGATGCTCTTGATCTTCC |
| | | AATACGCAACCTAAAGTAAAATGCCCCACAGCGCTGA |
| | | GTGCATATAATGCATTCTCTAGTGAAAAACCTTGTTGG |
| | | CATAAAAAGGCTAATTGATTTTCGAGAGTTTCATACTG |
| | | TTTTTCTGTAGGCCGTGTACCTAAATGTACTTTTGCTC |
| | | CATCGCGATGACTTAGTAAAGCACATCTAAAACTTTTA |
| | | GCGTTATTACGTAAAAAATCTTGCCAGCTTTCCCCTTC |
| | | TAAAGGGCAAAAGTGAGTATGGTGCCTATCTAACATC |
| | | TCAATGGCTAAGGCGTCGAGCAAAGCCCGCTTATTTT |
| | | TTACATGCCAATACAATGTAGGCTGCTCTACACCTAG |
| | | CTTCTGGGCGAGTTTACGGGTTGTTAAACCTTCGATT |
| | | CCGACCTCATTAAGCAGCTCTAATGCGCTGTTAATCA |
| | | CTTTACTTTTATCTAATCTAGACAT |
| 2 | dCas9 | ATGGATAAGAAATACTCAATAGGCTTAGCTATCGGCA |
| | | CAAATAGCGTCGGATGGGCGGTGATCACTGATGAAT |
| | | ATAAGGTTCCGTCTAAAAAGTTCAAGGTTCTGGGAAA |
| | | TACAGACCGCCACAGTATCAAAAAAAATCTTATAGGG |
| | | GCTCTTTTATTTGACAGTGGAGAGACAGCGGAAGCGA |
| | | CTCGTCTCAAACGGACAGCTCGTAGAAGGTATACACG |
| | | TCGGAAGAATCGTATTTGTTATCTACAGGAGATTTTTT |
| | | CAAATGAGATGGCGAAAGTAGATGATAGTTTCTTTCA |
| | | TCGACTTGAAGAGTCTTTTTTGGTGGAAGAAGACAAG |
| | | AAGCATGAACGTCATCCTATTTTTGGAAATATAGTAGA |
| | | TGAAGTTGCTTATCATGAGAAATATCCAACTATCTATC |
| | | ATCTGCGAAAAAAATTGGTAGATTCTACTGATAAAGC |
| | | GGATTTGCGCTTAATCTATTTGGCCTTAGCGCATATG |
| | | ATTAAGTTTCGTGGTCATTTTTTGATTGAGGGAGATTT |
| | | AAATCCTGATAATAGTGATGTGGACAAACTATTTATCC |
| | | AGTTGGTACAAACCTACAATCAATTATTTGAAGAAAAC |
| | | CCTATTAACGCAAGTGGAGTAGATGCTAAAGCGATTC |
| | | TTTCTGCACGATTGAGTAAATCAAGACGATTAGAAAAT |
| | | CTCATTGCTCAGCTCCCCGGTGAGAAGAAAAATGGCT |
| | | TATTTGGGAATCTCATTGCTTTGTCATTGGGTTTGACC |
| | | CCTAATTTTAAATCAAATTTTGATTTGGCAGAAGATGC |
| | | TAAATTACAGCTTTCAAAAGATACTTACGATGATGATT |
| | | TAGATAATTTATTGGCGCAAATTGGAGATCAATATGCT |
| | | GATTTGTTTTTGGCAGCTAAGAATTTATCAGATGCTAT |
| | | TTTACTTTCAGATATCCTAAGAGTAAATACTGAAATAA |
| | | CTAAGGCTCCCCTATCAGCTTCAATGATTAAACGCTA |
| | | CGATGAACATCATCAAGACTTGACTCTTTTAAAAGCTT |
| | | TAGTTCGACAACAACTTCCAGAAAAGTATAAAGAAAT |
| | | CTTTTTTGATCAATCAAAAAACGGATATGCAGGTTATA |
| | | TTGATGGGGGAGCTAGCCAAGAAGAATTTTATAAATT |
| | | TATCAAACCAATTTTAGAAAAAATGGATGGTACTGAG |
| | | GAATTATTGGTGAAACTAAATCGTGAAGATTTGCTGC |
| | | GCAAGCAACGGACCTTTGACAACGGCTCTATTCCCCA |
| | | TCAAATTCACTTGGGTGAGCTGCATGCTATTTTGAGA |
| | | AGACAAGAAGACTTTTATCCATTTTTAAAAGACAATCG |
| | | TGAGAAGATTGAAAAAATCTTGACTTTTCGAATTCCTT |
| | | ATTATGTTGGTCCATTGGCGCGTGGCAATAGTCGTTT |
| | | TGCATGGATGACTCGGAAGTCTGAAGAAACAATTACC |
| | | CCATGGAATTTTGAAGAAGTTGTCGATAAAGGTGCTT |
| | | CAGCTCAATCATTTATTGAACGCATGACAAACTTTGAT |
| | | AAAAATCTTCCAAATGAAAAAGTACTACCAAAACATAG |
| | | TTTGCTTTATGAGTATTTTACGGTTTATAACGAATTGA |
| | | CAAAGGTCAAATATGTTACTGAAGGAATGCGAAAACC |
| | | AGCATTTCTTTCAGGTGAACAGAAGAAAGCCATTGTT |
| | | GATTTACTCTTCAAAACAAATCGAAAAGTAACCGTTAA |
| | | GCAATTAAAAGAAGATTATTTCAAAAAAATAGAATGTT |
| | | TTGATAGTGTTGAAATTTCAGGAGTTGAAGATAGATTT |
| | | AATGCTTCATTAGGTACCTACCATGATTTGCTAAAAAT |
| | | TATTAAAGATAAAGATTTTTTGGATAATGAAGAAAATG |
| | | AAGATATCTTAGAGGATATTGTTTTAACATTGACCTTA |
| | | TTTGAAGATAGGGAGATGATTGAGGAAAGACTTAAAA |
| | | CATATGCTCACCTCTTTGATGATAAGGTGATGAAACA |
| | | GCTTAAACGTCGCCGTTATACTGGTTGGGGACGTTTG |
| | | TCTCGAAAATTGATTAATGGTATTAGGGATAAGCAATC |
| | | TGGCAAAACAATATTAGATTTTTTGAAATCAGATGGTT |
| | | TTGCCAATCGCAATTTTATGCAGCTGATCCATGATGAT |
| | | AGTTTGACATTTAAAGAAGACATTCAAAAAGCACAAGT |
| | | GTCTGGACAAGGCGATAGTTTACATGAACATATTGCA |
| | | AATTTAGCTGGTAGCCCTGCTATTAAAAAAGGTATTTT |
| | | ACAGACTGTAAAAGTTGTTGATGAATTGGTCAAAGTA |
| | | ATGGGGCGGCATAAGCCAGAAAATATCGTTATTGAAA |
| | | TGGCACGTGAAAATCAGACAACTCAAAAGGGCCAGA |
| | | AAAATTCGCGAGAGCGTATGAAACGAATCGAAGAAG |
| | | GTATCAAAGAATTAGGAAGTCAGATTCTTAAAGAGCA |
| | | TCCTGTTGAAAATACTCAATTGCAAAATGAAAAGCTCT |
| | | ATCTCTATTATCTCCAAAATGGAAGAGACATGTATGTG |
| | | GACCAAGAATTAGATATTAATCGTTTAAGTGATTATGA |
| | | TGTCGATGCCATTGTTCCACAAAGTTTCCTTAAAGAC |
| | | GATTCAATAGACAATAAGGTCTTAACGCGTTCTGATAA |
| | | AAATCGTGGTAAATCGGATAACGTTCCAAGTGAAGAA |
| | | GTAGTCAAAAAGATGAAAAACTATTGGAGACAACTTC |
| | | TAAACGCCAAGTTAATCACTCAACGTAAGTTTGATAAT |
| | | TTAACGAAAGCTGAACGTGGAGGTTTGAGTGAACTTG |
| | | ATAAAGCTGGTTTTATCAAACGCCAATTGGTTGAAACT |
| | | CGCCAAATCACTAAGCATGTGGCACAAATTTTGGATA |
| | | GTCGCATGAATACTAAATACGATGAAAATGATAAACTT |
| | | ATTCGAGAGGTTAAAGTGATTACCTTAAAATCTAAATT |
| | | AGTTTCTGACTTCCGAAAAGATTTCCAATTCTATAAAG |
| | | TACGTGAGATTAACAATTACCATCATGCCCATGATGC |
| | | GTATCTAAATGCCGTCGTTGGAACTGCTTTGATTAAG |
| | | AAATATCCAAAACTTGAATCGGAGTTTGTCTATGGTGA |
| | | TTATAAAGTTTATGATGTTCGTAAAATGATTGCTAAGT |
| | | CTGAGCAAGAAATAGGCAAAGCAACCGCAAAATATTT |
| | | CTTTTACTCTAATATCATGAACTTCTTCAAAACAGAAA |
| | | TTACACTTGCAAATGGAGAGATTCGCAAACGCCCTCT |
| | | AATCGAAACTAATGGGGAAACTGGAGAAATTGTCTGG |
| | | GATAAAGGGCGAGATTTTGCCACAGTGCGCAAAGTAT |
| | | TGTCCATGCCCCAAGTCAATATTGTCAAGAAAACAGA |
| | | AGTACAGACAGGCGGATTCTCCAAGGAGTCAATTTTA |
| | | CCAAAAAGAAATTCGGACAAGCTTATTGCTCGTAAAA |
| | | AAGACTGGGATCCAAAAAAATATGGTGGTTTTGATAG |
| | | TCCAACGGTAGCTTATTCAGTCCTAGTGGTTGCTAAG |
| | | GTGGAAAAAGGGAAATCGAAGAAGTTAAAATCCGTTA |
| | | AAGAGTTACTAGGGATCACAATTATGGAAAGAAGTTC |
| | | CTTTGAAAAAAATCCGATTGACTTTTTAGAAGCTAAAG |
| | | GATATAAGGAAGTTAAAAAAGACTTAATCATTAAACTA |
| | | CCTAAATATAGTCTTTTTGAGTTAGAAAACGGTCGTAA |
| | | ACGGATGCTGGCTAGTGCCGGAGAATTACAAAAAGG |
| | | AAATGAGCTGGCTCTGCCAAGCAAATATGTGAATTTT |
| | | TTATATTTAGCTAGTCATTATGAAAAGTTGAAGGGTAG |
| | | TCCAGAAGATAACGAACAAAAACAATTGTTTGTGGAG |
| | | CAGCATAAGCATTATTTAGATGAGATTATTGAGCAAAT |
| | | CAGTGAATTTTCTAAGCGTGTTATTTTAGCAGATGCCA |
| | | ATTTAGATAAAGTTCTTAGTGCATATAACAAACATAGA |
| | | GACAAACCAATACGTGAACAAGCAGAAAATATTATTC |
| | | ATTTATTTACGTTGACGAATCTTGGAGCTCCCGCTGC |
| | | TTTTAAATATTTTGATACAACAATTGATCGTAAACGATA |
| | | TACGTCTACAAAAGAAGTTTTAGATGCCACTCTTATCC |
| | | ATCAATCCATCACTGGTCTTTATGAAACACGCATTGAT |
| | | TTGAGTCAGCTAGGAGGTGAC |
| 3 | GGGGS linker (e.g., C- | GGTGGAGGAGGTTCT |
| | terminal linker) | |
| 4 | dCas9 with C-terminal | ATGGATAAGAAATACTCAATAGGCTTAGCTATCGGCA |
| | GGGGS linker | CAAATAGCGTCGGATGGGGGGTGATCACTGATGAAT |
| | | ATAAGGTTCCGTCTAAAAAGTTCAAGGTTCTGGGAAA |
| | | TACAGACCGCCACAGTATCAAAAAAAATCTTATAGGG |
| | | GCTCTTTTATTTGACAGTGGAGAGACAGCGGAAGCGA |
| | | CTCGTCTCAAACGGACAGCTCGTAGAAGGTATACACG |
| | | TCGGAAGAATCGTATTTGTTATCTACAGGAGATTTTTT |
| | | CAAATGAGATGGCGAAAGTAGATGATAGTTTCTTTCA |
| | | TCGACTTGAAGAGTCTTTTTTGGTGGAAGAAGACAAG |
| | | AAGCATGAACGTCATCCTATTTTTGGAAATATAGTAGA |
| | | TGAAGTTGCTTATCATGAGAAATATCCAACTATCTATC |
| | | ATCTGCGAAAAAAATTGGTAGATTCTACTGATAAAGC |
| | | GGATTTGCGCTTAATCTATTTGGCCTTAGCGCATATG |
| | | ATTAAGTTTCGTGGTCATTTTTTGATTGAGGGAGATTT |
| | | AAATCCTGATAATAGTGATGTGGACAAACTATTTATCC |
| | | AGTTGGTACAAACCTACAATCAATTATTTGAAGAAAAC |
| | | CCTATTAACGCAAGTGGAGTAGATGCTAAAGCGATTC |
| | | TTTCTGCACGATTGAGTAAATCAAGACGATTAGAAAAT |
| | | CTCATTGCTCAGCTCCCCGGTGAGAAGAAAAATGGCT |
| | | TATTTGGGAATCTCATTGCTTTGTCATTGGGTTTGACC |
| | | CCTAATTTTAAATCAAATTTTGATTTGGCAGAAGATGC |
| | | TAAATTACAGCTTTCAAAAGATACTTACGATGATGATT |
| | | TAGATAATTTATTGGCGCAAATTGGAGATCAATATGCT |
| | | GATTTGTTTTTGGCAGCTAAGAATTTATCAGATGCTAT |
| | | TTTACTTTCAGATATCCTAAGAGTAAATACTGAAATAA |
| | | CTAAGGCTCCCCTATCAGCTTCAATGATTAAACGCTA |
| | | CGATGAACATCATCAAGACTTGACTCTTTTAAAAGCTT |
| | | TAGTTCGACAACAACTTCCAGAAAAGTATAAAGAAAT |
| | | CTTTTTTGATCAATCAAAAAACGGATATGCAGGTTATA |
| | | TTGATGGGGGAGCTAGCCAAGAAGAATTTTATAAATT |
| | | TATCAAACCAATTTTAGAAAAAATGGATGGTACTGAG |
| | | GAATTATTGGTGAAACTAAATCGTGAAGATTTGCTGC |
| | | GCAAGCAACGGACCTTTGACAACGGCTCTATTCCCCA |
| | | TCAAATTCACTTGGGTGAGCTGCATGCTATTTTGAGA |
| | | AGACAAGAAGACTTTTATCCATTTTTAAAAGACAATCG |
| | | TGAGAAGATTGAAAAAATCTTGACTTTTCGAATTCCTT |
| | | ATTATGTTGGTCCATTGGCGCGTGGCAATAGTCGTTT |
| | | TGCATGGATGACTCGGAAGTCTGAAGAAACAATTACC |
| | | CCATGGAATTTTGAAGAAGTTGTCGATAAAGGTGCTT |
| | | CAGCTCAATCATTTATTGAACGCATGACAAACTTTGAT |
| | | AAAAATCTTCCAAATGAAAAAGTACTACCAAAACATAG |
| | | TTTGCTTTATGAGTATTTTACGGTTTATAACGAATTGA |
| | | CAAAGGTCAAATATGTTACTGAAGGAATGCGAAAACC |
| | | AGCATTTCTTTCAGGTGAACAGAAGAAAGCCATTGTT |
| | | GATTTACTCTTCAAAACAAATCGAAAAGTAACCGTTAA |
| | | GCAATTAAAAGAAGATTATTTCAAAAAAATAGAATGTT |
| | | TTGATAGTGTTGAAATTTCAGGAGTTGAAGATAGATTT |
| | | AATGCTTCATTAGGTACCTACCATGATTTGCTAAAAAT |
| | | TATTAAAGATAAAGATTTTTTGGATAATGAAGAAAATG |
| | | AAGATATCTTAGAGGATATTGTTTTAACATTGACCTTA |
| | | TTTGAAGATAGGGAGATGATTGAGGAAAGACTTAAAA |
| | | CATATGCTCACCTCTTTGATGATAAGGTGATGAAACA |
| | | GCTTAAACGTCGCCGTTATACTGGTTGGGGACGTTTG |
| | | TCTCGAAAATTGATTAATGGTATTAGGGATAAGCAATC |
| | | TGGCAAAACAATATTAGATTTTTTGAAATCAGATGGTT |
| | | TTGCCAATCGCAATTTTATGCAGCTGATCCATGATGAT |
| | | AGTTTGACATTTAAAGAAGACATTCAAAAAGCACAAGT |
| | | GTCTGGACAAGGCGATAGTTTACATGAACATATTGCA |
| | | AATTTAGCTGGTAGCCCTGCTATTAAAAAAGGTATTTT |
| | | ACAGACTGTAAAAGTTGTTGATGAATTGGTCAAAGTA |
| | | ATGGGGCGGCATAAGCCAGAAAATATCGTTATTGAAA |
| | | TGGCACGTGAAAATCAGACAACTCAAAAGGGCCAGA |
| | | AAAATTCGCGAGAGCGTATGAAACGAATCGAAGAAG |
| | | GTATCAAAGAATTAGGAAGTCAGATTCTTAAAGAGCA |
| | | TCCTGTTGAAAATACTCAATTGCAAAATGAAAAGCTCT |
| | | ATCTCTATTATCTCCAAAATGGAAGAGACATGTATGTG |
| | | GACCAAGAATTAGATATTAATCGTTTAAGTGATTATGA |
| | | TGTCGATGCCATTGTTCCACAAAGTTTCCTTAAAGAC |
| | | GATTCAATAGACAATAAGGTCTTAACGCGTTCTGATAA |
| | | AAATCGTGGTAAATCGGATAACGTTCCAAGTGAAGAA |
| | | GTAGTCAAAAAGATGAAAAACTATTGGAGACAACTTC |
| | | TAAACGCCAAGTTAATCACTCAACGTAAGTTTGATAAT |
| | | TTAACGAAAGCTGAACGTGGAGGTTTGAGTGAACTTG |
| | | ATAAAGCTGGTTTTATCAAACGCCAATTGGTTGAAACT |
| | | CGCCAAATCACTAAGCATGTGGCACAAATTTTGGATA |
| | | GTCGCATGAATACTAAATACGATGAAAATGATAAACTT |
| | | ATTCGAGAGGTTAAAGTGATTACCTTAAAATCTAAATT |
| | | AGTTTCTGACTTCCGAAAAGATTTCCAATTCTATAAAG |
| | | TACGTGAGATTAACAATTACCATCATGCCCATGATGC |
| | | GTATCTAAATGCCGTCGTTGGAACTGCTTTGATTAAG |
| | | AAATATCCAAAACTTGAATCGGAGTTTGTCTATGGTGA |
| | | TTATAAAGTTTATGATGTTCGTAAAATGATTGCTAAGT |
| | | CTGAGCAAGAAATAGGCAAAGCAACCGCAAAATATTT |
| | | CTTTTACTCTAATATCATGAACTTCTTCAAAACAGAAA |
| | | TTACACTTGCAAATGGAGAGATTCGCAAACGCCCTCT |
| | | AATCGAAACTAATGGGGAAACTGGAGAAATTGTCTGG |
| | | GATAAAGGGCGAGATTTTGCCACAGTGCGCAAAGTAT |
| | | TGTCCATGCCCCAAGTCAATATTGTCAAGAAAACAGA |
| | | AGTACAGACAGGCGGATTCTCCAAGGAGTCAATTTTA |
| | | CCAAAAAGAAATTCGGACAAGCTTATTGCTCGTAAAA |
| | | AAGACTGGGATCCAAAAAAATATGGTGGTTTTGATAG |
| | | TCCAACGGTAGCTTATTCAGTCCTAGTGGTTGCTAAG |
| | | GTGGAAAAAGGGAAATCGAAGAAGTTAAAATCCGTTA |
| | | AAGAGTTACTAGGGATCACAATTATGGAAAGAAGTTC |
| | | CTTTGAAAAAAATCCGATTGACTTTTTAGAAGCTAAAG |
| | | GATATAAGGAAGTTAAAAAAGACTTAATCATTAAACTA |
| | | CCTAAATATAGTCTTTTTGAGTTAGAAAACGGTCGTAA |
| | | ACGGATGCTGGCTAGTGCCGGAGAATTACAAAAAGG |
| | | AAATGAGCTGGCTCTGCCAAGCAAATATGTGAATTTT |
| | | TTATATTTAGCTAGTCATTATGAAAAGTTGAAGGGTAG |
| | | TCCAGAAGATAACGAACAAAAACAATTGTTTGTGGAG |
| | | CAGCATAAGCATTATTTAGATGAGATTATTGAGCAAAT |
| | | CAGTGAATTTTCTAAGCGTGTTATTTTAGCAGATGCCA |
| | | ATTTAGATAAAGTTCTTAGTGCATATAACAAACATAGA |
| | | GACAAACCAATACGTGAACAAGCAGAAAATATTATTC |
| | | ATTTATTTACGTTGACGAATCTTGGAGCTCCCGCTGC |
| | | TTTTAAATATTTTGATACAACAATTGATCGTAAACGATA |
| | | TACGTCTACAAAAGAAGTTTTAGATGCCACTCTTATCC |
| | | ATCAATCCATCACTGGTCTTTATGAAACACGCATTGAT |
| | | TTGAGTCAGCTAGGAGGTGACGGTGGAGGAGGTTCT |
| 5 | PvuI restriction | CGATCG |
| | endonuclease restriction | |
| | site | |
| 6 | Red fluorescent protein | ATGGCGAGTAGCGAAGACGTTATCAAAGAGTTCATGC |
| | | GTTTCAAAGTTCGTATGGAAGGTTCCGTTAACGGTCA |
| | | CGAGTTCGAAATCGAAGGTGAAGGTGAAGGTCGTCC |
| | | GTACGAAGGTACCCAGACCGCTAAACTGAAAGTTACC |
| | | AAAGGTGGTCCGCTGCCGTTCGCTTGGGACATCCTG |
| | | TCCCCGCAGTTCCAGTACGGTTCCAAAGCTTACGTTA |
| | | AACACCCGGCTGACATCCCGGACTACCTGAAACTGT |
| | | CCTTCCCGGAAGGTTTCAAATGGGAACGTGTTATGAA |
| | | CTTCGAAGACGGTGGTGTTGTTACCGTTACCCAGGAC |
| | | TCCTCCCTGCAAGACGGTGAGTTCATCTACAAAGTTA |
| | | AACTGCGTGGTACCAACTTCCCGTCCGACGGTCCGG |
| | | TTATGCAGAAAAAAACCATGGGTTGGGAAGCTTCCAC |
| | | CGAACGTATGTACCCGGAAGACGGTGCTCTGAAAGG |
| | | TGAAATCAAAATGCGTCTGAAACTGAAAGACGGTGGT |
| | | CACTACGACGCTGAAGTTAAAACCACCTACATGGCTA |
| | | AAAAACCGGTTCAGCTGCCGGGTGCTTACAAAACCG |
| | | ACATCAAACTGGACATCACCTCCCACAACGAAGACTA |
| | | CACCATCGTTGAACAGTACGAACGTGCTGAAGGTCGT |
| | | CACTCCACCGGTGCTTAA |
| 7 | BsaI restriction | GGTCTC |
| | endonuclease restriction | |
| | site | |
| 8 | sgRNA scaffold | GTTTTAGAGCTAGAAATAGCAAGTTAAAATAAGGCTA |
| | | GTCCGTTATCAACTTGAAAAAGTGGCACCGAGTCGGT |
| | | GC |
| 9 | p15a origin of replication | TTTCCACAGGCTCCGCCCCCCTGACGAGCATCACAA |
| | site | AAATCGACGCTCAAGTCAGAGGTGGCGAAACCCGAC |
| | | AGGACTATAAAGATACCAGGCGTTTCCCCCTGGAAGC |
| | | TCCCTCGTGCGCTCTCCTGTTCCGACCCTGCCGCTTA |
| | | CCGGATACCTGTCCGCCTTTCTCCCTTCGGGAAGCG |
| | | TGGCGCTTTCTCATAGCTCACGCTGTAGGTATCTCAG |
| | | TTCGGTGTAGGTCGTTCGCTCCAAGCTGGGCTGTGT |
| | | GCACGAACCCCCCGTTCAGCCCGACCGCTGCGCCTT |
| | | ATCCGGTAACTATCGTCTTGAGTCCAACCCGGTAAGA |
| | | CACGACTTATCGCCACTGGCAGCAGCCACTGGTAAC |
| | | AGGATTAGCAGAGCGAGGTATGTAGGCGGTGCTACA |
| | | GAGTTCTTGAAGTGGTGGCCTAACTACGGCTACACTA |
| | | GAAGAACAGTATTTGGTATCTGCGCTCTGCTGAAGCC |
| | | AGTTACCTTCGGAAAAAGAGTTGGTAGCTCTTGATCC |
| | | GGCAAACAAACCACCGCTGGTAGCGGTGGTTTTTTTG |
| | | TTTGCAAGCAGCAGATTACGCGCAGAAAAAAAGGATC |
| | | TCAA |
| 10 | ampR (e.g., the ß- | TTACCAATGCTTAATCAGTGAGGCACCTATCTCAGCG |
| | lactamase gene) | ATCTGTCTATTTCGTTCATCCATAGTTGCCTGACTCCC |
| | | CGTCGTGTAGATAACTACGATACGGGAGGGCTTACC |
| | | ATCTGGCCCCAGTGCTGCAATGATACCGCGGGACCC |
| | | ACGCTCACCGGCTCCAGATTTATCAGCAATAAACCAG |
| | | CCAGCCGGAAGGGCCGAGCGCAGAAGTGGTCCTGC |
| | | AACTTTATCCGCCTCCATCCAGTCTATTAATTGTTGCC |
| | | GGGAAGCTAGAGTAAGTAGTTCGCCAGTTAATAGTTT |
| | | GCGCAACGTTGTTGCCATTGCTACAGGCATCGTGGT |
| | | GTCACGCTCGTCGTTTGGTATGGCTTCATTCAGCTCC |
| | | GGTTCCCAACGATCAAGGCGAGTTACATGATCCCCCA |
| | | TGTTGTGCAAAAAAGCGGTTAGCTCCTTCGGTCCTCC |
| | | GATGGTTGTCAGAAGTAAGTTGGCCGCAGTGTTATCA |
| | | CTCATGGTTATGGCAGCACTGCATAATTCTCTTACTGT |
| | | CATGCCATCCGTAAGATGCTTTTCTGTGACTGGTGAG |
| | | TACTCAACCAAGTCATTCTGAGAATAGTGTATGCGGC |
| | | GACCGAGTTGCTCTTGCCCGGCGTCAATACGGGATA |
| | | ATACCGCGCCACATAGCAGAACTTTAAAAGTGCTCAT |
| | | CATTGGAAAACGTTCTTCGGGGCGAAAACTCTCAAGG |
| | | ATCTTACCGCTGTTGAGATCCAGTTCGATGTAACCCA |
| | | CTCGTGCACCCAACTGATCTTCAGCATCTTTTACTTTC |
| | | ACCAGCGTTTCTGGGTGAGCAAAAACAGGAAGGCAA |
| | | AATGCCGCAAAAAAGGGAATAAGGGCGACACGGAAA |
| | | TGTTGAATACTCAT |

As described herein, an exemplary library expression vector including intergenic regions is as follows:

(SEQ ID NO: 11)
TTAAGACCCACTTTCACATTTAAGTTGTTTTTCTAATCCGCATATGATCAATTCAAGGCCGAATAAGAA
GGCTGGCTCTGCACCTTGGTGATCAAATAATTCGATAGCTTGTCGTAATAATGGCGGCATACTATCA
GTAGTAGGTGTTTCCCTTTCTTCTTTAGCGACTTGATGCTCTTGATCTTCCAATACGCAACCTAAAGTA
AAATGCCCCACAGCGCTGAGTGCATATAATGCATTCTCTAGTGAAAAACCTTGTTGGCATAAAAAGG
CTAATTGATTTTCGAGAGTTTCATACTGTTTTTCTGTAGGCCGTGTACCTAAATGTACTTTTGCTCCAT
CGCGATGACTTAGTAAAGCACATCTAAAACTTTTAGCGTTATTACGTAAAAAATCTTGCCAGCTTTCC
CCTTCTAAAGGGCAAAAGTGAGTATGGTGCCTATCTAACATCTCAATGGCTAAGGCGTCGAGCAAAG
CCCGCTTATTTTTTACATGCCAATACAATGTAGGCTGCTCTACACCTAGCTTCTGGGCGAGTTTACGG
GTTGTTAAACCTTCGATTCCGACCTCATTAAGCAGCTCTAATGCGCTGTTAATCACTTTACTTTTATCT
AATCTAGACATCATTAATTCCTAATTTTTGTTGACACTCTATCGTTGATAGAGTTATTTTACCACTCCCT
ATCAGTGATAGAGAAAAGAATTCAAAAGATCTAAAGAGGAGAAAGGATCTATGGATAAGAAATACTCA
ATAGGCTTAGCTATCGGCACAAATAGCGTCGGATGGGCGGTGATCACTGATGAATATAAGGTTCCGT
CTAAAAAGTTCAAGGTTCTGGGAAATACAGACCGCCACAGTATCAAAAAAAATCTTATAGGGGCTCTT
TTATTTGACAGTGGAGAGACAGCGGAAGCGACTCGTCTCAAACGGACAGCTCGTAGAAGGTATACA
CGTCGGAAGAATCGTATTTGTTATCTACAGGAGATTTTTTCAAATGAGATGGCGAAAGTAGATGATAG
TTTCTTTCATCGACTTGAAGAGTCTTTTTTGGTGGAAGAAGACAAGAAGCATGAACGTCATCCTATTTT
TGGAAATATAGTAGATGAAGTTGCTTATCATGAGAAATATCCAACTATCTATCATCTGCGAAAAAAATT
GGTAGATTCTACTGATAAAGCGGATTTGCGCTTAATCTATTTGGCCTTAGCGCATATGATTAAGTTTC
GTGGTCATTTTTTGATTGAGGGAGATTTAAATCCTGATAATAGTGATGTGGACAAACTATTTATCCAGT
TGGTACAAACCTACAATCAATTATTTGAAGAAAACCCTATTAACGCAAGTGGAGTAGATGCTAAAGCG
ATTCTTTCTGCACGATTGAGTAAATCAAGACGATTAGAAAATCTCATTGCTCAGCTCCCCGGTGAGAA
GAAAAATGGCTTATTTGGGAATCTCATTGCTTTGTCATTGGGTTTGACCCCTAATTTTAAATCAAATTT
TGATTTGGCAGAAGATGCTAAATTACAGCTTTCAAAAGATACTTACGATGATGATTTAGATAATTTATT
GGCGCAAATTGGAGATCAATATGCTGATTTGTTTTTGGCAGCTAAGAATTTATCAGATGCTATTTTACT
TTCAGATATCCTAAGAGTAAATACTGAAATAACTAAGGCTCCCCTATCAGCTTCAATGATTAAACGCTA
CGATGAACATCATCAAGACTTGACTCTTTTAAAAGCTTTAGTTCGACAACAACTTCCAGAAAAGTATAA
AGAAATCTTTTTTGATCAATCAAAAAACGGATATGCAGGTTATATTGATGGGGGAGCTAGCCAAGAAG
AATTTTATAAATTTATCAAACCAATTTTAGAAAAAATGGATGGTACTGAGGAATTATTGGTGAAACTAA
ATCGTGAAGATTTGCTGCGCAAGCAACGGACCTTTGACAACGGCTCTATTCCCCATCAAATTCACTT
GGGTGAGCTGCATGCTATTTTGAGAAGACAAGAAGACTTTTATCCATTTTTAAAAGACAATCGTGAGA
AGATTGAAAAAATCTTGACTTTTCGAATTCCTTATTATGTTGGTCCATTGGCGCGTGGCAATAGTCGT
TTTGCATGGATGACTCGGAAGTCTGAAGAAACAATTACCCCATGGAATTTTGAAGAAGTTGTCGATAA
AGGTGCTTCAGCTCAATCATTTATTGAACGCATGACAAACTTTGATAAAAATCTTCCAAATGAAAAAGT
ACTACCAAAACATAGTTTGCTTTATGAGTATTTTACGGTTTATAACGAATTGACAAAGGTCAAATATGT
TACTGAAGGAATGCGAAAACCAGCATTTCTTTCAGGTGAACAGAAGAAAGCCATTGTTGATTTACTCT
TCAAAACAAATCGAAAAGTAACCGTTAAGCAATTAAAAGAAGATTATTTCAAAAAAATAGAATGTTTTG
ATAGTGTTGAAATTTCAGGAGTTGAAGATAGATTTAATGCTTCATTAGGTACCTACCATGATTTGCTAA
AAATTATTAAAGATAAAGATTTTTTGGATAATGAAGAAAATGAAGATATCTTAGAGGATATTGTTTTAAC
ATTGACCTTATTTGAAGATAGGGAGATGATTGAGGAAAGACTTAAAACATATGCTCACCTCTTTGATG
ATAAGGTGATGAAACAGCTTAAACGTCGCCGTTATACTGGTTGGGGACGTTTGTCTCGAAAATTGATT
AATGGTATTAGGGATAAGCAATCTGGCAAAACAATATTAGATTTTTTGAAATCAGATGGTTTTGCCAAT
CGCAATTTTATGCAGCTGATCCATGATGATAGTTTGACATTTAAAGAAGACATTCAAAAAGCACAAGT
GTCTGGACAAGGCGATAGTTTACATGAACATATTGCAAATTTAGCTGGTAGCCCTGCTATTAAAAAAG
GTATTTTACAGACTGTAAAAGTTGTTGATGAATTGGTCAAAGTAATGGGGCGGCATAAGCCAGAAAAT
ATCGTTATTGAAATGGCACGTGAAAATCAGACAACTCAAAAGGGCCAGAAAAATTCGCGAGAGCGTA
TGAAACGAATCGAAGAAGGTATCAAAGAATTAGGAAGTCAGATTCTTAAAGAGCATCCTGTTGAAAAT
ACTCAATTGCAAAATGAAAAGCTCTATCTCTATTATCTCCAAAATGGAAGAGACATGTATGTGGACCA
AGAATTAGATATTAATCGTTTAAGTGATTATGATGTCGATGCCATTGTTCCACAAAGTTTCCTTAAAGA
CGATTCAATAGACAATAAGGTCTTAACGCGTTCTGATAAAAATCGTGGTAAATCGGATAACGTTCCAA
GTGAAGAAGTAGTCAAAAAGATGAAAAACTATTGGAGACAACTTCTAAACGCCAAGTTAATCACTCAA
CGTAAGTTTGATAATTTAACGAAAGCTGAACGTGGAGGTTTGAGTGAACTTGATAAAGCTGGTTTTAT
CAAACGCCAATTGGTTGAAACTCGCCAAATCACTAAGCATGTGGCACAAATTTTGGATAGTCGCATG
AATACTAAATACGATGAAAATGATAAACTTATTCGAGAGGTTAAAGTGATTACCTTAAAATCTAAATTA
GTTTCTGACTTCCGAAAAGATTTCCAATTCTATAAAGTACGTGAGATTAACAATTACCATCATGCCCAT
GATGCGTATCTAAATGCCGTCGTTGGAACTGCTTTGATTAAGAAATATCCAAAACTTGAATCGGAGTT
TGTCTATGGTGATTATAAAGTTTATGATGTTCGTAAAATGATTGCTAAGTCTGAGCAAGAAATAGGCA
AAGCAACCGCAAAATATTTCTTTTACTCTAATATCATGAACTTCTTCAAAACAGAAATTACACTTGCAA
ATGGAGAGATTCGCAAACGCCCTCTAATCGAAACTAATGGGGAAACTGGAGAAATTGTCTGGGATAA
AGGGCGAGATTTTGCCACAGTGCGCAAAGTATTGTCCATGCCCCAAGTCAATATTGTCAAGAAAACA
GAAGTACAGACAGGCGGATTCTCCAAGGAGTCAATTTTACCAAAAAGAAATTCGGACAAGCTTATTG
CTCGTAAAAAAGACTGGGATCCAAAAAAATATGGTGGTTTTGATAGTCCAACGGTAGCTTATTCAGTC
CTAGTGGTTGCTAAGGTGGAAAAAGGGAAATCGAAGAAGTTAAAATCCGTTAAAGAGTTACTAGGGA
TCACAATTATGGAAAGAAGTTCCTTTGAAAAAAATCCGATTGACTTTTTAGAAGCTAAAGGATATAAGG
AAGTTAAAAAAGACTTAATCATTAAACTACCTAAATATAGTCTTTTTGAGTTAGAAAACGGTCGTAAAC
GGATGCTGGCTAGTGCCGGAGAATTACAAAAAGGAAATGAGCTGGCTCTGCCAAGCAAATATGTGAA
TTTTTTATATTTAGCTAGTCATTATGAAAAGTTGAAGGGTAGTCCAGAAGATAACGAACAAAAACAATT
GTTTGTGGAGCAGCATAAGCATTATTTAGATGAGATTATTGAGCAAATCAGTGAATTTTCTAAGCGTG
TTATTTTAGCAGATGCCAATTTAGATAAAGTTCTTAGTGCATATAACAAACATAGAGACAAACCAATAC
GTGAACAAGCAGAAAATATTATTCATTTATTTACGTTGACGAATCTTGGAGCTCCCGCTGCTTTTAAAT
ATTTTGATACAACAATTGATCGTAAACGATATACGTCTACAAAAGAAGTTTTAGATGCCACTCTTATCC
ATCAATCCATCACTGGTCTTTATGAAACACGCATTGATTTGAGTCAGCTAGGAGGTGACGGTGGAGG
AGGTTCTCGATCGTGTACACGGGATCTCGACGCTCTCCCTTATGCGACTCCTGCATTAGGAAGCAGC
CCAGTAGTAGGTTGAGGCCGTTGAGCACCGCCGCCGCAAGGAATGGTGCATGCAAGGAGATGGCG
CCCAACAGTCCCCCGGCCACGGGGCCTGCCACCATACCCACGCCGAAACAAGCGCTCATGAGCCC
GAAGTGGCGAGCCCGATCTTCCCCATCGGTGATGTCGGCGATATAGGCGCCAGCAACCGCACCTGT
GGCGCCGGTGATGCCGGCCACGATGCGTCCGGCGTAGAGGATCGAGATCGTTTAGGCACCCCAGG
CTTTACACTTTATGCTTCCGGCTCGTATAATGTGTGGAATTGTGAGCGGATAACAATTTCAGAATTCA
AAAGATCTTTTAAGAAGGAGATATACATATGGCGAGTAGCGAAGACGTTATCAAAGAGTTCATGCGTT
TCAAAGTTCGTATGGAAGGTTCCGTTAACGGTCACGAGTTCGAAATCGAAGGTGAAGGTGAAGGTCG
TCCGTACGAAGGTACCCAGACCGCTAAACTGAAAGTTACCAAAGGTGGTCCGCTGCCGTTCGCTTG
GGACATCCTGTCCCCGCAGTTCCAGTACGGTTCCAAAGCTTACGTTAAACACCCGGCTGACATCCCG
GACTACCTGAAACTGTCCTTCCCGGAAGGTTTCAAATGGGAACGTGTTATGAACTTCGAAGACGGTG
GTGTTGTTACCGTTACCCAGGACTCCTCCCTGCAAGACGGTGAGTTCATCTACAAAGTTAAACTGCG
TGGTACCAACTTCCCGTCCGACGGTCCGGTTATGCAGAAAAAAACCATGGGTTGGGAAGCTTCCAC
CGAACGTATGTACCCGGAAGACGGTGCTCTGAAAGGTGAAATCAAAATGCGTCTGAAACTGAAAGAC
GGTGGTCACTACGACGCTGAAGTTAAAACCACCTACATGGCTAAAAAACCGGTTCAGCTGCCGGGT
GCTTACAAAACCGACATCAAACTGGACATCACCTCCCACAACGAAGACTACACCATCGTTGAACAGT
ACGAACGTGCTGAAGGTCGTCACTCCACCGGTGCTTAAGGATCCAAACTCGAGTAAGGATCTCCAG
GCATCAAATAAAACGAAAGGCTCAGTCGAAAGACTGGGCCTTTCGTTTTATCTGTTGTTTGTCGGTGA
ACGCTCTCTACTAGAGTCACACTGGCTCACCTTCGGGTGGGCCTTTCTGCGTTTATACCTAGGGATA
TATTCCGCTTCCTCGCTCACTGACTCGCTACGCTCGGTCGTTCGACTGCGGCGAGCGGAAATGGCT
TACGAACGGGGCGGAGATTTCCTGGAAGATGCCAGGAAGATACTTAACAGGGAAGTGAGAGGGCC
GCGGCAAAGCCGTTGGTCTCAGTTTTAGAGCTAGAAATAGCAAGTTAAAATAAGGCTAGTCCGTTAT
CAACTTGAAAAAGTGGCACCGAGTCGGTGCTTTTTTTGAAGCTTGGGCCCAGCCAGGAACCGTAAAA
AGGCCGCGTTGCTGGCGTTTTTCCACAGGCTCCGCCCCCCTGACGAGCATCACAAAAATCGACGCT
CAAGTCAGAGGTGGCGAAACCCGACAGGACTATAAAGATACCAGGCGTTTCCCCCTGGAAGCTCCC
TCGTGCGCTCTCCTGTTCCGACCCTGCCGCTTACCGGATACCTGTCCGCCTTTCTCCCTTCGGGAAG
CGTGGCGCTTTCTCATAGCTCACGCTGTAGGTATCTCAGTTCGGTGTAGGTCGTTCGCTCCAAGCTG
GGCTGTGTGCACGAACCCCCCGTTCAGCCCGACCGCTGCGCCTTATCCGGTAACTATCGTCTTGAG
TCCAACCCGGTAAGACACGACTTATCGCCACTGGCAGCAGCCACTGGTAACAGGATTAGCAGAGCG
AGGTATGTAGGCGGTGCTACAGAGTTCTTGAAGTGGTGGCCTAACTACGGCTACACTAGAAGAACA
GTATTTGGTATCTGCGCTCTGCTGAAGCCAGTTACCTTCGGAAAAAGAGTTGGTAGCTCTTGATCCG
GCAAACAAACCACCGCTGGTAGCGGTGGTTTTTTTGTTTGCAAGCAGCAGATTACGCGCAGAAAAAA
AGGATCTCAAGAAGATCCTTTGATCTTTTCTACGGGGTCTGACGCTCAGTGGAACGAAAACTCACGT
TAAGGGATTTTGGTCATGAGATTATCAAAAAGGATCTTCACCTAGATCCTTTTAAATTAAAAATGAAGT
TTTAAATCAATCTAAAGTATATATGAGTAAACTTGGTCTGACGTTACCAATGCTTAATCAGTGAGGCAC
CTATCTCAGCGATCTGTCTATTTCGTTCATCCATAGTTGCCTGACTCCCCGTCGTGTAGATAACTACG
ATACGGGAGGGCTTACCATCTGGCCCCAGTGCTGCAATGATACCGCGGGACCCACGCTCACCGGCT
CCAGATTTATCAGCAATAAACCAGCCAGCCGGAAGGGCCGAGCGCAGAAGTGGTCCTGCAACTTTA
TCCGCCTCCATCCAGTCTATTAATTGTTGCCGGGAAGCTAGAGTAAGTAGTTCGCCAGTTAATAGTTT
GCGCAACGTTGTTGCCATTGCTACAGGCATCGTGGTGTCACGCTCGTCGTTTGGTATGGCTTCATTC
AGCTCCGGTTCCCAACGATCAAGGCGAGTTACATGATCCCCCATGTTGTGCAAAAAAGCGGTTAGCT
CCTTCGGTCCTCCGATGGTTGTCAGAAGTAAGTTGGCCGCAGTGTTATCACTCATGGTTATGGCAGC
ACTGCATAATTCTCTTACTGTCATGCCATCCGTAAGATGCTTTTCTGTGACTGGTGAGTACTCAACCA
AGTCATTCTGAGAATAGTGTATGCGGCGACCGAGTTGCTCTTGCCCGGCGTCAATACGGGATAATAC
CGCGCCACATAGCAGAACTTTAAAAGTGCTCATCATTGGAAAACGTTCTTCGGGGCGAAAACTCTCA
AGGATCTTACCGCTGTTGAGATCCAGTTCGATGTAACCCACTCGTGCACCCAACTGATCTTCAGCAT
CTTTTACTTTCACCAGCGTTTCTGGGTGAGCAAAAACAGGAAGGCAAAATGCCGCAAAAAAGGGAAT
AAGGGCGACACGGAAATGTTGAATACTCATACTCTTCCTTTTTCAATATTATTGAAGCATTTATCAGG
GTTATTGTCTCATGAGCGGATACATATTTGAATGTATTTAGAAAAATAAACAAATAGGGGTTCCGCGC
ACATTTCCCCGAAAAGTGCCACCTGACGTC.

As described herein, an “expression scaffold” may be used for a subcloning step (e.g., a second subcloning step as seen in FIG. 3A and FIG. 5B for insertion of a universal C-terminal HA/6His on dCas9-fusion libraries, dCas9 terminator, CAT for chloramphenicol-based selection of desired clones, and/or T7 promoter for inducible sgRNA expression). As described herein, exemplary nucleic acid sequences that may be included in an expression scaffold are exemplified by the nucleic acid sequences in Table 2, shown below.

TABLE 2
Exemplary Nucleic Acid Sequences of an Expression Scaffold

| SEQ ID NO | Description | Sequence |
|---|---|---|
| 12 | SalI restriction | GTCGAC |
| | endonuclease site | |
| 13 | HA (e.g., HA tag) | TACCCGTACGACGTTCCGGACTACGCG |
| 14 | 6His (e.g., 6His tag) | CACCATCATCACCATCAT |
| 15 | Escherichia coli rrnB T1 | CAAATAAAACGAAAGGCTCAGTCGAAAGACTGGGCC |
| | terminator | TTTCGTTTTATCTGTTGTTTGTCGGTGAACGCTCTC |
| 16 | Chloramphenicol | TTACGCCCCGCCCTGCCACTCATCGCAGTACTGTTGT |
| | resistance gene (camR) | AATTCATTAAGCATTCTGCCGACATGGAAGCCATCAC |
| | | AAACGGCATGATGAACCTGAATCGCCAGCGGCATCA |
| | | GCACCTTGTCGCCTTGCGTATAATATTTGCCCATGGT |
| | | GAAAACGGGGGCGAAGAAGTTGTCCATATTGGCCAC |
| | | GTTTAAATCAAAACTGGTGAAACTCACCCAGGGATTG |
| | | GCTGAGACGAAAAACATATTCTCAATAAACCCTTTAG |
| | | GGAAATAGGCCAGGTTTTCACCGTAACACGCCACATC |
| | | TTGCGAATATATGTGTAGAAACTGCCGGAAATCGTCG |
| | | TGGTATTCACTCCAGAGCGATGAAAACGTTTCAGTTT |
| | | GCTCATGGAAAACGGTGTAACAAGGGTGAACACTATC |
| | | CCATATCACCAGCTCACCGTCTTTCATTGCCATACGA |
| | | AATTCCGGATGAGCATTCATCAGGCGGGCAAGAATGT |
| | | GAATAAAGGCCGGATAAAACTTGTGCTTATTTTTCTTT |
| | | ACGGTCTTTAAAAAGGCCGTAATATCCAGCTGAACGG |
| | | TCTGGTTATAGGTACATTGAGCAACTGACTGAAATGC |
| | | CTCAAAATGTTCTTTACGATGCCATTGGGATATATCAA |
| | | CGGTGGTATATCCAGTGATTTTTTTCTCCAT |
| 17 | SpeI restriction | TAATACGACTCACTATAG |
| | endonuclease restriction | |
| | site | |
| 18 | T7 promoter | ACTAGT |

As described herein, an exemplary expression scaffold including intergenic regions is as follows:

(SEQ ID NO: 19)
GTCGACTACCCGTACGACGTTCCGGACTACGCGGGTGGTCACCATCATC
ACCATCATTAATAAGGATCTCCAGGCATCAAATAAAACGAAAGGCTCAG
TCGAAAGACTGGGCCTTTCGTTTTATCTGTTGTTTGTCGGTGAACGCTC
TCTACTAGAGTCACACTGGCTCACCTTCGGGTGGGCCTTTCTGCGTTTA
TACCTAGGGATATATTCCGCTGCTTGGATTCTCACCAATAAAAAACGCC
CGGCGGCAACCGAGCGTTCTGAACAAATCCAGATGGAGTTCTGAGGTCA
TTACTGGATCTATCAACAGGAGTCCAAGCGAGCTCGATATCAAATTACG
CCCCGCCCTGCCACTCATCGCAGTACTGTTGTAATTCATTAAGCATTCT
GCCGACATGGAAGCCATCACAAACGGCATGATGAACCTGAATCGCCAGC
GGCATCAGCACCTTGTCGCCTTGCGTATAATATTTGCCCATGGTGAAAA
CGGGGGCGAAGAAGTTGTCCATATTGGCCACGTTTAAATCAAAACTGGT
GAAACTCACCCAGGGATTGGCTGAGACGAAAAACATATTCTCAATAAAC
CCTTTAGGGAAATAGGCCAGGTTTTCACCGTAACACGCCACATCTTGCG
AATATATGTGTAGAAACTGCCGGAAATCGTCGTGGTATTCACTCCAGAG
CGATGAAAACGTTTCAGTTTGCTCATGGAAAACGGTGTAACAAGGGTGA
ACACTATCCCATATCACCAGCTCACCGTCTTTCATTGCCATACGAAATT
CCGGATGAGCATTCATCAGGCGGGCAAGAATGTGAATAAAGGCCGGATA
AAACTTGTGCTTATTTTTCTTTACGGTCTTTAAAAAGGCCGTAATATCC
AGCTGAACGGTCTGGTTATAGGTACATTGAGCAACTGACTGAAATGCCT
CAAAATGTTCTTTACGATGCCATTGGGATATATCAACGGTGGTATATCC
AGTGATTTTTTTCTCCATTTTAGCTTCCTTAGCTCCTGAAAATCTCGAT
AACTCAAAAAATACGCCCGGTAGTGATCTTATTTCATTATGGTGAAAGT
TGGAACCTCTTACGTGCCGATCAACGTCTCATTTTCGCCAGATATCGAC
GTCCTAAAGATCTAATACGACTCACTATAGACTAGT.

### EXAMPLE 2

CasPlay: qRNA-Barcoded CRISPR-Based Display Platform for Antibody Repertoire Profiling

Protein display technologies link proteins to distinct nucleic acid sequences (barcodes), enabling multiplexed protein assays via DNA sequencing. Here, we developed Cas9 display (CasPlay)(also referred to as “CRISPR-based protein display using sgRNA sequencing”) to interrogate customized peptide libraries fused to catalytically inactive Cas9 (dCas9) by sequencing the guide RNA (gRNA) barcodes associated with each peptide (gRNA sequences are amplified by RT-PCR and barcode abundances are tracked by next-generation sequencing (NGS)). We first confirmed the ability of CasPlay to characterize antibody epitopes by recovering a known binding motif for a monoclonal anti-FLAG antibody. We then use a CasPlay library tiling the SARS-CoV-2 proteome to evaluate vaccine-induced antibody reactivities. We performed immunoprecipitations using monoclonal antibodies and human serum samples, showing that CasPlay can be used to identify antibody specificities by detecting the enrichment of certain peptide species with gRNA barcode sequencing. We also performed an experiment to illustrate the compatibility of CasPlay with synthetic antibody presentation for analyte detection experiments. Using a peptide library representing the human virome, we demonstrated the ability of CasPlay to identify epitopes across many viruses from microliters of patient serum. Our results indicate that CasPlay is a viable strategy for customized protein interaction studies from highly complex libraries and could provide an alternative to phage display technologies.

CasPlay advantageously provides a versatile approach to catalogue protein interactions with potential for diverse research and diagnostics applications.

### Results

### CasPlay Uncovers Known Anti-FLAG Antibody Peptide Binding Motif

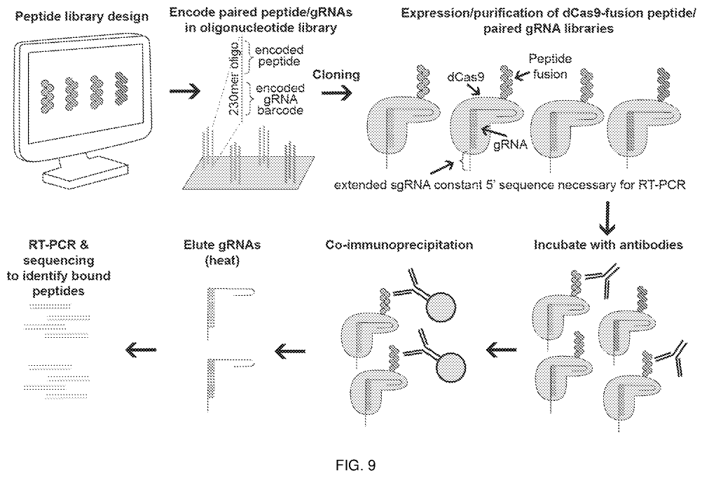

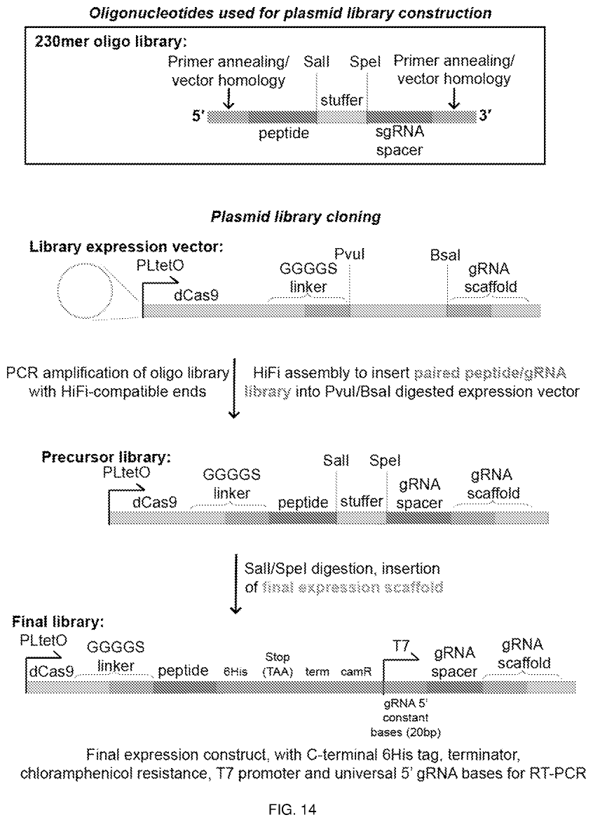

To perform CasPlay experiments, we first design peptide sequences encoded on the same strand of DNA as an orthogonalized 20 nt barcode (FIG. 9). The DNA library is then cloned in a single pool into an expression vector for the E. coli-based production of the peptide library as C-terminal fusions to dCas9 bound to a gRNA barcode that serves as a unique identifier for the peptide (FIG. 14) (Barber et al., Mol Cell 81, 3650-3658, 2021). To facilitate sequencing of gRNAs for CasPlay, we added a 20 nt universal sequence to the 5′ end of the gRNAs to permit amplification by RT-PCR. We then use the CasPlay library for immunoprecipitation experiments by incubating the peptides with a collection of antibodies. gRNA sequences are then amplified and identified by NGS, and subsequent barcode enrichment analysis reveals peptides that were bound to the antibodies.

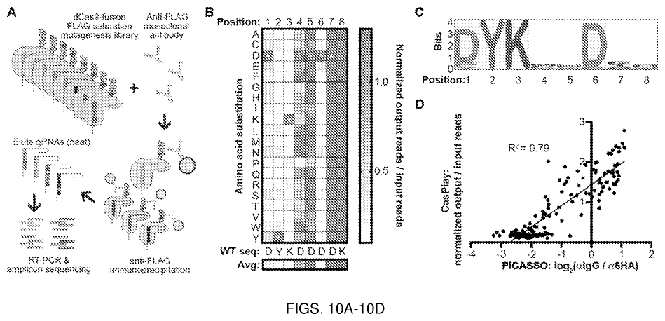

In one example of the CasPlay methodology we characterized antibody-epitope binding. We first constructed a dCas9-displayed FLAG peptide saturation mutagenesis library encompassing all 152 possible single amino acid substitutions along the of the length of the FLAG epitope (DYKDDDDK, FIG. 10A). We incubated this CasPlay library with the anti-FLAG M2 antibody, whose binding to the FLAG epitope has been extensively characterized (Layton et al., Mol Cell 73, 1075-1082, 2019). After immunoprecipitation, we amplified the gRNA sequences by RT-PCR and performed NGS to identify the barcodes of FLAG variant peptides that bound the M2 antibody (FIG. 101B). We successfully recapitulated a DYKxxDxx binding motif of the M2 antibody (Osada et al., J Biochem 145, 693-700, 2009; Srila and Yamabhai, Appl Biochem Biotech 171, 583-589, 2013), demonstrating the capability of CasPlay to identify amino acid residues that are critical to coordinate antibody binding (FIG. 10C). The barcode enrichment analysis performed comparably to PICASSO experiments using the same dCas9-displayed FLAG variant library with M2 and a fluorescence-based microarray scanning assay (Barber et al., 2021) (R2=0.79, FIG. 10D), showing the ability of NGS-based gRNA sequencing to identify antibody-bound peptides.

Epitopes Associated with SARS-CoV-2 Infection or Vaccination Observed by CasPlay

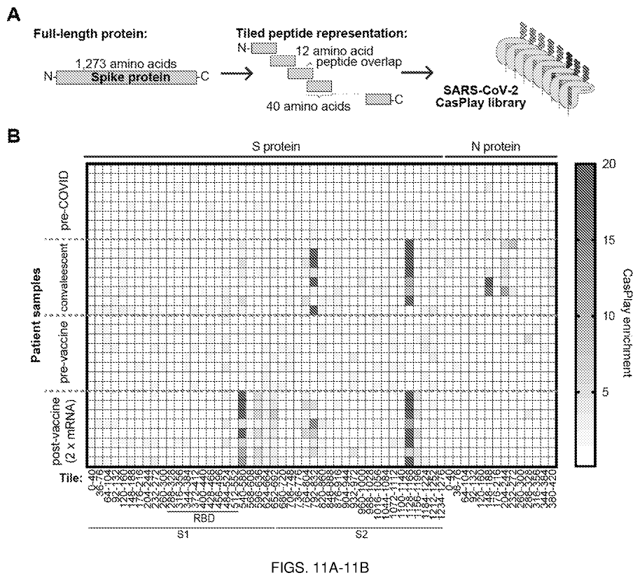

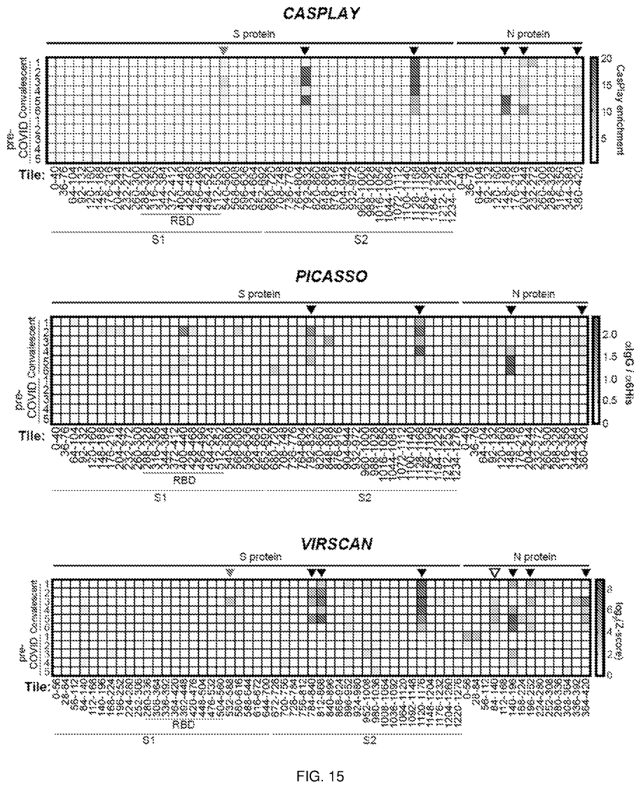

We then constructed a CasPlay library consisting of 40mer peptide tiles representing proteins from SARS-CoV-2 (FIG. 11A). We incubated this library with serum collected from patients previously infected with SARS-CoV-2 (n=8) as well as human serum samples from before the beginning of the COVID-19 global pandemic (n=8) and identified the peptides bound to patient antibodies using gRNA barcode sequencing. We were able to identify several regions from across the spike (S:540-580; S:764-804; S:792-832; S:1128-1168) and nucleocapsid (N:148-188; N:204-244; N:380-420) proteins that were specifically enriched in at least two convalescent patient samples (barcode enrichment 3.5, FIG. 11B). Comparing patient-matched results between CasPlay and PICASSO, CasPlay revealed one additional epitope that was not observed by PICASSO (N: 204-244, FIG. 15). CasPlay identified all the same shared epitopes observed using phage display experiments except one in the nucleocapsid protein (N:92-132, FIG. 15), potentially due to differences between the platforms in epitope context or displayed copy number.

Using the same CasPlay library, we also evaluated patient antibody reactivities elicited in response to SARS-CoV-2 mRNA vaccination (n=8, FIG. 11B). Antibodies from vaccinated individuals exhibited binding to peptides from the spike protein but not the nucleocapsid since the mRNA vaccine encodes only the spike protein (Turner et al., Nature 596, 109-113, 2021). Furthermore, antibodies toward additional epitopes in the C-terminal domain of the S1 portion of the spike protein (S:596-636; S:652-692) were observed only in response to the vaccine and not in SARS-CoV-2 infected patients, consistent with previous studies (Garrett et al., Elife 11, e73490, 2022). CasPlay thereby enables nuanced characterization of differences in antibody repertoire between patients and could be used to stratify patients that were vaccinated against or naturally infected by SARS-CoV-2.

### Human Virome Displayed by CasPlay Enables Antibody Epitope Localization Simultaneously Across Many Viruses

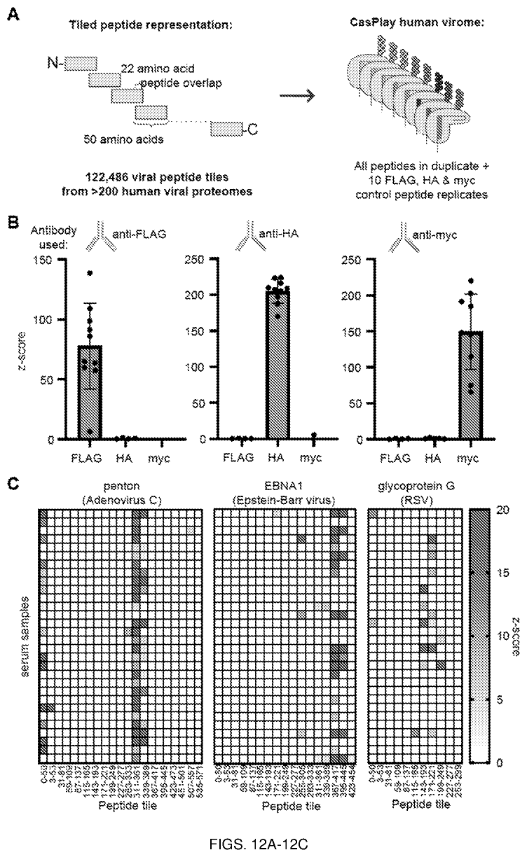

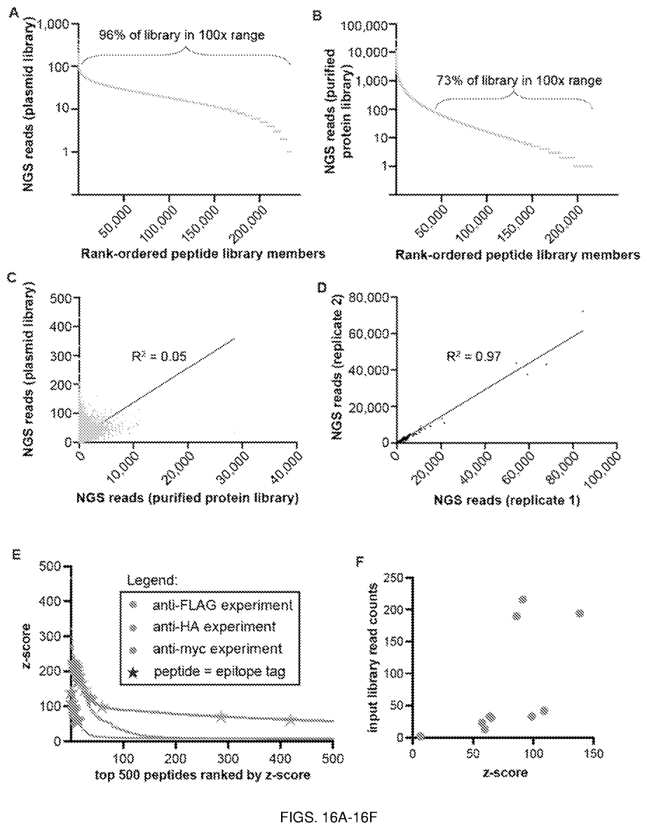

We then expanded the CasPlay library to encode a peptide-based representation of the entire human virome (FIG. 12A). We created a dCas9-displayed library of 50mer peptides overlapping by 22 amino acids, tiled along the lengths of proteins derived from all viruses known to infect humans. Each of the 122,486 encoded peptides was encoded in duplicate and paired with a unique gRNA barcode. Within the library, we also encoded peptides that are the targets of commercial monoclonal antibodies (FLAG, HA, and myc) for control experiments. NGS demonstrated that the CasPlay virome plasmid library was at least 96% complete, with approximately 96% of the gRNA barcodes falling within a 100-fold range of abundance (FIG. 16A). Sequencing of the gRNAs after RT-PCR from the purified dCas9-fusion library exhibited more sequence skewing, with 73% of the barcodes falling within a 100-fold range of abundance and only evidence for at least 88% of the encoded sequences, though deeper sequence could reveal great completion (FIG. 16B). No correlation was observed between the gRNA barcode read counts in the plasmid library and the gRNAs from the purified protein library (FIG. 16C), indicating that other peptide- or gRNA-specific factors affect final library abundance. Between independent preparations of the purified CasPlay library, relative barcode read counts were highly reproducible (R2=0.97, FIG. 16D).

To initially evaluate the performance of CasPlay for studies using the virome-wide library, we performed immunoprecipitations using anti-FLAG, anti-HA, and anti-myc monoclonal antibodies. gRNA barcode sequencing analysis revealed the selective enrichment of all ten replicates of each of the anticipated epitopes for each tested antibody (FIG. 12B). Within the entire CasPlay virome library (theoretical complexity of 245,002 barcodes), the barcodes for all replicate epitope tag peptides were ranked in the top 0.2% of z-scores for all peptides in experiments using their corresponding target antibodies (FIG. 16E). Since the HA epitope tag (YPYDVPDYA) is derived from a viral protein (influenza hemagglutinin), 98 viral peptides within the library (including replicates) also contained this sequence, of which 94 scored within the top 0.2% by z-score in the anti-HA immunoprecipitation. We observed that the lowest-ranking FLAG replicate peptide by z-score in the anti-FLAG immunoprecipitation experiment had a very low read count in the purified CasPlay “input” protein library, suggesting that peptides that are of low abundance in the library may affect results interpretation for those sequences (FIG. 16F).

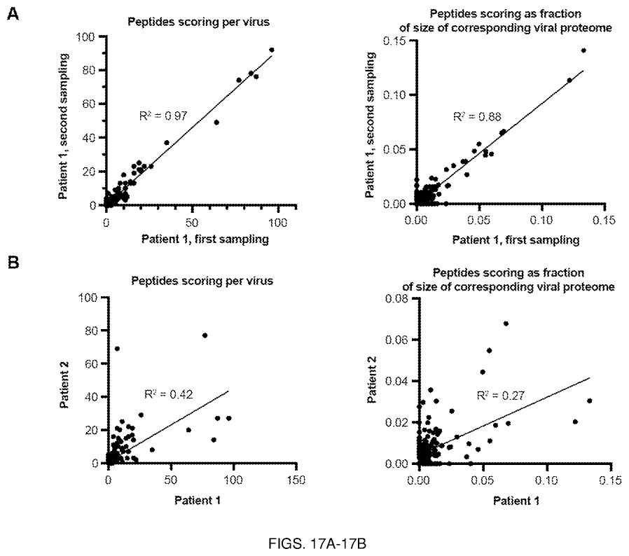

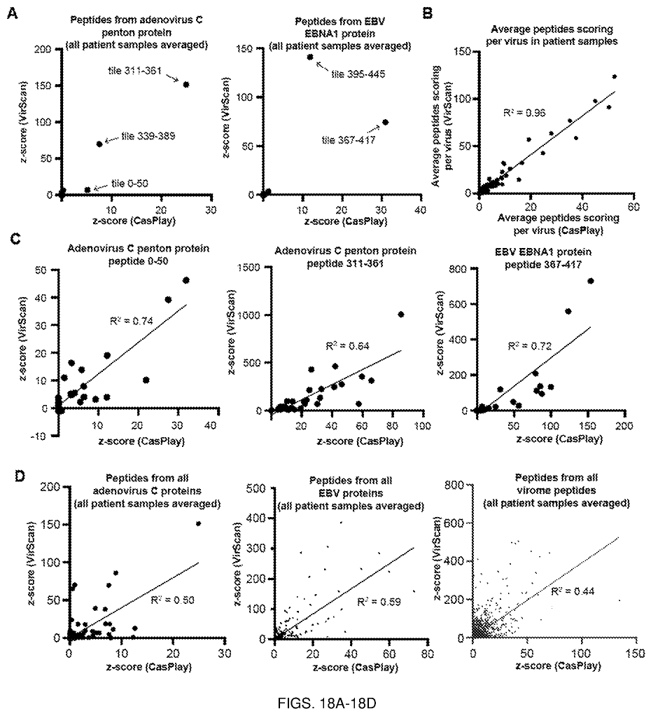

We then performed immunoprecipitation experiments using the virome-wide CasPlay library with 30 human serum samples. As a benchmark of reproducibility, we looked at the total number of peptides that scored per virus in two patient-matched longitudinal samples and observed a strong correlation (R2=0.97, FIG. 17A) compared to two unrelated patient samples (R2=0.42, FIG. 17B). From our dataset, we looked for epitopes across common viruses that were widely recognized across patient samples (FIG. 12C). We observed convergent antibody responses to peptides within the penton protein of adenovirus C (0:50; 311-361; 339-389), Epstein-Barr virus (EBV) EBNA1 protein (367-417; 395-445), and respiratory syncytial virus (RSV) glycoprotein G (143-193; 171-221), all of which have been observed as public epitopes using VirScan (FIG. 18A) (Xu et al., Science 348, aaa0698, 2015).

To further evaluate CasPlay's performance, we performed comparative analysis using the same patient samples by VirScan. The average number of peptides scoring per virus in each patient sample (z-score ≥3.5) correlated very well between VirScan and CasPlay (R2=0.96), though VirScan detected on average approximately 2-fold more peptide hits per virus (FIG. 18B). The viruses with the most detected peptide-reactive antibodies per patient sample (including RSV, rhinoviruses and enteroviruses) are ranked similarly between CasPlay and VirScan (Table 3). Across all tested patients, the z-scores for the public epitopes from adenovirus C and Epstein-Barr virus correlated between CasPlay and VirScan (FIG. 18C). Across the entire proteomes of adenovirus C and EBV as well as the virome library as a whole, the average patient z-score per peptide correlated more weakly between the two platforms (R2=0.50, 0.59, and 0.44 respectively, FIG. 18D). The decreased detection of certain peptide hits by CasPlay as well as differences in z-scores may be partially attributed to peptides with lower input counts in the library, as observed in FIG. 16F.

Table 3: Top 10 Viruses with the Most Relative Peptide Hits by CasPlay and VirScan

The average number of peptide hits (z-score ≥3.5) per virus per patient sample, reported as number of peptides or number of peptides as a percentage of the number of all peptides derived from that virus in the library by CasPlay and VirScan. Viruses are sorted by average peptide hits in CasPlay as a percentage of the viral proteome size, with viruses with fewer than 100 proteome peptides removed.

| | | | Total | CasPlay - | VirScan - |
|---|---|---|---|---|---|
| | | | peptides from | peptide hits as | peptide hits as |
| | CasPlay - | VirScan - | virus | percentage of | percentage of |
| | average | average | encoded in | encoded viral | encoded viral |
| Virus | peptide hits | peptide hits | library | proteome | proteome |
| Human respiratory | 52.5 | 123.7 | 781 | 6.7 | 15.8 |
| syncytial virus | | | | | |
| Rhinovirus B | 16.7 | 32.3 | 257 | 6.5 | 12.6 |
| Rhinovirus A | 35.2 | 76.9 | 660 | 5.3 | 11.7 |
| Enterovirus B | 45.1 | 97.8 | 1336 | 3.4 | 7.3 |
| Enterovirus C | 28.0 | 63.6 | 1009 | 2.8 | 6.3 |
| Human | 4.3 | 8.9 | 169 | 2.6 | 5.3 |
| parvovirus B19 | | | | | |
| Human | 50.4 | 91.3 | 1980 | 2.5 | 4.6 |
| herpesvirus 4 | | | | | |
| Human | 10.5 | 18.9 | 652 | 1.6 | 2.9 |
| adenovirus C | | | | | |
| Human | 24.8 | 42.7 | 1606 | 1.5 | 2.7 |
| herpesvirus 1 | | | | | |
| Influenza B | 9.4 | 32.4 | 875 | 1.1 | 3.7 |
| virus | | | | | |

Full-Length Functional Synthetic Antibodies are Compatible with CasPlay

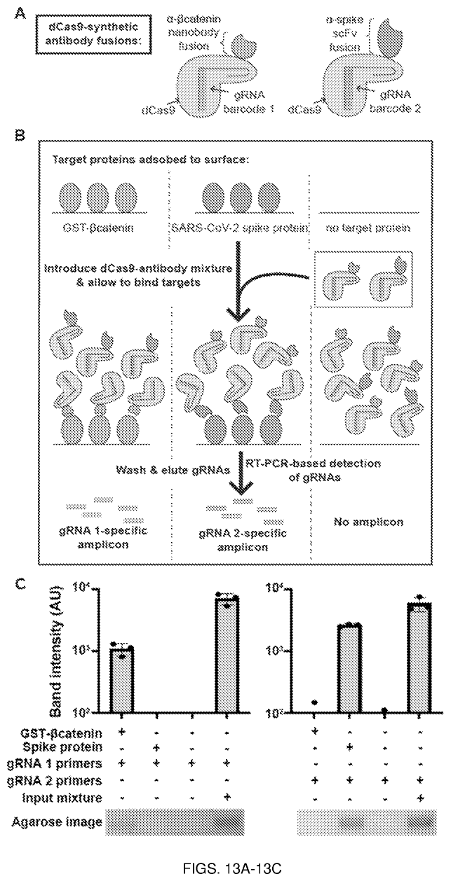

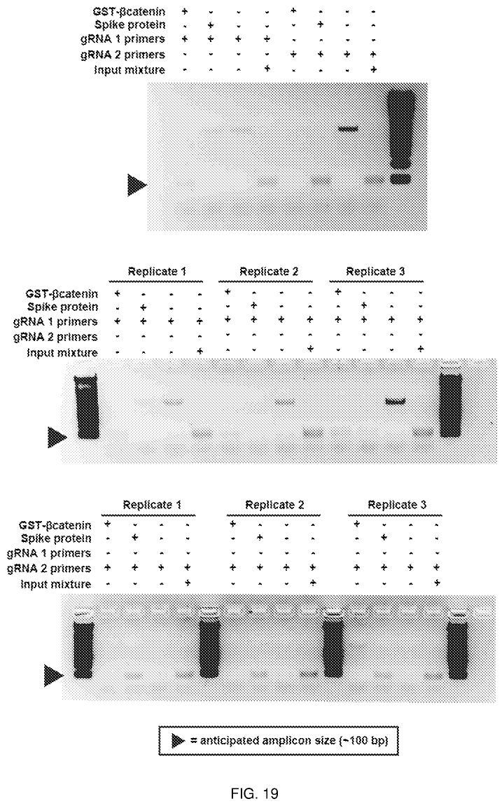

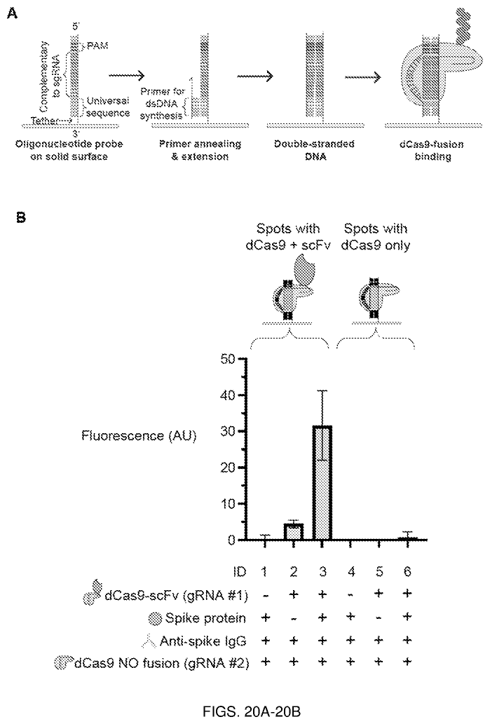

Finally, we also determined whether CasPlay is compatible with the display of longer folded proteins. To this end, we fused two classes of synthetic antibodies to dCas9: a nanobody recognizing β-catenin (Braun et al., Sci Rep-Uk 6, 19211, 2016; Traenkle et al., Mol Cell Proteomics 14, 707-723, 2015) and an scFv that binds the spike protein from SARS-CoV-2 (Wang et al., Science 373, 2021) (FIG. 13A). Each antibody was associated with a different gRNA barcode and purified from E. coli. To test the functionality of these antibodies, we immobilized target antigens (GST fused to the β-catenin epitope tag or recombinant SARS-CoV-2 spike protein) in separate wells of an adsorbent microplate. After applying a mixture of the two dCas9-fused antibodies, we washed away unbound protein and assessed the presence of each antibody using gRNA-specific primers for RT-PCR (FIG. 13B). We observed retention of only the cognate synthetic antibody for each antigen using primers specific to the corresponding gRNA barcode (FIG. 130, FIG. 19), showing that each antibody selectively bound its anticipated target and demonstrating the amenability of longer, folded proteins such as synthetic antibodies to CasPlay. We also note that this type of antibody fusion can also be used for analyte detection via dCas9-immobilization by PICASSO (demonstrated in FIG. 20).

In the results above, we have illustrated the ability of dCas9-displayed peptides and proteins to be used for protein interaction studies using a simple gRNA-based sequencing readout. CasPlay pinpoints amino acid positions within peptides coordinating antibody-epitope interactions as well as locate the epitopes of human serum antibodies within the context of larger proteins. We have also shown the ability of CasPlay using a very large (245,002) peptide library representing the human virome to identify epitopes across diverse viruses.

The above results were obtained using the following materials and methods.

### Experimental Model and Subject Details

### Microbe Strains

Plasmid and plasmid library cloning was performed in ElectroMAX DH10B E. coli cells (Thermo Fisher) grown at 37° C. dCas9-fusion libraries were expressed in T7 Express lysY Competent E. coli (High Efficiency, NEB) grown at 37° C. Further information about expression conditions are included in the “Method details” section below.

### Human Samples

COVID-19 convalescent samples and healthy controls were collected and analyzed by VirScan in previous studies (Shrock et al., Science 370, eabd4250, 2020). Eight deidentified exempt blood samples were used for the pre- and post-vaccine cohort analyzed in this study.

### Method Details

Design of CasPlay dCas9-Peptide Fusion Libraries and Synthetic Antibody Fusions

The dCas9-fusion peptides used for anti-FLAG M2 antibody epitope binding characterization and for targeted SARS-CoV-2 epitope mapping experiments were designed and described (Barber et al., Mol Cell 81, 3650-3658, 2021).

The human virome peptide library was designed based on previous phage display libraries (120,396 peptides from viruses that infect humans (Xu et al., Science 348, aaa0698, 2015) plus 1,794 coronavirus-derived peptides (Shrock et al., Science 370, eabd4250, 2020). In these prior studies, 56mer peptides tiling viral proteins with 28 amino acid overlap between adjacent tiles were presented on T7 phage. These peptides were used as the basis for the design of the 50mer peptides used in CasPlay, centered around the same residues as the 56mer peptides (i.e. the peptides presented by CasPlay were 3 amino acids shorter on both the N- and C-termini, and adjacent tiles overlapped by 22 amino acids). Additional 50mer peptides were included to encompass the N- and C-termini of each protein. 292 peptides representing SARS-CoV-2 variants with United States Centers for Disease Control and Prevention designations “being monitored” and “of concern” (www.cdc.gov/coronavirus/2019-ncov/variants/variant-classifications.html) as of January 2022 were also included in the library; for these peptides, the amino acid substitutions and deletions occurring in the viral variant proteins were incorporated in the corresponding peptide tiles, such that the register of the tiles was not altered from the original SARS-CoV-2 library peptide tiles to enable binding comparisons between variant peptides. Control peptide epitope tags, including HA, myc and FLAG, were also included in the library.

### Oligonucleotide Library Design & Cloning

The CasPlay-compatible 50mer viral peptides were codon optimized for expression in E. coli by mimicking natural codon frequency with rare codons removed (Xu et al., Science 348, aaa0698, 2015). Each peptide was encoded in duplicate (separately codon optimized), with the exception of the monoclonal epitope tag controls (HA, myc and FLAG), which were encoded with 10 replicates. Each peptide was associated with a unique, synthetic gRNA sequence that differed from every other gRNA sequence by at least 1 base pair within the first 10 bases from the 3′ end of the spacer sequence; the remaining 10 bases were randomized, with the stipulations that extraneous protospacer adjacent motifs (“CCN”) and polyT sequences (“TTTT”) be removed. Each gRNA sequence was additionally ensured to have a minimum Levenshtein distance of 3 from every other sequence within the library (Zorita et al., Bioinformatics 31, 1913-1919, 2015). gRNA spacer sequences with the lowest degree of predicted secondary structure (Hofacker, Nucleic Acids Res 31, 3429-3431, 2003) were then selected from this set. Each peptide replicate was associated with a unique gRNA sequence.

The oligonucleotides contained the following, from 5′ to 3′: homology arm for Gibson assembly (5′-GAGGAGGTTCTCGATCG-3′ (SEQ ID NO: 28)); peptide-encoding region; Sall restriction site; randomized bases to make total oligo length 230 bp (only included for peptides shorter than 50 amino acids, such as epitope tag controls); additional A base; XhoI restriction site; additional A base; SpeI restriction site; gRNA spacer sequence; homology arm for Gibson assembly (5′-GTTTTAGAGCTAGAAATAGCAAG-3′ (SEQ ID NO: 29). The 245,004 230mer oligonucleotides were synthesized across two equal-sized pools by Agilent Technologies.

Primers complementary to the homology arms within the oligonucleotides were used for library amplification using Q5 polymerase (NEB) on a 50 μL scale with 100 fmol of the oligonucleotide library template, 59° C. annealing temperature, and 60 s extension time with a total of 10 amplification cycles. PCR products were desalted using a PCR clean-up spin column (Machery-Nagel). The library amplicon was introduced into the BsaI/PvuI-digested precursor vector (Addgene #171798) using 80 ng vector backbone and 20 ng amplified oligonucleotide library insert, in a 10 μL total HiFi reaction (NEB). After incubation at 50° C. for 1 h, the DNA was desalted using 0.7× Ampure XP beads (Beckman Coulter) and transformed into 20 μL ElectroMAX DH10B cells (Thermo Fisher). This was performed in quadruplicate for each of the two Agilent oligonucleotide pools. After 1 h recovery in 1 mL SOC at 37° C., cells were spread on 15 cm LB+100 μg/mL carbenicillin plates. The following morning, cells were scraped and miniprepped to harvest the vector libraries.

For the second library subcloning step (for tiled human virome and targeted FLAG saturation mutagenesis and SARS-CoV-2 epitope libraries), an insert encoding a 6His tag, stop codon, transcriptional terminator, chloramphenicol resistance marker, T7 promoter for gRNA expression, and 5′ gRNA constant region for RT-PCR-based amplification was amplified from a previously described vector (Addgene #171799) (Barber et al., Mol Cell 81, 3650-3658, 2021) using the following primers: 5′-GGAAGAGTCGACCACCATC-3′ (SEQ ID NO: 30) and 5′-CAACCAACACTAGTACGTAGTCTGTACCTGATCTCTATAGTGAGTCGTATTAGATCTTTAGGACGTCG ATATCTG-3′ (SEQ ID NO: 31). This insert amplicon and the precursor plasmid library detailed above were digested with Sall and SpeI. 100 ng of the digested library backbone was ligated with 50 ng of digested insert using T4 DNA ligase (NEB). 10 replicate ligation reactions were performed for each library. The ligations were desalted using 0.7× Ampure beads (Beckman Coulter) and transformed into 20 μL ElectroMAX DH10B cells (Thermo Fisher). After 1 h recovery in 1 mL SOC at 37° C., cells were spread on 15 cm LB+100 μg/mL carbenicillin+50 μg/mL chloramphenicol plates. The following morning, cells were scraped and miniprepped to harvest the vector libraries.

CasPlay dCas9-Fusion Library Expression and Purification

100 ng of the final plasmid library was transformed into T7 Express lysY E. coli (NEB). After 1 h recovery in 1 mL LB at 37° C., cells were inoculated into 50 mL LB+100 μg/mL carbenicillin+50 μg/mL chloramphenicol and grown at 37° C. for 16 hours. Cells were then diluted to OD600=0.2 in 250 mL LB+100 μg/mL carbenicillin+50 μg/mL chloramphenicol, shaking at 225 rpm. The four sublibraries for the targeted FLAG saturation mutagenesis experiments were combined at this stage. Separately, the four sublibraries for the SARS-CoV-2 epitope mapping experiments were also combined at this stage. The two halves of the human virome library were not combined and were isolated in parallel. When cells reached OD600=0.8, dCas9-fusion peptide and gRNA expression were induced with 100 ng/mL anhydrotetracycline (ATC) and 0.1 mM IPTG, respectively. Cells were grown at 37° C., 225 rpm for an additional 4 h. Cells were harvested by centrifugation and pellets were stored at −80° C. for at least 12 h.

Once thawed, cell pellets were resuspended in 12.5 mL lysis buffer containing 50 mM Tris pH 7.5, 500 mM NaCl, 10% glycerol, 100 μM DTT, 5 μL rLysozyme solution (Millipore Sigma), 25 μL benzonase (90% purity, Millipore Sigma), 1× BugBuster (Millipore Sigma), and 1× protease inhibitors (cOmplete EDTA-free, Millipore Sigma), rotating at 25° C. for 30 min. Clarified lysates were incubated with 250 μL bed volume equilibrated Ni-NTA agarose (Qiagen) for 30 min at 23° C., rotating end-over-end. Resin was washed twice with 2.5 mL wash buffer (50 mM Tris pH 7.4, 500 mM NaCl, 10% glycerol, 100 μM DTT, 20 mM imidazole), and dCas9-fusions complexed with gRNAs were eluted using 2×250 μL elution buffer (50 mM Tris pH 7.4, 500 mM NaCl, 10% glycerol, 100 μM DTT, 500 mM imidazole). Eluates were passed through a 45 μm filter and buffer exchanged using a 100 kDa molecular weight cutoff Amicon Ultra-4 centrifugal filter (Millipore Sigma) with storage buffer (50 mM Tris pH 7.4, 150 mM NaCl, 10% glycerol, 1 mM DTT). Protein concentration was estimated by A260 and protein was stored at −20° C.

CasPlay dCas9-Fusion Library Precipitation and Sequencing

Convalescent serum and samples from before December 2020 were described previously (Shrock et al., Science 370, eabd4250, 2020). Deidentified longitudinal vaccine cohort samples of individuals with no know prior SARS-CoV-2 infection were collected prior to SARS-CoV-2 vaccination as well as between two weeks and three months after administration of the second dose of either Pfizer or Moderna mRNA vaccine. Patient serum samples were diluted 1:50 in PBS. 10 μL diluted serum was transferred into a 96 well plate and mixed with 20 ng of dCas9-6His bound to a gRNA lacking the 5′ overhang necessary for RT-PCR, 1% bovine serum albumin (BSA, w/v), and 250 μg/mL salmon sperm DNA (ThermoFisher), diluted to 60 μL total volume in TBST. For experiments using monoclonal antibodies, 1 μg of antibody was used (anti-FLAG M2 Millipore Sigma F1804; anti-HA Cell Signaling C29F4; anti-myc Abcam ab9106). Samples were incubated mixing end-over-end for 30 min at ambient temperature. Approximately 1 μg dCas9-fusion library was then mixed with each sample. For experiments using the tiled human virome, the two purified dCas9-fusion library subpools were combined prior to addition to the serum samples. Control experiments lacking serum or monoclonal antibodies were also performed to assess dCas9-fusion library background binding to the beads. Samples were then incubated at ambient temperature mixing end-over-end for 1 h. 20 μL of protein A Dynabeads (ThermoFisher) and 20 μL of protein G Dynabeads (ThermoFisher) were then added to each sample. Samples were then incubated for 16 h at 4° C., rotating end-over-end. Samples were then washed with 6×100 μL TBST on a magnet plate. Beads were then resuspended in 10 μL water and heated to 95° C. for 5 min to elute gRNAs. 6.5 μL of eluate was used for reverse transcription with SuperScript IV (ThermoFisher) using the manufacturer-suggested protocol on a 0.5× scale with primer 5′-GCACCGACTCGGTGCCACTTTTTC-3′ (SEQ ID NO: 32). Samples were then amplified using primers 5′-AGATCAGGTACAGACTACGTACTAG-3′ (SEQ ID NO: 33) and 5′-GCACCGACTCGGTGC-3′ (SEQ ID NO: 34) with Q5 polymerase (NEB) at 65° C. with 20 s extension time and 45 amplification cycles. Adapters for pooled Illumina sequencing were appended by PCR as previously described (Larman et al., Nat Biotechnol 29, 535-541, 2011; Xu et al., Science 348, aaa0698, 2015). Pooled gRNA amplicons were sequenced using an Illumina NextSeq 500 with approximately 2 million single-end 150 bp reads per sample.

### CasPlay Data Analysis

From NGS reads of gRNA amplicons, constant regions on sequencing reads surrounding the gRNA barcodes were removed using Cutadapt v2.5 (Martin, Embnet J 17, 10-12, 2011). Raw read counts were obtained by assigning each sequencing read to an encoded gRNA barcode if the sequence was a perfect match to an anticipated barcode (20/20 correct bases) and associating the sequence with its paired peptide. For analysis of CasPlay FLAG saturation mutagenesis and SARS-CoV-2 peptide libraries, gRNA barcode normalized counts after immunoprecipitation were divided by the normalized read counts in the purified “input” dCas9-fusion library to calculate enrichment. Enrichment values for each replicate peptide were averaged for all gRNA barcodes with at least 50 or 100 raw read counts in the “input” sample in the FLAG and SARS-CoV-2 experiments, respectively. For SARS-CoV-2 experiments in FIG. 11, results were further normalized by dividing the enrichment score of each peptide by the average score of the same peptide from the pre-COVID samples. Values for all analysis were averaged for two independent antibody sample replicates. For the CasPlay virome-wide library, gRNA barcodes from the purified “input” protein libraries used for experiments were rank-ordered based on read counts and divided into 300-member bins with the top and bottom 5% of sequences excluded. gRNA barcodes with zero readcounts in the input library were assigned a pseudocount of 0.999. A z-score for each peptide in experimental samples was then calculated based on the peptide sequence read count and the mean and standard deviation of read counts within its assigned bin (Mina et al., Science 366, 599-606, 2019). To increase stringency of analysis, z-score averages were obtained only for peptides with z-score 2 for either two peptide barcode replicates or two patient sample replicates; all other average z-scores were set to zero. Sequence alignments, z-score calculations, and data analysis were performed using ad hoc Python scripts and visualized using Prism 9 software. The logo plot in FIG. 2C was performed as previously described (Barber et al., Mol Cell 81, 3650-3658, 2021) using Logomaker (Tareen and Kinney, Bioinformatics 36, 2272-2274, 2019).

dCas9-Antibody Fusion Experiments

Plasmids encoding ATC-inducible dCas9 (pdCas9-bacteria #44249) and constitutively expressed gRNA (pgRNA-bacteria #44251) were obtained from Addgene (Qi et al., Cell 152, 1173-1183, 2013). pdCas9-bacteria was modified to contain a C-terminal fusion of a nanobody that binds a peptide from β-catenin (nanobody BC2-Nb; Addgene #186420) (Braun et al., Nucleic Acids Res 41, 7429-7437, 2016; Traenkle et al., Mol Cell Proteomics 14, 707-723, 2015) or an scFv that binds the spike protein from SARS-CoV-2 (ultrapotent B1-182.1; Addgene #186421) (Wang et al., Science 373, 2021), in addition to a 6His tag for purification. These plasmids were co-transformed with pgRNA-bacteria encoding gRNA spacers 5′-TCCATAGATTTCTCCGTGAG-3′ (SEQ ID NO: 35) and 5′-TGTTAGTTGCCCCATATCTT-3′ (SEQ ID NO: 36), respectively, into BL21 E. coli. Protein expression and purification was performed as above, using only ATC for induction. GST fused to the beta catenin peptide recognized by the nanobody was also expressed and purified in a similar manner in BL21 (plasmid Addgene #186422). Recombinant spike protein ectodomain with stabilizing mutations was purchased from Sino Biological (40589-V08H4).

Approximately 10 μg of recombinant GST-beta catenin peptide or 4 μg spike protein were added to wells of a 96-well MaxiSorp plate (ThermoFisher) at ambient temperature for 2 h, shaking at 40 rpm. Wells were washed 6 times with 100 μL PBST and then treated with 100 μL of 100 mg/mL BSA at 23° C. for 1 h, shaking at 40 rpm. Wells were again washed with 6 times with 100 μL PBST. Mixtures of approximately 2 μg each dCas9-nanobody and dCas9-scFv complexed with their respective gRNA barcodes were then added to each well in a 20 μL final volume (diluted using storage buffer) and incubated at 4° C. for 16 h. Wells were then washed with 12×100 μL PBST. 20 μL water was then added to each well, and the plate was heated to 100° C. in an oven for 10 min to elute gRNAs. 11 μL eluate was then used at the template for reverse transcription using SuperScript IV (Thermo) and the manufacturer's recommended protocols using primer 5′-GCACCGACTCGGTGCCACTTTTTC-3′ (SEQ ID NO: 37). gRNAs were then amplified using barcode-specific primers (i.e. one primer anneals within the gRNA spacer region: 5′-TCCATAGATTTCTCCGTGAG-3′ (SEQ ID NO: 38) or 5′-TGTTAGTTGCCCCATATCTTG-3′ (SEQ ID NO: 39), with common reverse primer 5′-GCACCGACTCGGTG-3′ (SEQ ID NO: 40) using Q5 (NEB) with 63° C. annealing temperature, 10 s extension, and 45-50 total amplification cycles. Amplicons were run on a 2% w/v agarose gel and visualized with UV light. Amplicon band intensities were measured using ImageJ.

Microarray-based experiments using dCas9-scFv fusion B1-182.1 were performed by adding approximately 1 μg of the purified dCas9-fusion (with gRNA spacer 5′-TCCATAGATTTCTCCGTGAG-3′ (SEQ ID NO: 41)) and a negative control dCas9-6His fusion (with gRNA spacer 5′-CCGTACCTAGATACACTCAA-3′ (SEQ ID NO: 42)) to a double stranded DNA microarray harboring triplicate complementary probe sequences. The microarray was incubated for 16 h at 37° C. The microarray was then blocked with 2% milk in PBST for 30 min at 23° C. Then, approximately 100 ng of purified recombinant SARS-CoV-2 spike protein (Sino Biological 40589-V08H4 was added to the microarray and incubated at 23° C. for 1 h. After washing twice with 40 μL PBST, 1:100 anti-SARS-CoV-2 spike CR3022 human IgG antibody (Cell Signaling 37475) was added to the microarray and incubated at 23° C. for 1 h. The microarray was then washed twice with PBST and then incubated with 1:40 Alexa 647-conjugated anti-human IgG Fc antibody (Biolegend 410714) at 23° C. for 1 h. The microarray was then visualized using a Genepix 4300A microarray scanner, and fluorescence intensities were extracted and analyzed as previously described (Barber et al., Mol Cell 81, 3650-3658, 2021). (Shrock et al., Science 370, eabd4250, 2020; Xu et al., Science 348, aaa0698, 2015) (Xu et al., Science 348, aaa0698, 2015) (Xu et al., Science 348, aaa0698, 2015) (Langmead et al., Genome Biol 10, R25, 2009) (Mina et al., Science 366, 599-606, 2019).

### OTHER EMBODIMENTS

All publications, patents, and patent applications mentioned in this specification are incorporated herein by reference to the same extent as if each independent publication or patent application was specifically and individually indicated to be incorporated by reference.

The invention includes the following numbered paragraphs.

### Methods of Making Libraries

1. A method for making a fusion protein library for use in a self-assembling protein microarray, the method comprising, for each member of the library, providing or making a Cas-containing fusion protein, wherein the Cas-containing fusion protein comprises:
(a) a catalytically inactive Cas-related protein;
(b) a protein of interest, wherein the protein of interest is linked to the Cas-related protein; and
(c) a single guide RNA (sgRNA), wherein the sgRNA comprises a unique nucleotide sequence complementary to a target sequence of a DNA probe.

2. A method for making a fusion protein for use in protein immobilization of a single protein on a non-microarray surface, the method comprising providing or making a Cas-containing fusion protein, wherein the Cas-containing fusion protein comprises:
(a) a catalytically inactive Cas-related protein;
(b) a protein of interest, wherein the protein of interest is linked to the Cas-related protein; and
(c) a sgRNA, wherein the sgRNA comprises a unique nucleotide sequence complementary to a target sequence of a DNA probe.

3. A method for making a fusion protein library for use in protein immobilization on a non-microarray surface, the method comprising, for each member of the library, providing or making a Cas-containing fusion protein, wherein the Cas-containing fusion protein comprises:
(a) a catalytically inactive Cas-related protein;
(b) a protein of interest, wherein the protein of interest is linked to the Cas-related protein; and
(c) a sgRNA, wherein the sgRNA comprises a unique nucleotide sequence complementary to a target sequence of a DNA probe.

4. The method of paragraph 1, further comprising causing the self-assembling protein microarray to self-assemble, the method comprising the steps of:
(i) making or providing a surface to which a plurality of DNA probes is attached, wherein each DNA probe comprises a target sequence; and
(ii) contacting the plurality of DNA probes with the fusion protein library under conditions that allow the specific hybridization of each sgRNA with its complementary target sequence, thus immobilizing each Cas-containing fusion protein on the surface.

5. The method of any one of paragraphs 1-4, wherein the catalytically inactive Cas-related protein is a catalytically inactive Cas9, Cas12a, or Cas14 protein.

6. The method of paragraph 5, wherein the catalytically inactive Cas9 protein is dCas9.

7. The method of any one of paragraphs 1-6, wherein the protein of interest is fused to the C terminus of the Cas-related protein.

8. The method of any one of paragraphs 1-6, wherein the protein of interest is fused to the N terminus of the Cas-related protein.

9. The method of any one of paragraphs 1-8, wherein the protein of interest is a viral protein or a fragment thereof.

10. The method of paragraph 9, wherein the viral protein is a SARS-CoV-2 protein or a fragment thereof.

11. The method of paragraph 9, wherein the viral protein is a human immunodeficiency virus (HIV) protein, an influenza A protein, a hepatitis C protein, a common coronaviruses like HKU1 protein, or an Ebola protein or a fragment thereof.

12. The method of any one of paragraphs 4-11, wherein each DNA probe comprises a 3′ universal annealing sequence; a target sequence, wherein the target sequence is complementary to an sgRNA spacer sequence; a protospacer adjacent motif (PAM) sequence; and a 5′ universal sequence.

13. The method of any one of paragraphs 4-11, wherein each DNA probe comprises the target sequence adjacent to the PAM sequence.

14. The method of paragraph 13, wherein each DNA probe is attached to a solid surface.

15. The method of any one of paragraphs 4-14, wherein each DNA probe is tethered to the support at its 3′ end.

16. The method of any one of paragraphs 4-14, wherein each DNA probe is tethered to the support at its 5′ end.

17. The method of any one of paragraphs 4-16, wherein each DNA probe is single-stranded.

18. The method of any one of paragraphs 4-16, wherein each DNA probe is partially or completely double-stranded.

19. The method of any one of paragraphs 4-18, wherein no two DNA probes share more than 50% sequence identity in the target sequence.

20. The method of any one of paragraphs 12-19, wherein the sgRNA spacer sequence has at least 50% sequence complementarity with the target sequence of any unique DNA probe.

21. The method of any one of paragraphs 12-19, wherein 6 or more bases in the DNA target sequence adjacent to the PAM motif are complementary to the bases on the 3′ end of the sgRNA spacer sequence.

22. The method of any one of paragraphs 1-21, wherein the sgRNA further comprises a 5′ constant region located 5′ to the sgRNA spacer sequence.

23. The method of any one of paragraphs 1-3, wherein making each Cas-containing fusion protein comprises
(i) making or providing a single plasmid comprising a nucleotide sequence encoding the Cas-containing fusion protein and a nucleotide sequence encoding the sgRNA; and
(ii) causing the fusion protein and the sgRNA to be expressed and to assemble into a fusion protein-sgRNA complex.

24. The method of any one of paragraphs 1-3, wherein making each Cas-containing fusion protein comprises
(i) making or providing a pair of plasmids, wherein a first plasmid of the pair comprises a nucleotide sequence encoding the Cas-containing fusion protein and a second plasmid of the pair comprises a nucleotide sequence encoding the sgRNA; and
(ii) causing the fusion protein and the sgRNA to be expressed and to assemble into a fusion protein-sgRNA complex.

25. The method of paragraph 23 or 24, wherein the plasmid or plasmids are comprised by a host cell.

26. The method of paragraph 25, wherein the host cell is a bacterial cell, a mammalian cell, or a yeast cell.

27. The method of paragraph 26, wherein the bacterial cell is an E. coli cell.

28. The method of paragraph 23 or 24, wherein the method is performed in an in vitro reaction.

29. The method of paragraph 28, wherein the in vitro reaction comprises an emulsion step, and wherein an emulsion droplet of the emulsion step comprises the fusion protein and the sgRNA.

30. The method of any one of paragraphs 1-29, wherein the fusion protein library comprises at least two unique Cas-containing fusion proteins.

31. The method of paragraph 30, wherein the fusion protein library comprises 100, 1,000, 10,000, 100,000, 125,000, 250,000, 500,000, 750,000, or 1,000,000 unique Cas-containing fusion proteins.

32. The method of any one of paragraphs 1-31, wherein the protein of interest is 8-40 amino acids in length.

33. The method of any one of paragraphs 1-31, wherein the protein of interest is greater than 40 amino acids in length.

### Method of Using Surfaces Including Microarrays or Non-Micro Arrays

34. The method of paragraph 4, further comprising contacting the protein microarray with a biological sample under conditions that would allow a specific reaction between a Cas-containing fusion protein of interest of the fusion protein library and a moiety in the biological sample.

35. The method of paragraph 2 or 3, wherein the non-microarray surface is a wire, a smart material, a hydrogel, or any other suitable solid material.

36. The method of paragraph 2 or 3, further comprising contacting the non-microarray surface with a biological sample under conditions that would allow a specific reaction between a Cas-containing fusion protein of interest of the fusion protein library and a moiety in the biological sample.

37. The method of paragraph 22, further comprising amplifying the sgRNA using a 5′ constant region located 5′ to the sgRNA spacer sequence using a sequencing-based method.

38. The method of paragraph 37, wherein the sequence-based method comprises a polymerase chain reaction (PCR), a real-time PCR, or nucleic acid sequencing.

39. The method of paragraph 34, further comprising identifying a reaction between a fusion protein of interest of the fusion protein library and a moiety in the biological sample by detecting a specific reaction.

40. The method of paragraph 34 or 39, wherein the reaction is an interaction.

41. The method of paragraph 34 or 39, wherein the protein of interest comprised by the Cas-containing fusion protein is pathogen-associated.

42. The method of paragraph 41, wherein the pathogen-associated protein is a SARS-CoV-2 protein or a fragment thereof.

43. The method of paragraph 34 or 39, wherein the protein of interest comprised by the Cas-containing fusion protein is a viral protein or a fragment thereof.

44. The method of paragraph 43, wherein the viral protein is a HIV protein, an influenza A protein, a hepatitis C protein, a common coronaviruses like HKU1 protein, or an Ebola protein or a fragment thereof.

45. The method of paragraph 41, wherein the pathogen-associated protein is a viral pathogen-associated protein.

46. The method of paragraph 45, wherein the viral pathogen-associated protein is a SARS-CoV-2 protein.

47. The method of paragraph 34 or 39, wherein the protein of interest comprised by the Cas-containing fusion protein corresponds to a protein or a fragment thereof in the proteome of an organism (for example, a bacterium, a virus, a fungus, an animal (for example, a human), a plant, or an invertebrate.

48. The method of paragraph 47, wherein the protein of interest is synthetic.

49. The method of paragraph 39, 41, or 47, wherein the protein of interest comprised by the Cas-containing fusion protein is an antibody or an antibody-like protein or peptide.

50. The method of any one of paragraphs 39, 41, or 47, wherein the moiety is an antibody or a disease biomarker.

51. The method of paragraph 50, wherein the antibody is an antiviral antibody.

52. The method of paragraph 51, wherein the antiviral antibody is an anti-SARS-CoV-2 antibody.

### Cas-Containing Fusion Protein

53. A Cas-containing fusion protein comprising
(a) a catalytically inactive Cas-related protein;
(b) a protein of interest, wherein the protein of interest is linked to the Cas-related protein; and
(c) a sgRNA, wherein the sgRNA comprises a unique nucleotide sequence complementary to a target sequence of a DNA probe.

54. The Cas-containing fusion protein of paragraph 53, wherein the catalytically inactive Cas-related protein is a catalytically inactive Cas9, Cas12a, or Cas14 protein.

55. The Cas-containing fusion protein of paragraph 54, wherein the catalytically inactive Cas9 protein is dCas9.

56. The Cas-containing fusion protein of any one of paragraphs 53-55, wherein the protein of interest is fused to the C terminus of the Cas-related protein.

57. The Cas-containing fusion protein of any one of paragraphs 53-55, wherein the protein of interest is fused to the N terminus of the Cas-related protein.

58. The Cas-containing fusion protein of any one of paragraphs 53-57, wherein each DNA probe comprises a 3′ universal annealing sequence; a target sequence, wherein the target sequence is complementary to an sgRNA spacer sequence; a PAM sequence; and a 5′ universal sequence.

59. The Cas-containing fusion protein of any one of paragraphs 53-58, wherein each DNA probe comprises the target sequence adjacent to the PAM sequence.

60. The Cas-containing fusion protein of paragraph 59, wherein the DNA probe is attached to a solid surface.

61. The Cas-containing fusion protein of any one of paragraphs 53-60 wherein the protein of interest is a viral protein or a fragment thereof.

62. The Cas-containing fusion protein of paragraph 61, wherein the viral protein is a SARS-CoV-2 protein or a fragment thereof.

63. The Cas-containing fusion protein of paragraph 62, wherein the viral protein is a HIV protein, an influenza A protein, a hepatitis C protein, a common coronaviruses like HKU1 protein, or an Ebola protein or a fragment thereof.

64. The Cas-containing fusion protein of any one of paragraphs 53-63, wherein each DNA probe is tethered to the support at its 3′ end.

65. The Cas-containing fusion protein of any one of paragraphs 53-63, wherein each DNA probe is tethered to the support at its 5′ end.

66. The Cas-containing fusion protein of any one of paragraphs 53-65, wherein each DNA probe is single-stranded.

67. The Cas-containing fusion protein of any one of paragraphs 53-65, wherein each DNA probe is partially or completely double-stranded.

68. The Cas-containing fusion protein of any one of paragraphs 53-67, wherein no two DNA probes share more than 50% sequence identity in the target sequence.

69. The Cas-containing fusion protein of any one of paragraphs 53-68, wherein the sgRNA spacer sequence has at least 50% sequence complementarity with the target sequence of any unique DNA probe.

70. The Cas-containing fusion protein of any one of paragraphs 53-68, wherein 6 or more bases in the DNA target sequence adjacent to the PAM motif are complementary to the bases on the 3′ end of the sgRNA spacer sequence.

71. The Cas-containing fusion protein of any one of paragraphs 53-70, wherein the sgRNA further comprises a 5′ constant region located 5′ to the sgRNA spacer sequence.

### Fusion Protein Library

72. A fusion protein library, the library comprising a plurality of Cas-containing fusion proteins, wherein each Cas-containing fusion protein comprises:
(a) a catalytically inactive Cas-related protein;
(b) a protein of interest, wherein the protein of interest is linked to the Cas-related protein; and
(c) a sgRNA, wherein the sgRNA comprises a unique nucleotide sequence complementary to a target sequence of a DNA probe.

### Plasmid Library

73. A plasmid library, the library comprising a plurality of plasmids encoding Cas-containing fusion proteins, wherein each plasmid encodes:
(a) a catalytically inactive Cas-related protein;
(b) a protein of interest, wherein the protein of interest is linked to the Cas-related protein; and
(c) a sgRNA, wherein the sgRNA comprises a unique nucleotide sequence complementary to a target sequence of a DNA probe;

74. The plasmid library of paragraph 73, wherein the sgRNA further comprises a 5′ constant region located 5′ to the sgRNA spacer sequence.

### Capture Complex

75. A capture complex, the complex comprising:
(i) a DNA probe, wherein the DNA probe comprises a target sequence and is attached to a surface; and
(ii) a Cas-containing fusion protein, wherein the Cas-containing fusion protein comprises:
(a) a catalytically inactive Cas-related protein;
(b) a protein of interest, wherein the protein of interest is linked to the Cas-related protein; and
(c) a sgRNA, wherein the sgRNA comprises a unique nucleotide sequence complementary to the target sequence of a DNA probe;
wherein the fusion protein is localized to the surface by base pairing interaction between the unique nucleotide sequence of the sgRNA and the target sequence of the DNA probe, thus forming the capture complex.

76. The capture complex of paragraph 75, wherein the sgRNA further comprises a 5′ constant region located 5′ to the sgRNA spacer sequence.

### Host Cell

77. A composition comprising a host cell comprising a pair of plasmids, wherein a first plasmid of the pair comprises a nucleotide sequence encoding a Cas-containing fusion protein and a second plasmid of the pair comprises a nucleotide sequence encoding a sgRNA.

78. The composition of paragraph 77, wherein the host cell is a bacterial cell, a mammalian cell, or a yeast cell.

79. The composition of paragraph 78, wherein the bacterial cell is an E. coli cell.

### Surfaces

80. A surface comprising
(a) a nucleic acid molecule; and
(b) a Cas-related protein comprising (i) an sgRNA and (ii) a protein of interest.

81. The surface of paragraph 80, wherein the nucleic molecule is DNA or RNA.

82. The surface of paragraph 80, wherein the Cas-related protein is a catalytically inactive Cas9, Cas12a, Cas13, or Cas14 protein.

83. The surface of paragraph 80, wherein the protein of interest is an epitope tag, a viral protein, a bacterial protein, a parasitic protein, or an animal protein.

84. The surface of paragraph 83, wherein the epitope tag is 6His-HA, 6His-myc, 6His-FLAG, or 6His.

85. The surface of paragraph 80, wherein the surface is a microarray or a non-microarray surface.

### Other Compositions

86. A composition comprising a Cas-related protein comprising (i) an sgRNA and (ii) a protein of interest.

87. The composition of paragraph 86, wherein the nucleic molecule is DNA or RNA.

88. The composition of paragraph 86, wherein the Cas-related protein is a catalytically inactive Cas9, Cas12a, Cas13, or Cas14 protein.

89. The composition of paragraph 86, wherein the protein of interest is an epitope tag, a viral protein, a bacterial protein, a parasitic protein, or an animal protein.

90. The composition of paragraph 89, wherein the epitope tag is 6His-HA, 6His-myc, 6His-FLAG, or 6His.

91. The composition of paragraph 86, wherein the composition comprises a nucleic acid molecule, wherein said nucleic acid molecule binds said Cas-related protein.

What is claimed is:

1. A method for making a fusion protein library, the method comprising, for each member of the library, providing or making a Cas-containing fusion protein, wherein the Cas-containing fusion protein comprises:
(a) a catalytically inactive Cas-related protein;
(b) a protein of interest, wherein the protein of interest is linked to the Cas-related protein; and
(c) a single guide RNA (sgRNA), wherein the sgRNA comprises a unique nucleotide sequence.

2. The method of claim 1, wherein the sgRNA is utilized for sgRNA sequencing.

3. The method of claim 1, wherein the sgRNA is complementary to a target sequence of a DNA probe.

4. A method for making a fusion protein for use in protein immobilization of a single protein on a non-microarray surface, the method comprising providing or making a Cas-containing fusion protein, wherein the Cas-containing fusion protein comprises:
(a) a catalytically inactive Cas-related protein;
(b) a protein of interest, wherein the protein of interest is linked to the Cas-related protein; and
(c) a sgRNA, wherein the sgRNA comprises a unique nucleotide sequence complementary to a target sequence of a DNA probe.

5. A method for making a fusion protein library for use in protein immobilization on a non-microarray surface, the method comprising, for each member of the library, providing or making a Cas-containing fusion protein, wherein the Cas-containing fusion protein comprises:
(a) a catalytically inactive Cas-related protein;
(b) a protein of interest, wherein the protein of interest is linked to the Cas-related protein; and
(c) a sgRNA, wherein the sgRNA comprises a unique nucleotide sequence complementary to a target sequence of a DNA probe.

6. The method of claim 1, further comprising causing a self-assembling protein microarray to self-assemble, the method comprising the steps of:
(i) making or providing a surface to which a plurality of DNA probes is attached, wherein each DNA probe comprises a target sequence; and
(ii) contacting the plurality of DNA probes with the fusion protein library under conditions that allow the specific hybridization of each sgRNA with its complementary target sequence, thus immobilizing each Cas-containing fusion protein on the surface.

7. The method of claim 6, wherein each DNA probe comprises a 3′ universal annealing sequence; a target sequence, wherein the target sequence is complementary to an sgRNA spacer sequence; a protospacer adjacent motif (PAM) sequence; and a 5′ universal sequence.

8. The method of claim 7, wherein each DNA probe comprises the target sequence adjacent to the PAM sequence.

9. The method of claim 8, wherein each DNA probe is attached to a solid surface.

10. The method of claim 1, wherein the sgRNA further comprises a 5′ constant region or a primer annealing region located 5′ to the sgRNA spacer sequence.

11. The method of any one of claims 1, 2, 3, 4 or 5, wherein making each Cas-containing fusion protein comprises:
(i) making or providing a single plasmid comprising a nucleotide sequence encoding the Cas-containing fusion protein and a nucleotide sequence encoding the sgRNA; and
(ii) causing the fusion protein and the sgRNA to be expressed and to assemble into a fusion protein-sgRNA complex.

12. The method of claim 11, wherein the method is performed in vitro or in vivo (such as utilizing a plasmid or plasmids which are comprised by a host cell).

13. The method of any one of claims 1, 2, 3, 4 or 5, wherein making each Cas-containing fusion protein comprises:
(i) making or providing a pair of plasmids, wherein a first plasmid of the pair comprises a nucleotide sequence encoding the Cas-containing fusion protein and a second plasmid of the pair comprises a nucleotide sequence encoding the sgRNA; and
(ii) causing the fusion protein and the sgRNA to be expressed and to assemble into a fusion protein-sgRNA complex.

14. The method of claim 13, wherein the method is performed in vitro.

15. The method of claim 13, wherein the plasmid or plasmids are comprised by a host cell.

16. The method of claim 15, wherein the host cell is a bacterial cell, a mammalian cell, or a yeast cell.

17. The method of claim 6, further comprising contacting the protein microarray with a sample (e.g., a biological sample) under conditions that would allow a specific reaction between a Cas-containing fusion protein of interest of the fusion protein library and a moiety in the sample.

18. The method of claim 17, wherein the protein of interest comprised by the Cas-containing fusion protein is pathogen-associated.

19. The method of claim 17, wherein the protein of interest comprised by the Cas-containing fusion protein corresponds to a protein or a fragment thereof in the proteome of an organism, for example, a bacterium, a virus, a fungus, an animal, a plant, or an invertebrate.

20. The method of claim 17, wherein the protein of interest comprised by the Cas-containing fusion protein is an antibody or an antibody-like protein or peptide.

21. The method of claim 17, wherein the moiety is an antibody or a disease biomarker.

22. The method of claim 10, further comprising amplifying the sgRNA using the 5′ constant region or a primer annealing region located 5′ to the sgRNA spacer sequence using a sequencing-based method.

23. The method of claim 1, 2, 3, 4 or 5, further comprising identifying a reaction between a fusion protein of interest of the fusion protein library and a moiety in the sample by detecting a specific reaction.

24. The method of claim 23, wherein the protein of interest comprised by the Cas-containing fusion protein is pathogen-associated.

25. The method of claim 23, wherein the protein of interest comprised by the Cas-containing fusion protein corresponds to a protein or a fragment thereof in the proteome of an organism, for example, a bacterium, a virus, a fungus, an animal, a plant, or an invertebrate.

26. The method of claim 23, wherein the protein of interest comprised by the Cas-containing fusion protein is an antibody or an antibody-like protein or peptide.

27. The method of claim 23, wherein the moiety is an antibody or a disease biomarker.

28. A Cas-containing fusion protein library, wherein each member of the library comprises:
(a) a catalytically inactive Cas-related protein;
(b) a protein of interest, wherein the protein of interest is linked to the Cas-related protein; and
(c) a sgRNA, wherein the sgRNA comprises a unique nucleotide sequence

29. The library of claim 28, wherein each sgRNA is complementary to a target sequence of a DNA probe.

30. The library of claim 29, wherein each Cas-containing fusion protein is in association with DNA probe on a surface.

31. The library of claim 28, wherein the sgRNA comprises a 5′ primer annealing region.

32. The library of claim 30, wherein the surface contains a plurality of DNA probes, wherein no two DNA probes share more than 50% sequence identity within the sgRNA-complementary target sequence.

33. The library of claim 28, 29, or 30, wherein the sgRNA spacer sequence has at least 50% sequence complementarity with the target sequence of any unique DNA probe.

34. The library of claim 28, wherein the sgRNA further comprises a 5′ constant region or a primer annealing region located 5′ to the sgRNA spacer sequence.

35. A plasmid library, the library comprising a plurality of plasmids encoding Cas-containing fusion proteins, wherein each plasmid encodes:
(a) a catalytically inactive Cas-related protein;
(b) a protein of interest, wherein the protein of interest is linked to the Cas-related protein; and
(c) a sgRNA, wherein the sgRNA comprises a unique nucleotide sequence complementary to a target sequence of a DNA probe.

36. A capture complex, the complex comprising:
(i) a DNA probe, wherein the DNA probe comprises a target sequence; and
(ii) a Cas-containing fusion protein complex, wherein the Cas-containing fusion protein complex comprises:
(a) a catalytically inactive Cas-related protein;
(b) a protein of interest, wherein the protein of interest is linked to the Cas-related protein; and
(c) a sgRNA, wherein the sgRNA comprises a unique nucleotide sequence.

37. The capture complex of claim 36, wherein DNA probe is attached to a surface.

38. The capture complex of claim 36, wherein the sgRNA comprises a unique nucleotide sequence complementary to the target DNA sequence of a DNA probe.

39. The capture complex of claim 36, wherein the fusion protein is localized to the surface by base pairing interaction between the unique nucleotide sequence of the sgRNA and the target sequence of the DNA probe.

40. A surface comprising:
(a) a nucleic acid molecule; and
(b) a Cas-related protein complex comprising (i) an sgRNA and (ii) a protein of interest, wherein the Cas-related protein is fused to the protein of interest which is bound to the sgRNA.

41. The surface of claim 40, wherein the surface is a microarray or a non-microarray surface.

42. The surface of claim 40, wherein the protein of interest is a synthetic antibody, a pathogen-derived protein, a mammalian protein, or a mutant protein variant thereof of a pathogen derived protein or a mammalian protein.
